(19) United States
(12) Patent Application Publication
(10) Pub. No.: US 2020/0222536 A1
(43) Pub. Date: Jul. 16, 2020

(54) BIOPHOTONIC COMPOSITIONS COMPRISING A FUNGAL-DERIVED CHROMOPHORE

(71) Applicants: Francesco BELLINI, Calgary (CA); ORPHADERM LIMITED, Dublin (IE)

(72) Inventors: Francesco BELLINI, Calgary (CA); Nikolaos LOUPIS, Athens (GR); Remigio PIERGALLINI, San Benedetto Del Tronto (IT); Andreas NIKOLIS, Ville Mont-Royal (CA); Giovanni SCAPAGNINI, Catania (IT)

(21) Appl. No.: 16/304,153

(22) PCT Filed: May 23, 2017
(86) PCT No.: PCT/CA2017/050621

§ 371 Date: Nov. 22, 2018

Related U.S. Application Data
(60) Provisional application No. 62/340,358, filed on May 23, 2016.

(51) Int. Cl. A61K 41/00; A61K 36/07; A61K 36/074; A61K 36/068; A61K 9/06; A61P 17/00; C09K 11/06; A61N 5/06

(52) U.S. Cl. CPC A61K 41/0057; A61K 36/07; A61K 36/074; A61K 36/068; A61N 5/0616; A61P 17/00; C09K 11/06; A61N 5/062; A61K 9/06

(57) ABSTRACT

The present disclosure provides biophotonic compositions comprising one or more fungal-derived chromophores and methods useful in phototherapy. In particular, the biophotonic compositions and the methods of the present disclosure are useful for the treatment of rare diseases that afflict skin or soft tissues. The present disclosure also provides a photoactivatable fabric composition in combination with the biophotonic compositions.

### CROSS-REFERENCE TO RELATED APPLICATION

This application claims priority to U.S. provisional patent application No. 62/340,358, filed May 23, 2016, the content of which is herein incorporated in its entirety by reference.

### BACKGROUND OF THE DISCLOSURE

Phototherapy is recognized as having a wide range of applications in both the medical and cosmetic fields. For example, phototherapy has been used to disinfect target sites as an antimicrobial treatment, to promote wound healing, and for skin rejuvenation.

One type of phototherapy comprises the topical application to a target tissue of compositions comprising chromophores. When activated by an incident light, the chromophores absorb and emit light such as through fluorescence with a therapeutic effect on its own and/or in combination with the incident light also irradiating the target tissue. Furthermore, the light activated chromophore may react with an oxygen source to generate oxygen radicals such as singlet oxygen which at low levels may also have a therapeutic effect on the target tissue.

In another type of phototherapy, known as photodynamic therapy, a photosensitizer is applied to a target tissue and after a determined period of time during which the photosensitizer is absorbed by cells, the target tissue is exposed to a light source. The activated photosensitizer generates oxygen radicals from within the cells leading to cell destruction. Photodynamic therapy finds uses in cancer and antimicrobial treatments where cell destruction is a required mechanism of action.

It is the object of the present disclosure to provide improved compositions and methods useful in phototherapy.

### SUMMARY OF THE DISCLOSURE

The present disclosure provides improved compositions for use in biophotonic therapy. In particular, a biophotonic composition of the present disclosure may include at least one fungal-derived chromophore and a carrier medium. In some embodiments, the at least one fungal-derived chromophore is derived from Grifola frondosa. In some embodiments, the at least one fungal-derived chromophore is derived from a Ganoderma species. In some embodiments, the at least one fungal-derived chromophore is derived from Laricifomes officinalis. In some embodiments, the at least one fungal-derived chromophore is derived from an Agaricus species. In some embodiments, the at least one fungal-derived chromophore is derived from a Tricholoma species. In some embodiments, the at least one fungal-derived chromophore is derived from a Cordyceps species. In some embodiments, the at least one fungal-derived chromophore is derived from a Lentinula species. In some embodiments, the biophotonic compositions of the disclosure comprise a combination of fungal-derived chromophores derived from Grifola frondosa, a Ganoderma species, Laricifomes officinalis, or, a Cordyceps species. In some embodiments, the biophotonic compositions of the disclosure comprise a combination of fungal-derived chromophores derived from Grifola frondosa, a Ganoderma species, Laricifomes officinalis, an Agaricus species, or a Tricholoma species.

In some embodiments of the foregoing or following, the composition further includes an oxidant or peroxide source. In certain embodiments of the foregoing or following, the oxidant or peroxide source is selected from hydrogen peroxide, carbamide peroxide, benzoyl peroxide, peroxy acid, alkali metal peroxide, alkali metal percarbonate, peroxyacetic acid, alkali metal perborate, methyl ethyl ketone peroxide, or combinations thereof. In some embodiments, the peroxide is carbamide peroxide. The peroxide or peroxide precursor may be present in the biophotonic composition in an amount of about 0.01% to about 50% by weight of the final composition.

In certain embodiments of the foregoing or following, the carrier medium comprises a hydrophilic polymer, a hygroscopic polymer, or a hydrated polymer, or combinations thereof. In some embodiments, the carrier medium is polyanionic in charge character. In some embodiments of the foregoing or following, the carrier medium comprises carboxylic functional groups. In some embodiments of the foregoing or following, the medium comprises a polymer having from 2 to 7 carbon atoms per functional group.

In certain embodiments of the foregoing or following, the carrier medium comprises a synthetic polymer selected from vinyl polymers, poly(ethylene oxide), acrylamide polymers, polyoxyethylene-polyoxypropylene copolymers, and derivatives or salts thereof and combinations thereof. In further embodiments, the carrier medium comprises one or more of a vinyl polymer selected from polyacrylic acid, polymethacrylic acid, polyvinyl pyrrolidone, and polyvinyl alcohol. The carrier medium may comprise a carboxy vinyl polymer or a carbomer obtained by polymerization of acrylic acid. The carboxy vinyl polymer or carbomer may be crosslinked. In some embodiments of the foregoing or following, the carrier medium comprises Carbopol® 940, Carbopol® 980, ETD 2020 NF, Carbopol® 1382 Polymer, 71G NF, 971P NF, 974P NF, 980 NF, 981 NF, 5984 EP, ETF 2020 NF, ultrez 10 NF, ultrez 20, ultrez 21, 1342 NF, 934 NF, 934P NF, 940 NF, or 941 NF, or combinations thereof. In some embodiments of the foregoing or following, the carrier medium comprises 2-Hydroxyethyl methacrylate (HEMA) either alone or in addition to another carrier. In some embodiments, the 2-Hydroxyethyl methacrylate (HEMA) is added to the carrier medium in the form of microspheres or in a further physically reduced form such as in a finely ground particulate form or in a pulverized, powder form. In some embodiments of the foregoing or following, the carrier medium comprises a polyacrylic acid polymer cross-linked with alkyl acrylate or allyl pentaerythritol. In some embodiments of the foregoing or following, the polymer is present in an amount of about 0.05% to about 5% by weight of the final composition, or about 0.1% to about 2.5%, or about 0.1% to about 2%, or about 0.5% to about 2.5%, or about 0.5% to about 2% by weight of the final composition. In some embodiments of the foregoing or following, the polymer is present in an amount of 0.05% to 5% by weight of the final composition, or 0.1% to 2.5%, or 0.1% to 2%, or 0.5% to 2.5%, or 0.5% to 2% by weight of the final composition.

In certain embodiments of the foregoing or following, the carrier medium comprises one or more protein-based polymers. In some embodiments, the protein-based polymer is gelatin or collagen, or both. In some embodiments, the carrier medium comprises gelatin. In some embodiments, gelatin is present in an amount of equal to or more than about 4% by weight of the final composition, such as 4% by weight of the final composition. In other embodiments, the carrier medium comprises collagen. In some embodiments, collagen is present in an amount equal to or more than about 5% by weight of the final composition, such as 5% by weight of the final composition.

In some embodiments, the carrier medium comprises sodium hyaluronate. In some embodiments, sodium hyaluronate is present in an amount of equal to or more than about 4% by weight of the final composition, such as 4% by weight of the final composition.

In certain embodiments of the foregoing or following, the carrier medium comprises one or more polysaccharides. In some embodiments, the polysaccharide is one or more of starch, chitosan, chitin, agar, alginates, xanthan, carrageenan, guar gum, gellan gum, pectin, or locust bean gum.

In some embodiments of the foregoing or following, the carrier medium comprises at least one glycol. In some embodiments, the glycol is one or more of ethylene glycol and propylene glycol.

In some embodiments of the foregoing or following, the carrier medium comprises a pharmaceutically acceptable medium.

The biophotonic compositions of the present disclosure comprise at least one chromophore that is derived from a fungal source. The at least one chromophore may be in the form of a molecular complex that conserves the photochemical properties of the at least one chromophore. Preferably, the chromophore or chromophores that are derived from a fungal source conserve their photochemical properties. In some embodiments, the at least one fungal-derived chromophore absorbs and/or emits light within the visible range. In some implementations of this embodiment, the at least one chromophore or molecular complex is extracted and/or isolated and/or purified from the at least one fungal source through methods and techniques known in the art. In some implementations, the at least one chromophore or molecular complex is in a form that is “purified”, “isolated” or “substantially pure”. The chromophore(s) or molecular complex(es) is said to be “purified”, “isolated” or “substantially pure” when it or they are separated from the components that naturally accompany them. Typically, a compound is substantially pure when it is at least 50%, 60%, 65%, 70%, 75%, 80%, 85%, 90%, 95%, 96%, 97%, 98%, or 99%, by weight, of the total composition in a sample.

In some embodiments, the biophotonic compositions of the present disclosure comprise a) a fungal extract comprising at least one fungal-derived chromophore; and b) a carrier medium comprising glycerin and propylene glycol. In some embodiments, the at least one fungal-derived chromophore is derived from a Ganoderma species (e.g., Ganoderma lucidum). In some embodiments, the composition comprises 4 parts fungal extract (part a) above) to 1 part carrier medium (part b) above) by volume. In some embodiments, the biophotonic composition further comprises at least a second chromophore (e.g., eosin or rose Bengal, or both).

In some aspects of the disclosure, there is provided biophotonic compositions prepared by the following steps: a) pulverizing at least one fungal source to provide a semi-fine, homogenous powder; b) adding five-fold excess of propylene glycol to said homogenous powder; c) stirring the resulting solution from step b) at low speed for at least 15 days; d) filtering the solution of step c) to obtain an extract comprising at least one fungal-derived chromophore; and e) combining the extract with a carrier medium.

In some embodiments of the foregoing or following, the biophotonic compositions as defined herein further comprise a chromophore-protecting agent such as, but not limited to, a buffer, a salt, and a solvent that preserves the photochemical activity or property of the chromophore(s).

In some embodiments of the foregoing or following, the at least one fungal-derived chromophore or molecular complex absorbs and/or emits light within the range of about 400 nm to about 750 nm. The at least one fungal-derived chromophore may absorb and/or emit light within the green, orange and yellow portions of the electromagnetic spectrum. In some embodiments of the foregoing or following, the at least one fungal-derived chromophore is derived from Grifola frondosa, a Ganoderma species, Laricifomes officinalis, an Agaricus species, a Tricholoma species, a Cordyceps species, or a Lentinula species. In some embodiments of the foregoing or following, the at least one fungal-derived chromophore is derived from Grifola frondosa. In some embodiments of the foregoing or following, the at least one fungal-derived chromophore is derived from a Ganoderma species. In some embodiments of the foregoing or following, the at least one fungal-derived chromophore is derived from Laricifomes officinalis. In some embodiments of the foregoing or following, the at least one fungal-derived chromophore is derived from an Agaricus species. In some embodiments of the foregoing or following, the at least one fungal-derived chromophore is derived from a Tricholoma species. In some embodiments of the foregoing or following, the at least one fungal-derived chromophore is derived from a Cordyceps species. In some embodiments of the foregoing or following, the at least one fungal-derived chromophore is derived from a Lentinula species. In some embodiments of the foregoing or following, the biophotonic compositions of the disclosure comprise a combination of fungal-derived chromophores derived from Grifola frondosa, a Ganoderma species, Laricifomes officinalis, or, a Cordyceps species. In some embodiments of the foregoing or following, the biophotonic compositions of the disclosure comprise a combination of fungal-derived chromophores derived from Grifola frondosa, a Ganoderma species, Laricifomes officinalis, an Agaricus species, or a Tricholoma species.

In some embodiments of the foregoing or following, the biophotonic composition further comprises at least a second chromophore or comprises a multiplicity of different chromophores. In some implementations of these embodiments, the at least second chromophore or any of the multiplicity of chromophores is derived from a fungus. In some embodiments, the at least second chromophore is a xanthene dye. In certain such embodiments, the xanthene dye is Eosin Y, Eosin B, Erythrosin B, Fluorescein, Rose Bengal, Phloxin B, or combinations thereof. In some embodiments, the xanthene dye is a combination of Eosin Y and Rose Bengal. In further implementations, the at least one fungal-derived chromophore has an emission spectrum that overlaps with an absorption spectrum of the at least second chromophore. In further embodiments, the at least one fungal-derived chromophore or molecular complex has an emission spectrum that overlaps at least 20% with an absorption spectrum of the at least second chromophore. The at least one fungal-derived chromophore or molecular complex may transfer energy to the at least second chromophore upon illumination with a light.

In certain embodiments of the foregoing or following, the biophotonic composition has a translucency of at least about 40%, about 45%, about 50%, about 55%, about 60%, about 65%, about 70%, about 75%, about 80%, about 85%, about 90%, about 91%, about 92%, about 93%, about 94%, about 95%, about 96%, about 97%, about 98%, about 99%, or about 100% in a visible range when measured without the chromophore(s) present. In some embodiments of the foregoing or following, the biophotonic composition has a translucency of at least 40%, 45%, 50%, 55%, 60%, 65%, 70%, 75%, 80%, 85%, 90%, 91%, 92%, 93%, 94%, 95%, 96%, 97%, 98%, 99%, or 100% in a visible range when measured without the chromophore(s) present.

In various embodiments of the foregoing or following, the present disclosure relates to an article of manufacture (e.g., a device) for use according to the methods disclosed herein. In some embodiments, the article of manufacture comprises a biophotonic composition of the present disclosure, and a photoactivatable fiber having a plurality of a photoactivatable strand (or a filament). The photoactivatable strand (or filament) comprises a first polymer and a second polymer, and at least one photoactivatable agent. In some embodiments, the first polymer forms a core along the length of the strand, and the second polymer forms a sheath surrounding the core along the length of the strand. In some embodiments, the at least one photoactivable agent absorbs and emits light between about 400 nm and about 800 nm. In some embodiments, the fiber is, but not limited to, synthetic fibers, natural fibers, and textile fibers. For example, synthetic fibers may be made from a polymer or a combination of different polymers, In some embodiments, the polymer is a thermoplastic polymer. In certain embodiments of the foregoing or following, the biophotonic composition is used for treatment of a rare disease that afflicts skin or soft tissues. In some embodiments, the rare disease that afflicts skin or soft tissues is selected from Hailey-Hailey syndrome, epidermolysis bullosa, CHILD syndrome, dermatomyositis, hidradenitis suppurativa, acquired ichthyosis, hereditary ichthyosis, lichen myxedematosus, scleromyxedema, pemphigus, a porphyria disorders, Ehlers-Danlos syndrome, cutis hyperelastica, eosinophilic fasciitis, osteogenesis imperfect, scleroderma, and Winchester syndrome. In some embodiments, the rare disease that afflicts skin or soft tissues is selected from Hailey-Hailey syndrome, epidermolysis bullosa, hidradenitis suppurativa, and scleroderma.

In another aspect, there is provided a method for biophotonic treatment of a rare disease that afflicts skin or soft tissues, wherein the method comprises applying a biophotonic composition to a target tissue (such as a skin tissue), wherein the biophotonic composition comprises a fungal-derived chromophore within a carrier medium (e.g., Gel X), and illuminating said biophotonic composition with light having a wavelength that is absorbed by the at least one fungal-derived chromophore. In some embodiments, the rare disease is selected from Hailey-Hailey syndrome, epidermolysis bullosa, CHILD syndrome, dermatomyositis, hidradenitis suppurativa, acquired ichthyosis, hereditary ichthyosis, lichen myxedematosus, scleromyxedema, pemphigus, a porphyria disorders, Ehlers-Danlos syndrome, cutis hyperelastica, eosinophilic fasciitis, osteogenesis imperfect, scleroderma, and Winchester syndrome. In some embodiments, the rare disease is selected from Hailey-Hailey syndrome, epidermolysis bullosa, hidradenitis suppurativa, and scleroderma.

In some embodiments of the foregoing or following, upon exposure to light, the biophotonic composition emits at least 25% to at least 99% more red, yellow and/or orange light than a composition lacking the at least one fungal-derived chromophore. In some embodiments, upon exposure to light, the biophotonic composition emits at least 1.25×, 1.5×, 1.75× or more red, yellow and/or orange light than a composition lacking the at least one fungal-derived chromophore. In other embodiments, upon exposure to light, the composition emits at least 5×, 10× or 20× more red, yellow and/or orange light than a composition lacking the at least one fungal-derived chromophore.

The light that may be useful for illumination of the biophotonic composition as defined herein is a continuous light. In some other implementations, the light that may be useful for illumination of the biophotonic composition as defined herein is a modulated light such as a pulsed light. In some implementations of this aspect, the light source that may be useful for illumination of the biophotonic composition as defined herein is a light-emitting diode (LED).

### BRIEF DESCRIPTION OF THE DRAWINGS

FIGS. 1A and 1B depict the absorbance (FIG. 1A) and fluorescence (FIG. 1B) values for four gel compositions, Blank Gel, Gel A, Gel B, and Gel C and blue light alone after irradiation of the compositions with a blue lamp. Blank gel comprises water and other components, but lacks a chromophore and urea peroxide. Gel A comprises water, a carbomer, urea peroxide and a combination of Maitake extract (aqueous, a Grifola species), Reishi extract (aqueous, a Ganoderma species), Agarikon extract (aqueous, a Laricifomes species), Cordyceps extract (aqueous), Rose Bengal and Eosin Y. Gel B comprises water, a carbomer, urea peroxide and a combination of Maitake extract (aqueous, a Grifola species), Reishi extract (aqueous, a Ganoderma species), Agarikon extract (aqueous, a Laricifomes species), Cordyceps extract (aqueous), and Rose Bengal. Gel C comprises water, a carbomer, urea peroxide and a combination of Maitake extract (aqueous, a Grifola species), Reishi extract (aqueous, a Ganoderma species), Agarikon extract (aqueous, a Laricifomes species), Agaricus extract (aqueous), Matsutake extract (aqueous, a Tricholoma species), and Eosin Y.

FIGS. 2A and 2B depict the fluorescence values for four gel compositions, Blank Gel, Gel A, Gel B, and Gel C and blue/green light alone after irradiation of the compositions with a blue/green lamp (84% lamp power, 115 mW). The gel compositions are the same as those for FIGS. 1A and 1B.

FIGS. 3A-3K provide results of urea peroxide stability analyses within various biophotonic compositions disclosed herein. As described in Example 7, urea stability was measured in the following conditions:1) urea peroxide and Pluronic (FIG. 3A); 2) urea peroxide, EDTA, and Pluronic (FIGS. 3B and 3C); 3) urea peroxide and Carrier Gel (FIGS. 3D and 3E); 4) 3%-12% urea peroxide and water (FIG. 3F); 5) urea peroxide and liquid carrier 20/15 or 15/15 (FIGS. 3G and 3H); 6) urea peroxide and liquid carrier 16.5% (FIG. 3I); 7) 15% urea peroxide in water (FIG. 3J); and 8) urea peroxide, Premix Pluronic, and EDTA (FIG. 3K).

FIGS. 4A-4D provide a GC-MS Plot (FIG. 4A) of Reishi extract and the corresponding data (FIGS. 4B-4D) from the study described in Example 8.

FIG. 5 summarizes the in vitro release test (Example 9) for various samples, including the homo chromophore mesh (“homo mesh”) and the sheath/core mesh of differing ratios with and without Gel, as indicated by fluorescence breakdown.

### DETAILED DESCRIPTION

### (1) Definitions

Before continuing to describe the present disclosure in further detail, it is to be understood that this disclosure is not limited to specific compositions or process steps, as such may vary. It must be noted that, as used in this specification and the appended claims, the singular form “a”, “an” and “the” include plural referents unless the context clearly dictates otherwise.

As used herein, the term “about” in the context of a given value or range refers to a value or range that is within 20%, preferably within 10%, and more preferably within 5% of the given value or range.

It is convenient to point out here that “and/or” where used herein is to be taken as specific disclosure of each of the two specified features or components with or without the other. For example “A and/or B” is to be taken as specific disclosure of each of (i) A, (ii) B and (iii) A and B, just as if each is set out individually herein.

“Biophotonic” means the generation, manipulation, detection and application of photons in a biologically relevant context. In other words, biophotonic compositions exert their physiological effects primarily due to the generation and manipulation of photons, for example, by absorbing photon to emit photons or to transfer energy, for example, by absorbing photons to emit photons or to transfer energy.

Terms “chromophore”, “photoactivating agent”, “photoactivatable agent”, and “photoactivator” are used herein interchangeably. A chromophore means a chemical compound, when contacted by light irradiation, is capable of absorbing the light. The chromophore readily undergoes photoexcitation and can transfer its energy to other molecules or emit it as light (e.g. fluorescence).

The term “actinic light” is intended to mean light energy emitted from a specific light source (e.g. lamp, LED, laser or sunlight) and capable of being absorbed by matter (e.g. the chromophore(s) or photoactivator(s)). The expression “actinic light” and the term “light” are used herein interchangeably. In some embodiments, the actinic light is visible light.

The term “oxidant” is intended to mean either a compound that readily transfers oxygen atoms and oxidizes other compounds, or a substance that gains electrons in a redox chemical reaction.

The term “reactive oxygen species” is intended to mean chemically-reactive molecules containing oxygen. Examples include oxygen ions and peroxides. They can be either inorganic or organic. Active oxygen species are highly reactive due to the presence of unpaired valence shell electrons. They are also referred to as “reactive oxygen”, “active oxygen”, or “active oxygen species”.

“Topical application”, “topical”, or “topical uses” means application to body surfaces, such as the skin, mucous membranes, vagina, oral cavity, internal surgical wound sites, and the like.

As used herein, the term “fiber” relates to a string or a thread used as a component of composite materials (e.g, fabric or mesh). Fibers may be used in the manufacture of other materials such as for example, but not limited to, fabrics (or mesh). Each fiber is composed of a plurality of (e.g., 19) “strands” or “filaments” of polymers. Each strand or filament that makes up the fiber, in turn, is configured in a sheath/core configuration. For example, a first polymer forms a core along the length of the strand or filament, and the second polymer forms a sheath surrounding the core polymer along the length of the strand or filament. To illustrate, in such a configuration, the cross-section of the strand, looking straight down along the long axis of the strand, would appear to be two concentric circles.

In some embodiments, a photoactivatable agent is associated with the first polymer (the core polymer of a strand). As used herein, “associated” refers to a photoactivatable agent being incorporated into the first polymer, by a method known in the art, e.g., compounding.

As used herein, “an article of manufacture” refers to the gel-mesh BioPhotonic System (BPS) disclosed herein. In some embodiments, the gel-mesh BPS System comprises Gel X as described herein and the mesh described herein.

In some instances, the polymer is acrylic, acrylonitrile butadiene styrene (ABS), polybenzimidazole (PBI), polycarbonate, polyether sulfone (PES), polyetherether ketone (PEEK), polyetherimide (PEI), polyethylene (PE), polyphenylene oxide (PPO), polyphenylene sulfide (PPS), polypropylene (PP), polystyrene, polyvinyl chloride (PVC), teflon, polybutylene, polyethylene terephthalate (PET), polybutylene terephthalate (PBT), nylon, polylactic acid (PLA), polymethyl methacrylate polyester, polyurethane, rayons, poly(methyl methacrylate) (PMMA), or from any mixture thereof.

In some other instances, the fibers may be made from glycolic acid, copolymer lactide/glycolide, polyester polymer, copolymer polyglycolic acid/trimethylene carbonate, natural protein fiber, cellulose fiber, polyamide polymer, polymer of polypropylene, polymer of polyethylene, nylon, polymer of polylactic acid, polymer of polybutylene terephthalate, polyester, copolymer polyglycol, polybutylene, polymer of poly methyl methacrylate, or from any mixture thereof.

In some implementations, the diameter of the photoactivatable fiber define herein (taken individually, monofilament) varies between about 15 microns and about 500 microns, between about 25 microns and about 500 microns, between about 50 microns and 400 microns, between about 50 microns and about 300 microns, preferably between about 50 microns and about 250 microns, preferably between about 75 microns and about 300 microns, and most preferably between about 75 microns and about 250 microns. In some specific implementations, the diameter of the photoactivatable fibers defined herein is about 15 microns, about 20 microns, about 25 microns, about 50 microns, about 75 microns, about 100 microns, about 1:25 microns, about 150 microns, about 175 microns, about 200 microns, about 225 microns, about 250 microns, about 250 microns, about 275 microns, about 300 microns, about 325 microns, about 350 microns, about 375 microns, about 400 microns, about 425 microns, about 450 microns, about 475 microns, about 500 microns. In some instances, the diameter of the photoactivatable fibers defined herein (taken individually) is about 31 microns.

In some implementations, the photoactivatable fibers defined herein show a medium to high resistance to mechanical pulling and stretching forces. In some implementations, the photoactivatable fibers defined here are resilient and have the ability to stretch and to reform to their original size and shape.

In some implementations, the photoactivatable fibers have a linear mass density of between about 400 and about 480 Deniers, between about 410 and about 470 Deniers, between about 420 and about 460 Deniers, between about 420 and about 450 Deniers, or about 428 Deniers. As used herein, the term “Denier” refers to a unit of measure for the linear mass density of fibers, is defined as the mass in grains per 9000 meters.

In some implementations, the fibers defined herein maintain their length and degree of flexibility and windability. In other implementation the stretch fibers may be lubricated to wind and unwind without damage being inflicted on the fibers due to the winding and the unwinding process. In some instance, the fibers have a tensile strength that allows the fibers to be stretched so as to reach a minimum diameter at least half, one third, one fourth, one fifth, one sixth, one seventh, one eight, one ninth, or one tenth of the original diameter.

Features and advantages of the subject matter hereof will become more apparent in light of the following detailed description of selected embodiments. As will be realized, the subject matter disclosed and claimed is capable of modifications in various respects, all without departing from the scope of the claims. Accordingly, the drawings and the description are to be regarded as illustrative in nature, and not as restrictive and the full scope of the subject matter is set forth in the claims.

### (2) Biophotonic Compositions Comprising Fungal-Derived Chromophores

The present disclosure provides, in a broad sense, biophotonic compositions which can be activated by light (e.g., photons) of specific wavelengths. A biophotonic composition according to various embodiments of the present disclosure contains at least one fungal-derived chromophore, or a molecular complex comprising the at least one fungal-derived chromophore, within a carrier medium. Activation of the chromophore(s) in the biophotonic composition may lead to the generation of oxygen radicals such as singlet oxygen, and in the case where the chromophore(s) is a fluorophore, may also lead to the generation of light of a different wavelength, each one of which individually or together may have a therapeutic effect.

When a chromophore absorbs a photon of a certain wavelength, it becomes excited. This is an unstable condition and the molecule tries to return to the ground state, giving away the excess energy. For some chromophores, it is favorable to emit the excess energy as light when returning to the ground state. This process is called fluorescence. The peak wavelength of the emitted fluorescence is shifted towards longer wavelengths compared to the absorption wavelengths due to loss of energy in the conversion process. This is called the Stokes' shift. In the proper environment (e.g., in a biophotonic composition) much of this energy is transferred to the other components of the biophotonic composition or to the treatment site directly.

Without being bound to theory, it is thought that fluorescent light emitted by photoactivated chromophores may have therapeutic properties due to its femto-, pico-, or nano-second emission properties which may be recognized by biological cells and tissues, leading to favorable biomodulation. Furthermore, the emitted fluorescent light has a longer wavelength and hence a deeper penetration into the tissue than the activating light. Irradiating tissue with such a broad range of wavelength, including in some embodiments, the activating light which passes through the composition, may have different and complementary effects on the cells and tissues. In other words, chromophores are used in the biophotonic compositions of the present disclosure for therapeutic effect on tissues. This is a distinct application of these photoactive agents and differs from the use of chromophores as simple stains or as catalysts for photo-polymerization.

The biophotonic compositions of the present disclosure may be described based on the components making up the composition. Additionally or alternatively, the compositions of the present disclosure have functional and structural properties and these properties may also be used to define and describe the compositions. Individual components of the biophotonic compositions of the present disclosure, including chromophores, oxidants (peroxides and peroxide precursors), carrier mediums and other optional ingredients, are detailed below.

Biophotonic Composition Gel X

In some embodiments, the biophotonic compositions of the present disclosure comprise a) a fungal extract comprising at least one fungal-derived chromophore; and b) a carrier medium comprising glycerin and propylene glycol. In some embodiments, the at least one fungal-derived chromophore is derived from a Ganoderma species (e.g., Ganoderma lucidum).

In some embodiments, the biophotonic composition comprises 4 parts fungal extract (part a) above) to 1 part carrier medium (part b) above) by volume. In some embodiments, the biophotonic composition comprises 4 parts fungal extract (part a) above) to 1 part carrier medium (part b) above) by volume. In some embodiments, the biophotonic composition comprises 5 parts fungal extract (part a) above) to 1 part carrier medium (part b) above) by volume. In some embodiments, the biophotonic composition comprises 3 parts fungal extract (part a) above) to 1 part carrier medium (part b) above) by volume. In some embodiments, the biophotonic composition comprises 2 parts fungal extract (part a) above) to 1 part carrier medium (part b) above) by volume. In some embodiments, the biophotonic composition comprises 1 part fungal extract (part a) above) to 1 part carrier medium (part b) above) by volume.

In some embodiments, the glycerin is present in the carrier medium in a range of about 5% to about 25% by w/w. In some embodiments, the glycerin is present in the carrier medium in a range of about 5% to about 15% by w/w. In some embodiments, the glycerin is present in the carrier medium in a range of about 5% to about 10% by w/w. In some embodiments, the glycerin is present in the carrier medium at about 11% by w/w.

In some embodiments, the propylene glylcol is present in the carrier medium in a range of about 40% to about 60% by w/w. In some embodiments, the propylene glylcol is present in the carrier medium in a range of about 30% to about 60% by w/w. In some embodiments, the propylene glylcol is present in the carrier medium in a range of about 30% to about 50% by w/w. In some embodiments, the propylene glylcol is present in the carrier medium in a range of about 40% to about 50% by w/w. In some embodiments, the propylene glylcol is present in the carrier medium at about 56% by w/w.

In some embodiments, the biophotonic composition further comprises at least a second chromophore, such as, for example, xanthene dye. In some embodiments, the xanthene dye is the xanthene dye is one or more of Eosin Y, Eosin B, Rose Bengal, or a combination thereof.

In some embodiments, the carrier medium further comprises an oxidant, such as, for example, urea peroxide. In some embodiments, the urea peroxide is present in the carrier medium in a range of about 10% to about 20% by w/w. In some embodiments, the urea peroxide is present in the carrier medium in a range of about 5% to about 15% by w/w. In some embodiments, the urea peroxide is present in the carrier medium in a range of about 15% to about 25% by w/w. In some embodiments, the urea peroxide is present in the carrier medium at about 16% by w/w.

In some embodiments, the biophotonic composition further comprises a thickening agent. In some embodiments, the thickening agent is an ethylene oxide (EO)-propylene oxide (PO) block copolymers (such as polymers sold under the trade mark Pluronic available from BASF Corporation).

In some embodiments, the biophotonic composition comprises the components, in the amounts as disclosed in Example 2 (the biphotonic composition referred to as Gel X).

In some embodiments, provided herein is a method of treating a rare disease that afflicts skin or soft tissues comprising administering a biophotonic composition (e.g., Gel X) of the present invention. In some embodiments, the rare disease is selected from Hailey-Hailey syndrome, epidermolysis bullosa, CHILD syndrome, dermatomyositis, hidradenitis suppurativa, acquired ichthyosis, hereditary ichthyosis, lichen myxedematosus, scleromyxedema, pemphigus, a porphyria disorders, Ehlers-Danlos syndrome, cutis hyperelastica, eosinophilic fasciitis, osteogenesis imperfect, scleroderma, and Winchester syndrome. In some embodiments, the rare disease is Hailey-Hailey syndrome. In some embodiments, the rare disease is epidermolysis bullosa. In some embodiments, the rare disease is hidradenitis suppurativa. In some embodiments, the rare disease is scleroderma.

(a) Chromophores

The biophotonic compositions, methods and uses of the present disclosure comprise at least one fungal-derived chromophore. In some embodiments, the at least one fungal-derived chromophore absorbs at a wavelength in the range of the visible spectrum, such as at a wavelength of about 380 nm-800 nm, about 380 nm-700 nm, about 400 nm-800 nm, or about 380 nm-600 nm. In other embodiments, the at least one fungal-derived chromophore absorbs at a wavelength of about 200 nm-800 nm, about 200 nm-700 nm, about 200 nm-600 nm or about 200 nm-500 nm. In some embodiments, the at least one fungal-derived chromophore absorbs at a wavelength of about 200 nm-600 nm. In some embodiments, the at least one fungal-derived chromophore absorbs light at a wavelength of about 200 nm-300 nm, about 250 nm-350 nm, about 300 nm-400 nm, about 350 nm-450 nm, about 400 nm-500 nm, about 450 nm-650 nm, about 600 nm-700 nm, about 650 nm-750 nm or about 700 nm-800 nm. In some embodiments, the at least one fungal-derived chromophore absorbs at a wavelength of 380 nm-800 nm, 380 nm-700 nm, 400 nm-800 nm, or 380 nm-600 nm. In other embodiments, the at least one fungal-derived chromophore absorbs at a wavelength of 200 nm-800 nm, 200 nm-700 nm, 200 nm-600 nm or 200 nm-500 nm. In some embodiments, the at least one fungal-derived chromophore absorbs at a wavelength of 200 nm-600 nm. In some embodiments, the at least one fungal-derived chromophore absorbs light at a wavelength of 200 nm-300 nm, 250 nm-350 nm, 300 nm-400 nm, 350 nm-450 nm, 400 nm-500 nm, 450 nm-650 nm, 600 nm-700 nm, 650 nm-750 nm or 700 nm-800 nm.

It will be appreciated to those skilled in the art that optical properties of a particular chromophore may vary depending on the chromophore's surrounding medium. Therefore, as used herein, a particular chromophore's absorption and/or emission wavelength (or spectrum) corresponds to the wavelengths (or spectrum) measured in a biophotonic composition of the present disclosure.

In some embodiments, the at least one fungal-derived chromophore is obtained from a fungal extract, for example, but not limited to, extracts of Chytridiomycetes, Blastocladiomycetes, Basidiobolomycetes, Deuteromycetes, Entomophthoromycetes, Kickxellomycetes, Mucoromycetes, Glomeromycetes, Entorrhizomycetes, Basidiomycetes, mushrooms, and yeast. In some embodiments, the at least one fungal-derived chromophore is derived from sources including, but not limited to, a Grifola species (e.g., Grifola frondosa (Maitake mushroom or Hen of the Woods)); a Ganoderma species (e.g., Ganoderma alba, Ganoderma annularis, Ganoderma atrum, Ganoderma aurea, Ganoderma amboinense, Ganoderma applanatum, Ganoderma brownie, Ganoderma curtisii, Ganoderma lobatum, Ganoderma lucidum, Ganoderma meredithiae, Ganoderma multipileum, Ganoderma nigrolucidum, Ganoderma orbiforme, Ganoderma oregonense, Ganoderma purpurea, Ganoderma philippii, Ganoderma pseudoferreum, Ganoderma rubra, Ganoderma sinense, Ganoderma tornatum, Ganoderma tsugae, Ganoderma viridis, Ganoderma zonatum, Ganoderma boninense, Ganoderma miniatocinctum, and lingzhi or reishi mushroom)); a Laricifomes species (e.g., Laricifomes officinalis (agarikon)); an Agaricus species (e.g., Agaricus bisporus (button mushroom), Agaricus campestris (field mushroom), Agaricus abruptibulbus, Agaricus agrinferus, Agaricus albolutescens, Agaricus aestivalis, Agaricus agrinferus, Agaricus annae, Agaricus alabamensis, Agaricus albertii, Agaricus altipes, Agaricus amicosus, Agaricus arcticus, Agaricus argentous, Agaricus argentines, Agaricus aridicola, Agaricus aristocratus, Agaricus arorae, Agaricus arvensis, Agaricus augustus, Agaricus aurantioviolaceus, Agaricus benesii, Agaricus bernardii, Agaricus biannulatus, Agaricus bisporiticus, Agaricus bitorquis, Agaricus blazei, Agaricus bohusianus, Agaricus bohusii, Agaricus bresadolanus, Agaricus caballeroi, Agaricus californicus, Agaricus campbellensis, Agaricus cellaris, Agaricus chartaceus, Agaricus chionodermus, Agaricus chlamydopus, Agaricus colpeteii, Agaricus comtuliformis, Agaricus comtulus, Agaricus cretacellus, Agaricus cretaceus, Agaricus crocodilinus, Agaricus cupreobrunneus, Agaricus cupressophilus, Agaricus depauperatus, Agaricus deserticola, Agaricus devoniensis, Agaricus diminutivus, Agaricus dulcidulus, Agaricus eburneocanus, Agaricus endoxanthus, Agaricus erthyrosarx, Agaricus essettei, Agaricus excellens, Agaricus fissuratus, Agaricus freirei, Agaricus fuscofibrillosus, Agaricus fuscovelatus, Agaricus geesterani syn. Allopsalliota geesterani, Agaricus floridanus, Agaricus fuscopunctatus, Agaricus haemorrhoidarius, Agaricus halophilus, Agaricus hondensis, Agaricus hortensis, Agaricus huijsmanii, Agaricus inilleasper, Agaricus impudicus, Agaricus inapertus, Agaricus koelerionensis, Agaricus lacrymabunda, Agaricus lamelliperditus, Agaricus langei, Agaricus lanipes, Agaricus laskibarii, Agaricus leucotrichus, Agaricus lilaceps, Agaricus litoralis, Agaricus ludovici, Agaricus luteomaculatus, Agaricus maclovianus, Agaricus macrocarpus, Agaricus macrolepis, Agaricus macrosporus, Agaricus magni, Agaricus maleolens, Agaricus medio-fuscus, Agaricus meleagris, Agaricus menieri, Agaricus micromegathus, Agaricus microvolvatulus, Agaricus minimus, Agaricus moelleri, Agaricus murinocephalus, Agaricus nebularum, Agaricus niveolutescens, Agaricus nivescens, Agaricus osecanus, Agaricus pachydermus, Agaricus pampeanus, Agaricus parvitigrinus, Agaricus pattersoniae, Agaricus perobscurus, Agaricus perrarus, Agaricus phaeolepidotus, Agaricus pilatianus, Agaricus pilosporus, Agaricus placomyces, Agaricus pocillator, Agaricus porphyrizon, Agaricus porphyrocephalus, Agaricus praerimosus, Agaricus pratensis, Agaricus pseudopratensis, Agaricus purpurellus, Agaricus radicatus, Agaricus romagnesii, Agaricus rosalamellatus, Agaricus rotalis, Agaricus rubellus, Agaricus rufotegulis, Agaricus rusiophyllus, Agaricus santacatalinensis, Agaricus semotus, Agaricus silvaticus, Agaricus silvicola, Agaricus smithii, Agaricus solidipes, Agaricus spissicaulis, Agaricus stigmaticus, Agaricus stramineus, Agaricus subantarcticus, Agaricus subfloccosus, Agaricus subperonatus, Agaricus subrufescens, Agaricus subrutilescens, Agaricus subsaharianus, Agaricus subsubensis, Agaricus taeniatus, Agaricus tlaxcalensis, Agaricus urinascens, Agaricus vaporarius, Agaricus variegans, Agaricus valdiviae, Agaricus xanthodermulus, Agaricus xanthodermus, and Agaricus xantholepis); a Tricholoma species (e.g., Tricholoma matsutake (Matsutake mushroom), Tricholoma acerbum, Tricholoma aestuans, Tricholoma albobrunneum, Tricholoma album, Tricholoma argyraceum, Tricholoma atrosquamosum, Tricholoma auratum, Tricholoma bakamatsutake, Tricholoma columbetta, Tricholoma equestre (previously T. flavovirens), Tricholoma flavum, Tricholoma huronense, Tricholoma imbricatum, Tricholoma inamoenum, Tricholoma magnivelare, Tricholoma mutabile, Tricholoma myomyces, Tricholoma nigrum, Tricholoma orirubens, Tricholoma pardinum, Tricholoma pessundatum, Tricholoma populinum, Tricholoma portentosum, Tricholoma resplendens, Tricholoma saponaceum, Tricholoma scalpturatum, Tricholoma sejunctum, Tricholoma squarrulosum, Tricholoma sulphureum, Tricholoma terreum (=T. myomyces), Tricholoma tigrinum, Tricholoma ustale, Tricholoma ustaloides, Tricholoma vaccinum, Tricholoma venenatum, Tricholoma virgatum, and Tricholoma zangii); a Cordyceps species (e.g., Cordyceps militaris); and a Lentinula species (e.g., Lentinula aciculospora, Lentinula boryana, Lentinula edodes (shiitake mushroom), Lentinula guarapiensis, Lentinula lateritia, Lentinula novae-zelandiae, Lentinula raphanica, and Lentinula reticeps. In some embodiments, the at least one fungal-derived chromophore is derived from Grifola frondosa, a Ganoderma species, Laricifomes officinalis, an Agaricus species, a Tricholoma species, a Cordyceps species, or a Lentinula species. In some embodiments, the biophotonic compositions of the disclosure comprise a combination of fungal-derived chromophores derived from Grifola frondosa, a Ganoderma species, Laricifomes officinalis, an Agaricus species, a Tricholoma species, a Cordyceps species, or a Lentinula species. In some embodiments, the biophotonic compositions of the disclosure comprise a combination of fungal-derived chromophores derived from Grifola frondosa, a Ganoderma species, Laricifomes officinalis, or, a Cordyceps species. In some embodiments, the biophotonic compositions of the disclosure comprise a combination of fungal-derived chromophores derived from Grifola frondosa, a Ganoderma species, Laricifomes officinalis, an Agaricus species, or a Tricholoma species.

Examples of fungal-derived chromophores include, but are not limited to, phloroglucinols, adhyperforin, terpenoids, polyphenols, stilbenoids, flavonoids, catechins, alkaloids, tannins, antraquinones, phytosterols, carotenoids, ergothioneine, isothiocyanates, quinones, pulvinic acids, grevillins, diarylcyclopentenones, pulvinones, benzotropolones, betacyanins, betaxanthins, phenoxazines, benzoquinones, terpenoid quinones, ketides, azaquinones, muscaurins, xanthones, scaurins, and derivatives thereof.

In some embodiments, the present disclosure provides biophotonic compositions prepared by the following steps: a) pulverizing at least one fungal source to provide a semi-fine, homogenous powder; b) adding five-fold excess of propylene glycol to said homogenous powder; c) stirring the resulting solution from step b) at low speed for at least 15 days; d) filtering the solution of step c) to obtain an extract comprising at least one fungal-derived chromophore; and e) combining the extract with a carrier medium.

In some embodiments of the disclosure, the at least one fungal-derived chromophore is extracted at least one fungal source. For example, the at least one fungal-derived chromophore can be extracted from a pulverized mushroom or from a cell pellet of yeast or a microorganism using an organic solvent such as acetone, benzene, chloroform, ethyl acetate, ethanol, methanol, petroleum ether, propylene glycol, hexane and DMSO. The at least one fungal-derived chromophore can then be purified by techniques such as column chromatography (reverse phase or silica gel), liquid chromatography, HPLC, thin layer chromatography (TLC), and gel permeation chromatography. The chromophore containing compositions resulting from the extraction or from the purification can be characterized using techniques such as UV-vis, FTIR, ESI-MS, and NMR.

The biophotonic compositions, methods, and uses disclosed herein may include at least one additional chromophore or a multiplicity of different chromophores. Combining chromophores may increase photo-absorption by the combined dye molecules and enhance absorption and photo-biomodulation selectivity. When such multi-chromophore compositions are illuminated with light, energy transfer can occur between the chromophores. This process, known as resonance energy transfer, is a widely prevalent photophysical process through which an excited ‘donor’ chromophore (also referred to herein as first chromophore) transfers its excitation energy to an ‘acceptor’ chromophore (also referred to herein as second chromophore). The efficiency and directedness of resonance energy transfer depends on the spectral features of donor and acceptor chromophores. In particular, the flow of energy between chromophores is dependent on a spectral overlap reflecting the relative positioning and shapes of the absorption and emission spectra. More specifically, for energy transfer to occur, the emission spectrum of the donor chromophore must overlap with the absorption spectrum of the acceptor chromophore.

Energy transfer manifests itself through decrease or quenching of the donor emission and a reduction of excited state lifetime accompanied also by an increase in acceptor emission intensity. To enhance the energy transfer efficiency, the donor chromophore should have good abilities to absorb photons and emit photons. Furthermore, the more overlap there is between the donor chromophore's emission spectra and the acceptor chromophore's absorption spectra, the better a donor chromophore can transfer energy to the acceptor chromophore.

In some embodiments, the biophotonic compositions, methods, and uses of the present disclosure further comprises at least a second chromophore. The at least second chromophore may be synthetic or fungal-derived. In certain embodiments, the at least one fungal-derived chromophore (i.e., the first chromophore) is the donor chromophore and the at least second chromophore is the acceptor chromophore. In other embodiments, the at least second chromophore is the donor chromophore and the at least one fungal-derived chromophore (i.e., the first chromophore) is the acceptor chromophore.

In some embodiments, the at least one fungal-derived chromophore (i.e., the first chromophore) has an emission spectrum that overlaps at least about 80%, about 75%, about 70%, about 65%, about 60%, about 55%, about 50%, about 45%, about 40%, about 35%, about 30%, about 25%, about 20%, about 15% or about 10% with an absorption spectrum of the at least second chromophore. In some embodiments, the least one fungal-derived chromophore (i.e., the first chromophore) has an emission spectrum that overlaps at least about 20% with an absorption spectrum of the at least second chromophore. In some embodiments, t the least one fungal-derived chromophore (i.e., the first chromophore) has an emission spectrum that overlaps at least between about 1%-10%, between about 5%-15%, between about 10%-20%, between about 15%-25%, between about 20%-30%, between about 25%-35%, between about 30%-40%, between about 35%-45%, between about 50%-60%, between about 55%-65% or between about 60%-70% with an absorption spectrum of the at least second chromophore.

In other embodiments, the at least second chromophore has an emission spectrum that overlaps at least about 80%, about 75%, about 70%, about 65%, about 60%, about 55%, about 50%, about 45%, about 40%, about 35%, about 30%, about 25%, about 20%, about 15% or about 10% with an absorption spectrum of the at least one fungal-derived chromophore (i.e., the first chromophore). In some embodiments, the at least second chromophore has an emission spectrum that overlaps at least about 20% with an absorption spectrum of the at least one fungal-derived chromophore (i.e., the first chromophore). In some embodiments, the at least second chromophore has an emission spectrum that overlaps at least between about 1%-10%, between about 5%-15%, between about 10%-20%, between about 15%-25%, between about 20%-30%, between about 25%-35%, between about 30%-40%, between about 35%-45%, between about 50%-60%, between about 55%-65% or between about 60%-70% with an absorption spectrum of the at least one fungal-derived chromophore (i.e., the first chromophore).

% spectral overlap, as used herein, means the % overlap of a donor chromophore's emission wavelength range with an acceptor chromophore's absorption wavelength range, measured at spectral full width quarter maximum (FWQM). For example, if the spectral FWQM of the acceptor chromophore's absorption spectrum is about 60 nm and the overlap of the donor chromophore's spectrum with the absorption spectrum of the acceptor chromophore is about 30 nm, then the % overlap can be calculated as 30 nm/60 nm×100=50%.

The at least one fungal-derived chromophore (i.e., the first chromophore) can be present in an amount of about 0.0001%-40% by weight of the biophotonic composition, such as 0.0001%-40% by weight of the biophotonic composition. When present, the at least second chromophore can be present in an amount of about 0.0001%-40% by weight of the biophotonic composition, such as 0.0001%-40% by weight of the biophotonic composition. When present, the third chromophore can be present in an amount of about 0.0001%-40% by weight of the biophotonic composition, such as 0.0001%-40% by weight of the biophotonic composition. In certain embodiments, the first chromophore is present in an amount of about 0.0001%-2%, about 0.001%-3%, about 0.001%-0.01%, about 0.005%-0.1%, about 0.1%-0.5%, about 0.5%-2%, about 1%-5%, about 2.5%-7.5%, about 5%-10%, about 7.5%-12.5%, about 10%-15%, about 12.5%-17.5%, about 15%-20%, about 17.5%-22.5%, about 20%-25%, about 22.5%-27.5%, about 25%-30%, about 27.5%-32.5%, about 30%-35%, about 32.5%-37.5%, or about 35%-40% by weight of the biophotonic composition. In some embodiments, the first chromophore is present in an amount of 0.0001%-2%, 0.001%-3%, 0.001%-0.01%, 0.005%-0.1%, 0.1%-0.5%, 0.5%-2%, 1%-5%, 2.5%-7.5%, 5%-10%, 7.5%-12.5%, 10%-15%, 12.5%-17.5%, 15%-20%, 17.5%-22.5%, 20%-25%, 22.5%-27.5%, 25%-30%, 27.5%-32.5%, 30%-35%, 32.5%-37.5%, or 35%-40% by weight of the biophotonic composition. In certain embodiments, the at least second chromophore is present in an amount of about 0.0001%-2%, about 0.001%-3%, about 0.001%-0.01%, about 0.005%-0.1%, about 0.1%-0.5%, about 0.5%-2%, about 1%-5%, about 2.5%-7.5%, about 5%-10%, about 7.5%-12.5%, about 10%-15%, about 12.5%-17.5%, about 15%-20%, about 17.5%-22.5%, about 20%-25%, about 22.5%-27.5%, about 25%-30%, about 27.5%-32.5%, about 30%-35%, about 32.5%-37.5%, or about 35%-40% by weight of the biophotonic composition. In some embodiments, the at least second chromophore is present in an amount of 0.0001%-2%, 0.001%-3%, 0.001%-0.01%, 0.005%-0.1%, 0.1%-0.5%, 0.5%-2%, 1%-5%, 2.5%-7.5%, 5%-10%, 7.5%-12.5%, 10%-15%, 12.5%-17.5%, 15%-20%, 17.5%-22.5%, 20%-25%, 22.5%-27.5%, 25%-30%, 27.5%-32.5%, 30%-35%, 32.5%-37.5%, or 35%-40% by weight of the biophotonic composition. In certain embodiments, the third chromophore is present in an amount of about 0.0001%-2%, about 0.001%-3%, about 0.001%-0.01%, about 0.005%-0.1%, about 0.1%-0.5%, about 0.5%-2%, about 1%-5%, about 2.5%-7.5%, about 5%-10%, about 7.5%-12.5%, about 10%-15%, about 12.5%-17.5%, about 15%-20%, about 17.5%-22.5%, about 20%-25%, about 22.5%-27.5%, about 25%-30%, about 27.5%-32.5%, about 30%-35%, about 32.5%-37.5%, or about 35%-40% by weight of the biophotonic composition. In certain embodiments, the third chromophore is present in an amount of 0.0001%-2%, 0.001%-3%, 0.001%-0.01%, 0.005%-0.1%, 0.1%-0.5%, 0.5%-2%, 1%-5%, 2.5%-7.5%, 5%-10%, 7.5%-12.5%, 10%-15%, 12.5%-17.5%, 15%-20%, 17.5%-22.5%, 20%-25%, 22.5%-27.5%, 25%-30%, 27.5%-32.5%, 30%-35%, 32.5%-37.5%, or 35%-40% by weight of the biophotonic composition. In certain embodiments, the total weight of chromophore or combination of chromophores may be in the amount of about 0.0001%-2%, about 0.005%-1%, about 0.05%-2%, about 1%-5%, about 2.5%-7.5%, about 5%-10%, about 7.5%-12.5%, about 10%-15%, about 12.5%-17.5%, about 15%-20%, about 17.5%-22.5%, about 20%-25%, about 22.5%-27.5%, about 25%-30%, about 27.5%-32.5%, about 30%-35%, about 32.5%-37.5%, or about 35%-40.0% by weight of the biophotonic composition. In some embodiments, the total weight of chromophore or combination of chromophores may be in the amount of 0.0001%-2%, 0.005%-1%, 0.05%-2%, 1%-5%, 2.5%-7.5%, 5%-10%, 7.5%-12.5%, 10%-15%, 12.5%-17.5%, 15%-20%, 17.5%-22.5%, 20%-25%, 22.5%-27.5%, 25%-30%, 27.5%-32.5%, 30%-35%, 32.5%-37.5%, or 35%-40.0% by weight of the biophotonic composition. In certain embodiments, the total weight of chromophore or combination of chromophores may be in the amount of about 0.005%-1% by weight of the biophotonic composition, such as 0.005%-1% by weight of the biophotonic composition. In certain embodiments, the total weight of chromophore or combination of chromophores may be in the amount of about 0.05%-2% by weight of the biophotonic composition, such as 0.05%-2% by weight of the biophotonic composition. In certain embodiments, the total weight of chromophore or combination of chromophores may be in the amount of about 1%-5% by weight of the biophotonic composition, such as 1%-5% by weight of the biophotonic composition. In certain embodiments, the total weight of chromophore or combination of chromophores may be in the amount of about 2.5%-7.5% by weight of the biophotonic composition, such as 2.5%-7.5% by weight of the biophotonic composition. In certain embodiments, the total weight of chromophore or combination of chromophores may be in the amount of about 5%-10% by weight of the biophotonic composition, such as 5%-10% by weight of the biophotonic composition.

The concentration of the chromophore(s) to be used can be selected based on the desired intensity and duration of the biophotonic activity from the biophotonic composition, and on the desired medical or cosmetic effect. For example, some dyes such as xanthene dyes reach a ‘saturation concentration’ after which further increases in concentration do not provide substantially higher emitted fluorescence. Further increasing the chromophore(s) concentration above the saturation concentration can reduce the amount of activating light passing through the matrix. Therefore, if more fluorescence is required for a certain application than activating light, a high concentration of chromophore can be used. However, if a balance is required between the emitted fluorescence and the activating light, a concentration close to or lower than the saturation concentration can be chosen.

Suitable additional chromophores (synthetic or derived from natural source) that may be included in the biophotonic compositions of the present disclosure include, but are not limited to the following:

Chlorophyll Dyes

Exemplary chlorophyll dyes that are useful in the compositions, methods, and uses of the disclosure, include but are not limited to chlorophyll a, chlorophyll b, oil soluble chlorophyll, bacteriochlorophyll a, bacteriochlorophyll b, bacteriochlorophyll c, bacteriochlorophyll d, protochlorophyll, protochlorophyll a, amphiphilic chlorophyll derivative 1, and amphiphilic chlorophyll derivative 2.

Xanthene Derivatives

Exemplary xanthene dyes that are useful in the compositions, methods, and uses of the disclosure include, but are not limited to, Eosin B, Eosin B (4′,5′-dibromo,2′,7′-dinitro-fluorescein, dianion), Eosin Y, Eosin Y (2′,4′,5′,7′-tetrabromo-fluorescein, dianion), Eosin (2′,4′,5′,7′-tetrabromo-fluorescein, dianion), Eosin (2′,4′,5′,7′-tetrabromo-fluorescein, dianion) methyl ester, Eosin (2′,4′,5′,7′-tetrabromo-fluorescein, monoanion) p-isopropylbenzyl ester, Eosin derivative (2′,7′-dibromo-fluorescein, dianion), Eosin derivative (4′,5′-dibromo-fluorescein, dianion), Eosin derivative (2′,7′-dichloro-fluorescein, dianion), Eosin derivative (4′,5′-dichloro-fluorescein, dianion); Eosin derivative (2′,7′-diiodo-fluorescein, dianion), Eosin derivative (4′,5′-diiodo-fluorescein, dianion), Eosin derivative (tribromo-fluorescein, dianion), Eosin derivative (2′,4′,5′,7′-tetrachloro-fluorescein, dianion), Eosin; Eosin dicetylpyridinium chloride ion pair, Erythrosin B (2′,4′,5′,7′-tetraiodo-fluorescein, dianion), Erythrosine, Erythrosin dianion, Erythiosin B, Fluorescein, Fluorescein dianion, Phloxin B (2′,4′,5′,7′-tetrabromo-3,4,5,6-tetrachloro-fluorescein, dianion), Phloxin B (tetrachloro-tetrabromo-fluorescein), Phloxine B, Rose Bengal (3,4,5,6-tetrachloro-2′,4′,5′,7′-tetraiodofluorescein, dianion), Pyronin G, Pyronin J, Pyronin Y. Additional xanthene dyes that are useful in the compositions, methods, and uses of the disclosure also include, but are not limited to, Rhodamine dyes such as 4,5-dibromo-rhodamine methyl ester; 4,5-dibromo-rhodamine n-butyl ester; Rhodamine 101 methyl ester; Rhodamine 123; Rhodamine 6G; Rhodamine 6G hexyl ester; tetrabromo-rhodamine 123; and tetramethyl-rhodamine ethyl ester.

In some embodiments of the disclosure, the xanthene chromophore is selected from Eosin, Eosin Y, Eosin B, Erythrosin B, Fluorescein, Rose Bengal, Phloxin B, or combinations thereof. In some embodiments of the disclosure, the xanthene chromophore is selected from Eosin Y, Eosin B, Erythrosin B, Fluorescein, Rose Bengal, Phloxin B, or combinations thereof. In some embodiments of the disclosure, the xanthene chromophore is Eosin. In some embodiments of the disclosure, the xanthene chromophore is Eosin B. In some embodiments of the disclosure, the xanthene chromophore is Eosin Y. In some embodiments of the disclosure, the xanthene chromophore is Erythrosin B. In some embodiments of the disclosure, the xanthene chromophore is Fluorescein. In some embodiments of the disclosure, the xanthene chromophore is Rose Bengal. In some embodiments of the disclosure, the xanthene chromophore is Phloxin B. In some embodiments the xanthene chromophore is a combination of Rose Bengal and Eosin Y.

Methylene Blue Dyes

Exemplary methylene blue derivatives that are useful in the compositions, methods, and uses of the disclosure include, but are not limited to, 1-methyl methylene blue; 1,9-dimethyl methylene blue; methylene blue; methylene blue (16 μM); methylene blue (14 μM); methylene violet; bromomethylene violet; 4-iodomethylene violet; 1,9-dimethyl-3-dimethyl-amino-7-diethyl-amino-phenothiazine; and 1,9-dimethyl-3-diethylamino-7-dibutyl-amino-phenothiazine.

Azo Dyes

Exemplary azo (or diazo-) dyes that are useful in the compositions, methods, and uses of the disclosure include, but are not limited to, methyl violet, neutral red, para red (pigment red 1), amaranth (Azorubine S), Carmoisine (azorubine, food red 3, acid red 14), allura red AC (FD&C 40), tartrazine (FD&C Yellow 5), orange G (acid orange 10), Ponceau 4R (food red 7), methyl red (acid red 2), and murexide-ammonium purpurate.

In some embodiments of the disclosure, the one or more chromophores that are useful in the compositions, methods, and uses of the disclosure include, but are not limited to, Acid black 1, Acid blue 22, Acid blue 93, Acid fuchsin, Acid green, Acid green 1, Acid green 5, Acid magenta, Acid orange 10, Acid red 26, Acid red 29, Acid red 44, Acid red 51, Acid red 66, Acid red 87, Acid red 91, Acid red 92, Acid red 94, Acid red 101, Acid red 103, Acid roseine, Acid rubin, Acid violet 19, Acid yellow 1, Acid yellow 9, Acid yellow 23, Acid yellow 24, Acid yellow 36, Acid yellow 73, Acid yellow S, Acridine orange, Acriflavine, Alcian blue, Alcian yellow, Alcohol soluble eosin, Alizarin, Alizarin blue 2RC, Alizarin carmine, Alizarin cyanin BBS, Alizarol cyanin R, Alizarin red S, Alizarin purpurin, Aluminon, Amido black 10B, Amidoschwarz, Aniline blue WS, Anthracene blue SWR, Auramine O, Azocannine B, Azocarmine G, Azoic diazo 5, Azoic diazo 48, Azure A, Azure B, Azure C, Basic blue 8, Basic blue 9, Basic blue 12, Basic blue 15, Basic blue 17, Basic blue 20, Basic blue 26, Basic brown 1, Basic fuchsin, Basic green 4, Basic orange 14, Basic red 2 (Saffranin 0), Basic red 5, Basic red 9, Basic violet 2, Basic violet 3, Basic violet 4, Basic violet 10, Basic violet 14, Basic yellow 1, Basic yellow 2, Biebrich scarlet, Bismarck brown Y, Brilliant crystal scarlet 6R, Calcium red, Carmine, Carminic acid (acid red 4), Celestine blue B, China blue, Cochineal, Celestine blue, Chrome violet CG, Chromotrope 2R, Chromoxane cyanin R, Congo corinth, Congo red, Cotton blue, Cotton red, Croceine scarlet, Crocin, Crystal ponceau 6R, Crystal violet, Dahlia, Diamond green B, DiOC6, Direct blue 14, Direct blue 58, Direct red, Direct red 10, Direct red 28, Direct red 80, Direct yellow 7, Eosin B, Eosin Bluish, Eosin, Eosin Y, Eosin yellowish, Eosinol, Erie garnet B, Eriochrome cyanin R, Erythrosin B, Ethyl eosin, Ethyl green, Ethyl violet, Evans blue, Fast blue B, Fast green FCF, Fast red B, Fast yellow, Fluorescein, Food green 3, Gallein, Gallamine blue, Gallocyanin, Gentian violet, Haematein, Haematine, Haematoxylin, Helio fast rubin BBL, Helvetia blue, Hematein, Hematine, Hematoxylin, Hoffman's violet, Imperial red, Indocyanin green, Ingrain blue, Ingrain blue 1, Ingrain yellow 1, INT, Kermes, Kermesic acid, Kernechtrot, Lac, Laccaic acid, Lauth's violet, Light green, Lissamine green SF, Luxol fast blue, Magenta 0, Magenta I, Magenta II, Magenta III, Malachite green, Manchester brown, Martius yellow, Merbromin, Mercurochrome, Metanil yellow, Methylene azure A, Methylene azure B, Methylene azure C, Methylene blue, Methyl blue, Methyl green, Methyl violet, Methyl violet 2B, Methyl violet 10B, Mordant blue 3, Mordant blue 10, Mordant blue 14, Mordant blue 23, Mordant blue 32, Mordant blue 45, Mordant red 3, Mordant red 11, Mordant violet 25, Mordant violet 39 Naphthol blue black, Naphthol green B, Naphthol yellow S, Natural black 1, Natural red, Natural red 3, Natural red 4, Natural red 8, Natural red 16, Natural red 25, Natural red 28, Natural yellow 6, NBT, Neutral red, New fuchsin, Niagara blue 3B, Night blue, Nile blue, Nile blue A, Nile blue oxazone, Nile blue sulphate, Nile red, Nitro BT, Nitro blue tetrazolium, Nuclear fast red, Oil red O, Orange G, Orcein, Pararosanilin, Phloxine B, phycobilins, Phycocyanins, Phycoerythrins. Phycoerythrincyanin (PEC), Phthalocyanines, Picric acid, Ponceau 2R, Ponceau 6R, Ponceau B, Ponceau de Xylidine, Ponceau S, Primula, Purpurin, Pyronin B, Pyronin G, Pyronin Y, Rhodamine B, Rosanilin, Rose bengal, Saffron, Safranin O, Scarlet R, Scarlet red, Scharlach R, Shellac, Sirius red F3B, Solochrome cyanin R, Soluble blue, Solvent black 3, Solvent blue 38, Solvent red 23, Solvent red 24, Solvent red 27, Solvent red 45, Solvent yellow 94, Spirit soluble eosin, Sudan III, Sudan IV, Sudan black B, Sulfur yellow S, Swiss blue, Tartrazine, Thioflavine S, Thioflavine T, Thionin, Toluidine blue, Toluyline red, Tropaeolin G, Trypaflavine, Trypan blue, Uranin, Victoria blue 4R, Victoria blue B, Victoria green B, Water blue I, Water soluble eosin, Xylidine ponceau, or Yellowish eosin.

In some embodiments of the disclosure, the first chromophore is selected from Eosin, Eosin Y, Eosin B, Erythrosin B, Fluorescein, Rose Bengal, Phloxin B, or combinations thereof. In some embodiments of the disclosure, the first chromophore is selected from Eosin Y, Eosin B, Erythrosin B, Fluorescein, Rose Bengal, Phloxin B, or combinations thereof. In some embodiments of the disclosure, the first chromophore is Eosin. In some embodiments of the disclosure, the first chromophore is Eosin B. In some embodiments of the disclosure, the first chromophore is Eosin Y. In some embodiments of the disclosure, the first chromophore is Erythrosin B. In some embodiments of the disclosure, the first chromophore is Fluorescein. In some embodiments of the disclosure, the first chromophore is Rose Bengal. In some embodiments of the disclosure, the first chromophore is Phloxin B. In some embodiments of the disclosure, the first chromophore is a combination of Eosin Y and Rose Bengal.

In some embodiments of the disclosure, the at least second chromophore is selected from Eosin, Eosin Y, Eosin B, Erythrosin B, Fluorescein, Rose Bengal, Phloxin B, or combinations thereof. In some embodiments of the disclosure, the at least second chromophore is selected from Eosin Y, Eosin B, Erythrosin B, Fluorescein, Rose Bengal, Phloxin B, or combinations thereof. In some embodiments of the disclosure, the at least second chromophore is Eosin. In some embodiments of the disclosure, the at least second chromophore is Eosin B. In some embodiments of the disclosure, the at least second chromophore is Eosin Y. In some embodiments of the disclosure, the at least second chromophore is Erythrosin B. In some embodiments of the disclosure, the at least second chromophore is Fluorescein. In some embodiments of the disclosure, the at least second chromophore is Rose Bengal. In some embodiments of the disclosure, the at least second chromophore is Phloxin B. In some embodiments of the disclosure, the at least second chromophore is a combination of Eosin Y and Rose Bengal.

In certain embodiments, the biophotonic composition of the present disclosure includes any of the chromophores listed above, or a combination thereof, so as to provide a synergistic biophotonic effect at the application site.

Without being bound to any particular theory, a synergistic effect of the chromophore combinations means that the biophotonic effect is greater than the sum of their individual effects. Advantageously, this may translate to increased reactivity of the biophotonic composition, faster or improved treatment time. Also, the treatment conditions need not be altered to achieve the same or better treatment results, such as time of exposure to light, power of light source used, and wavelength of light used. In other words, use of synergistic combinations of chromophores may allow the same or better treatment without necessitating a longer time of exposure to a light source, a higher power light source or a light source with different wavelengths.

By means of synergistic effects of the chromophore combinations in the composition, chromophores which cannot normally be activated by an activating light (such as a blue light from an LED) can be activated through energy transfer from chromophores which are activated by the activating light. In this way, the different properties of photoactivated chromophores can be harnessed and tailored according to the cosmetic or the medical therapy required.

In some embodiments, the chromophore or chromophores are selected such that their emitted fluorescent light, on photoactivation, is within one or more of the green, yellow, orange, red and infrared portions of the electromagnetic spectrum, for example having a peak wavelength within the range of about 490 nm to about 800 nm. In certain embodiments, the emitted fluorescent light has a power density of between about 0.005 mW/cm² to about 10 mW/cm², about 0.5 mW/cm² to about 5 mW/cm².

(b) Oxidants

In some embodiments, the biophotonic compositions, methods, and uses of the present disclosure comprise one or more oxidants as a source of oxygen radicals or singlet oxygen. Peroxide compounds are oxidants that contain the peroxy group (R—O—O—R), which is a chainlike structure containing two oxygen atoms, each of which is bonded to the other and a radical or some element. When a biophotonic composition of the present disclosure comprising an oxidant is illuminated with light, the chromophores are excited to a higher energy state. When the chromophores' electrons return to a lower energy state, they emit photons with a lower energy level, thus causing the emission of light of a longer wavelength (Stokes' shift). In the proper environment, some of this energy is transferred to oxygen or the reactive hydrogen peroxide and causes the formation of oxygen radicals, such as singlet oxygen. The singlet oxygen and other reactive oxygen species generated by the activation of the biophotonic composition are thought to operate in a hormetic fashion. That is, a health beneficial effect that is brought about by the low exposure to a normally toxic stimuli (e.g. reactive oxygen), by stimulating and modulating stress response pathways in cells of the targeted tissues. Endogenous response to exogenous generated free radicals (reactive oxygen species) is modulated in increased defense capacity against the exogenous free radicals and induces acceleration of healing and regenerative processes. Furthermore, the extreme sensitivity of bacteria to exposure to free radicals makes the biophotonic composition of the present disclosure potentially a bactericidal composition.

Peroxide compounds are oxidants that contain the peroxy group (R—O—O—R), which is a chainlike structure containing two oxygen atoms, each of which is bonded to the other and a radical or some element. Suitable oxidants for preparation of the active medium include, but are not limited to:

Hydrogen peroxide (H₂O₂) is a powerful oxidizing agent, and breaks down into water and oxygen and does not form any persistent, toxic residual compound. A suitable range of concentration over which hydrogen peroxide can be used in the biophotonic composition is from about 0.01% to about 30%, about 1% to about 25%, about 5% to about 20%, about 10% to about 15%, or less than about 20% by weight of the total composition. In some embodiments, hydrogen peroxide is present in an amount from about 0.1% to about 12%, from about 1% to about 12%, from about 3.5% to about 12%, from about 3.5% to about 6% or from about 0.1% to about 6% by weight of the total composition. In some embodiments, hydrogen peroxide is present in an amount from 0.01% to 30%, 1% to 25%, 5% to 20%, 10% to 15%, or less than 20% by weight of the total composition. In some embodiments, hydrogen peroxide is present in an amount from 0.1% to 12%, from 1% to 12%, from 3.5% to 12%, from 3.5% to 6% or from 0.1% to 6% by weight of the total composition.

Urea hydrogen peroxide (also known as urea peroxide, carbamide peroxide or percarbamide) is soluble in water and contains approximately 35% hydrogen peroxide. Urea peroxide brakes down to urea and hydrogen peroxide in a slow-release fashion that can be accelerated with heat or photochemical reactions. The released urea ((NH₂)₂CO₂), is highly soluble in water and is a powerful protein denaturant. It increases solubility of some proteins and enhances rehydration of the skin and/or mucosa. A suitable range of concentration over which urea peroxide can be used in the biophotonic composition of the present disclosure is less than about 25%, or less than about 20%, or less than about 15%, or less than about 10%, or less than about 5%, or from about 0.1% to about 5%, or from about 1% to about 15% by weight of the total composition. In some embodiments, urea peroxide is present in less than 25%, or less than 20%, or less than 15%, or less than 10%, or less than 5%, or from 0.1 to 5%, or from 1% to 15% by weight of the total composition. In some embodiments, urea peroxide is present in an amount from about 0.3% to about 36%, from about 3% to about 36%, or from about 10% to about 36%, or from about 3% to about 16% or from about 0.3% to about 16% by weight of the total composition. In some embodiments, urea peroxide is present in an amount from 0.3% to 36%, from 3% to 36%, or from 10% to 36%, or from 3% to 16% or from 0.3% to 16% by weight of the total composition. In some embodiments, urea peroxide is present in an amount of about 2% by weight of the total composition, such as 2% by weight of the total composition. In some embodiments, urea peroxide is present in an amount of about 3% by weight of the total composition, such as 3% by weight of the total composition. In some embodiments, urea peroxide is present in an amount of about 6% by weight of the total composition, such as 6% by weight of the total composition. In some embodiments, urea peroxide is present in an amount of about 8% by weight of the total composition, such as 8% by weight of the total composition. In some embodiments, urea peroxide is present in an amount of about 12% by weight of the total composition, such as 12% by weight of the total composition.

Benzoyl peroxide consists of two benzoyl groups (benzoic acid with the H of the carboxylic acid removed) joined by a peroxide group. It is found in treatments for acne, in concentrations varying from 2.5% to 10%. The released peroxide groups are effective at killing bacteria. Benzoyl peroxide also promotes skin turnover and clearing of pores, which further contributes to decreasing bacterial counts and reduce acne. Benzoyl peroxide breaks down to benzoic acid and oxygen upon contact with skin, neither of which is toxic. A suitable range of concentration over which benzoyl peroxide can be used in the biophotonic composition is from about 2.5% to about 20%, or about 2.5% to about 10% by weight of the total composition. In some embodiments, benzoyl peroxide is present in an amount from 2.5% to 20%, or 2.5% to 10% by weight of the total composition. In some embodiments, benzoyl peroxide is present in an amount from about 1% to about 10%, or from about 1% to about 8%, or from about 2.5% to about 5% by weight of the total composition. In some embodiments, benzoyl peroxide is present in an amount from 1% to 10%, or from 1% to 8%, or from 2.5% to 5% by weight of the total composition.

In some embodiments, the peroxide or peroxide precursor is a peroxy acid, an alkali metal peroxide, an alkali metal percarbonate, a peroxyacetic acid, an alkali metal perborate, or methyl ethyl ketone peroxide. In some embodiments, the oxidant is methyl ethyl ketone peroxide. A suitable range of concentration over which methyl ethyl ketone peroxide can be used in the biophotonic composition is from about 0.01% to about 15% by weight of the total composition, such as 0.01% to 15% by weight of the total composition.

(c) Carrier Medium

In some embodiments, the biophotonic compositions, methods, and uses of the present disclosure comprise a carrier medium made from one or more thickening agents. Thickening agents are present in an amount and ratio sufficient to provide a desired viscosity, flexibility, rigidity, tensile strength, tear strength, elasticity, and adhesiveness. The thickening agents are selected so that the chromophore(s) can remain photoactive in the carrier medium. The thickening agents are also selected according to the optical transparency of the carrier medium. The carrier medium should be able to transmit sufficient light to activate the at least one chromophore and, in embodiments where fluorescence is emitted by the activated chromophore, the carrier medium should also be able to transmit the emitted fluorescent light to tissues. It will be recognized by persons skilled in the art that the thickening agent is an appropriate medium for the chromophore(s) selected. For example, the inventors have noted that some xanthene dyes do not fluoresce in non-hydrated media, so hydrated polymers or polar solvents may be used. The thickening agents should also be selected according to the intended use. For example, if the biophotonic composition is to be applied onto tissue, the carrier medium is preferably biocompatible, or the carrier medium has an outside layer of a biocompatible composition which will interface the tissue.

Thickening Agents

In some embodiments, the content of a thickening agent is present in the composition in an amount of from about 0.001% to about 40% (w/w %) of the total weight. In certain embodiments, the total content of the thickening agent is about 0.001%-0.01%, about 0.005%-0.05%, about 0.01%-0.1, about 0.05%-0.5%, about 0.1%-1%, about 0.5%-5%, about 1%-5%, about 2.5%-7.5%, about 5%-10%, about 7.5%-12.5%, about 10%-15%, about 12.5%-17.5%, about 15%-20%, about 15%-25%, about 20%-30%, about 25%-35%, or about 30%-40% by weight of the total composition. In some embodiments, the total content of the thickening agent is 0.001%-0.01%, 0.005%-0.05%, 0.01%-0.1, 0.05%-0.5%, 0.1%-1%, 0.5%-5%, 1%-5%, 2.5%-7.5%, 5%-10%, 7.5%-12.5%, 10%-15%, 12.5%-17.5%, 15%-20%, 15%-25%, 20%-30%, 25%-35%, or 30%-40% by weight of the total composition. It will be recognized by one of skill in the art that the viscosity, flexibility, rigidity, tensile strength, tear strength, elasticity, and adhesiveness can be adjusted by varying the content of the thickening agent. Methods of determining viscosity, flexibility, rigidity, tensile strength, tear strength, elasticity, and adhesiveness are known in the art.

Thickening agents that can be used to prepare the biophotonic compositions of the present disclosure include but are not limited to a hydrophilic polymer, a hygroscopic polymer or a hydrated polymer. The thickening agent may be polyanionic in charge character. The thickening agent may comprise carboxylic functional groups, and may further contain 2 to 7 carbon atoms per functional group. The thickening agents may include polymers, copolymers, and monomers of: vinylpyrrolidones, methacrylamides, acrylamides N-vinylimidazoles, carboxy vinyls, vinyl esters, vinyl ethers, silicones, polyethyleneoxides, polyethyleneglycols, vinylalcohols, sodium acrylates, acrylates, maleic acids, N,N-dimethylacrylamides, diacetone acrylamides, acrylamides, acryloyl morpholine, pluronic, collagens, polyacrylamides, polyacrylates, polyvinyl alcohols, polyvinylenes, polyvinyl silicates, polyacrylates substituted with a sugar (e.g., sucrose, glucose, glucosamines, galactose, trehalose, mannose, or lactose), acylamidopropane sulfonic acids, tetramethoxyorthosilicates, methyltrimethoxyorthosilicates, tetraalkoxyorthosilicates, trialkoxyorthosilicates, glycols, propylene glycol, glycerine, polysaccharides, alginates, dextrans, cyclodextrin, celluloses, modified celluloses, oxidized celluloses, chitosans, chitins, guars, carrageenans, hyaluronic acids, inulin, starches, modified starches, agarose, methylcelluloses, plant gums, hyaluronans, hydrogels, gelatins, glycosaminoglycans, carboxymethyl celluloses, hydroxyethyl celluloses, hydroxy propyl methyl celluloses, pectins, low-methoxy pectins, cross-linked dextrans, starch-acrylonitrile graft copolymers, starch sodium polyacrylate, hydroxyethyl methacrylates, hydroxyl ethyl acrylates, polyvinylene, polyethylvinylethers, polymethyl methacrylates, polystyrenes, polyurethanes, polyalkanoates, polylactic acids, polylactates, poly(3-hydroxybutyrate), sulfonated hydrogels, AMPS (2-acrylamido-2-methyl-1-propanesulfonic acid), SEM (sulfoethylmethacrylate), SPM (sulfopropyl methacrylate), SPA (sulfopropyl acrylate), N,N-dimethyl-N-methacryloxyethyl-N-(3-sulfopropyl)ammonium betaine, methacryllic acid amidopropyl-dimethyl ammonium sulfobetaine, SPI (itaconic acid-bis(1-propylsulfonizacid-3)ester di-potassium salt), itaconic acids, AMBC (3-acrylamido-3-methylbutanoic acid), beta-carboxyethyl acrylate (acrylic acid dimers), and maleic anhydride-methylvinyl ether polymers, derivatives thereof, salts thereof, acids thereof, and combinations thereof. In some embodiments, the thickening agent comprises 2-Hydroxyethyl methacrylate (HEMA) either alone or in addition to another thickening agent. In some embodiments, the 2-Hydroxyethyl methacrylate (HEMA) is added in a form such as microspheres or in a further physically reduced form such as a finely ground particulate form or in a pulverized, powder form.

In certain embodiments, the at least one thickening agent is a synthetic polymer selected from one or more of vinyl polymers, polyoxythylene-polyoxypropylene copolymers, poly(ethylene oxide), acrylamide polymers and derivatives and salts thereof. In a further embodiment the vinyl polymer is selected from one or more of polyacrylic acid, polymethacrylic acid, polyvinyl pyrrolidone, and polyvinyl alcohol. In other embodiments, the vinyl polymer is a carboxy vinyl polymer or a carbomer obtained by the polymerization of acrylic acid. The carboxy vinyl polymer or carbomer may be cross-linked.

As mentioned above, in some embodiments, the at least one thickening agent of the carrier medium is one or more carbomers. Carbomers are synthetic high molecular weight polymers of acrylic acid that are crosslinked with either allylsucrose or allylethers of pentaerythritol having a molecular weight of about 3×10⁶. The gelation mechanism depends on neutralization of the carboxylic acid moiety to form a soluble salt. The polymer is hydrophilic and produces sparkling clear gels when neutralized. Carbomers are available as fine white powders which disperse in water to form acidic colloidal suspensions (a 1% dispersion has approximately pH 3) of low viscosity. Neutralization of these suspensions using a base, for example sodium, potassium or ammonium hydroxides, low molecular weight amines and alkanolamines, results in the formation of clear translucent gels.

In some embodiments, the carbomer is a Carbopol®. Such polymers are commercially available from B.F. Goodrich or Lubrizol under the designation Carbopol® 71G NF, 420, 430, 475, 488, 493, 910, 934, 934P, 940, 971PNF, 974P NF, 980 NF, 981 NF and the like. Carbopols are versatile controlled-release polymers, as described by Brock (Pharmacotherapy, 14:430-7 (1994), incorporated herein by reference) and Durrani (Pharmaceutical Res. (Supp.) 8:S-135 (1991), incorporated herein by reference), and belong to a family of carbomers which are synthetic, high molecular weight, non-linear polymers of acrylic acid, cross-linked with polyalkenyl polyether. In certain embodiments, the carbomer is Carbopol® 940, Carbopol® 980, ETD 2020NF, Carbopol® 1382, 71G NF, 971P NF, 974P NF, 980 NF, 981 NF, 5984 EP, ETF 2020 NF, Ultrez 10 NF, Ultrez 20, Ultrez 21, 1342 NF, 934 NF, 934P NF, 940 NF or 941 NF, or combinations thereof. In some embodiments, the carbomer is cross-linked with alkyl acrylate or allyl pentaerythritol. In some embodiments, the carbomer is present in the composition in an amount of from about 0.01 wt % to about 15 wt %, or about 0.05 wt % to about 5 wt %, or about 0.5 wt % to about 2 wt %. In some embodiments, the carbomer is present in the composition in an amount of from 0.01 wt % to 15 wt %, or 0.05 wt % to 5 wt %, or 0.5 wt % to 2 wt %.

In certain embodiments, the at least one thickening agent of the carrier medium is a glycol, such as ethylene glycol or propylene glycol. In further embodiments, the at least one thickening agent of the carrier medium is a poly (ethylene oxide) polymer (such as POLYOX from Dow Chemical), linear PVP and cross-linked PVP, PEG/PPG copolymers (such as BASF Pluracare L1220), ethylene oxide (EO)-propylene oxide (PO) block copolymers (such as polymers sold under the trade mark Pluronic available from BASF Corporation), ester gum, shellac, pressure sensitive silicone adhesives (such as BioPSA from Dow-Corning), or mixtures thereof. In some embodiments, a copolymer comprises (PVM/MA). In some embodiments, a copolymer comprises poly (methylvinylether/maleic anhydride). In some embodiments, a copolymer comprises poly (methylvinylether/maleic acid). In some embodiments, a copolymer comprises poly (methylvinylether/maleic acid) half esters. In some embodiments, a copolymer comprises poly (methylvinylether/maleic acid) mixed salts.

In certain embodiments of the disclosure, the at least one thickening agent of the carrier medium is a protein-based polymer. Such protein-based polymer may be selected from at least one of gelatin or collagen. For example, the composition may comprise at least about 4 wt %, about 4 wt % to about 25 wt %, or about 10 wt % to about 20 wt % gelatin within the biophotonic composition. In some embodiments, the composition may comprise at least 4 wt %, 4 wt % to 25 wt %, or 10 wt % to 20 wt % gelatin within the biophotonic composition. The composition may comprise at least about 5 wt %, about 5 wt % to about 25 wt %, or about 10 wt % to about 20 wt % collagen and/or sodium hyaluronate within the biophotonic composition. In some embodiments, the composition may comprise at least 5 wt %, 5 wt % to 25 wt %, or 10 wt % to 20 wt % collagen and/or sodium hyaluronate within the biophotonic composition. Alternatively, a lower weight percentage of protein-based polymers may be used together with chemical cross-linkers or any other cross-linking means.

In certain embodiments of the disclosure, the at least one thickening agent of the carrier medium is sodium hyaluronate. For example, the composition may comprise at least about 4 wt %, about 4 wt % to about 25 wt %, or about 10 wt % to about 20 wt % sodium hyaluronate within the biophotonic composition. In some embodiments, the composition may comprise at least 4 wt %, 4 wt % to 25 wt %, or 10 wt % to 20 wt % sodium hyaluronate within the biophotonic composition. Alternatively, a lower weight percentage of sodium hyaluronate may be used together with chemical cross-linkers or any other cross-linking means.

In certain embodiments of the disclosure, the at least one thickening agent of the carrier medium is a polysaccharide, which may be from at least one of starch, chitosan, chitin, agar, alginates, xanthan, carrageenan, guar gum, gellan gum, pectin, or locust bean gum.

The biophotonic composition of the present disclosure may optionally be provided with a water-insoluble substrate. By “water insoluble”, it is meant that the substrate does not dissolve in or readily break apart upon immersion in water. In some embodiments, the water-insoluble substrate is the implement or vehicle for delivering the treatment composition to the skin or target tissue. A wide variety of substances can be used as the water-insoluble substrate. One or more of the following non-limiting characteristics may be desirable: (i) sufficient wet strength for use, (ii) sufficient softness, (iii) sufficient thickness, (iv) appropriate size, (v) air permeability, and (vi) hydrophilicity.

Non-limiting examples of suitable water-insoluble substrates which meet the above criteria include nonwoven substrates, woven substrates, hydroentangled substrates, air entangled substrates, natural sponges, synthetic sponges, polymeric netted meshes, and the like. Some embodiments employ nonwoven substrates since they are economical and readily available.

By “nonwoven”, it is meant that the layer is comprised of fibers which are not woven into a fabric but rather are formed into a sheet, mat, or pad layer.

(d) Antimicrobials

According to some embodiments, the biophotonic compositions of the methods and uses of the present disclosure may optionally further comprise one or more antimicrobials. Antimicrobials kill microbes or inhibit their growth or accumulation. Exemplary antimicrobials (or antimicrobial agent) are recited in U.S. Patent Application Publication Nos. 20040009227 and 20110081530, the disclosures of both of which are herein incorporated by reference. Suitable antimicrobials for use in the methods of the present disclosure include, but not limited to, phenolic and chlorinated phenolic and chlorinated phenolic compounds, resorcinol and its derivatives, bisphenolic compounds, benzoic esters (parabens), halogenated carbonilides, polymeric antimicrobial agents, thazolines, trichloromethylthioimides, natural antimicrobial agents (also referred to as “natural essential oils”), metal salts, and broad-spectrum antibiotics.

Additionally, the biophotonic composition of the present disclosure comprises a peroxide or peroxide derivative, which upon illumination of the biophotonic composition will lead to the generation oxygen radicals. The extreme sensitivity of bacteria to exposure to free radicals makes the biophotonic composition of the present disclosure potentially a bactericidal composition.

Examples of phenolic and chlorinated phenolic antimicrobial agents that can be used in the compositions defined herein include, but are not limited to: phenol; 2-methyl phenol; 3-methyl phenol; 4-methyl phenol; 4-ethyl phenol; 2,4-dimethyl phenol; 2,5-dimethyl phenol; 3,4-dimethyl phenol; 2,6-dimethyl phenol; 4-n-propyl phenol; 4-n-butyl phenol; 4-n-amyl phenol; 4-tert-amyl phenol; 4-n-hexyl phenol; 4-n-heptyl phenol; mono- and poly-alkyl and aromatic halophenols; p-chlorophenyl; methyl p-chlorophenol; ethyl p-chlorophenol; n-propyl p-chlorophenol; n-butyl p-chlorophenol; n-amyl p-chlorophenol; sec-amyl p-chlorophenol; n-hexyl p-chlorophenol; cyclohexyl p-chlorophenol; n-heptyl p-chlorophenol; n-octyl; p-chlorophenol; o-chlorophenol; methyl o-chlorophenol; ethyl o-chlorophenol; n-propyl o-chlorophenol; n-butyl o-chlorophenol; n-amyl o-chlorophenol; tert-amyl o-chlorophenol; n-hexyl o-chlorophenol; n-heptyl o-chlorophenol; o-benzyl p-chlorophenol; o-benxyl-m-methyl p-chlorophenol; o-benzyl-m,m-dimethyl p-chlorophenol; o-phenylethyl p-chlorophenol; o-phenylethyl-m-methyl p-chlorophenol; 3-methyl p-chlorophenol 3,5-dimethyl p-chlorophenol, 6-ethyl-3-methyl p-chlorophenol, 6-n-propyl-3-methyl p-chlorophenol; 6-iso-propyl-3-methyl p-chlorophenol; 2-ethyl-3,5-dimethyl p-chlorophenol; 6-sec-butyl-3-methyl p-chlorophenol; 2-iso-propyl-3,5-dimethyl p-chlorophenol; 6-diethylmethyl-3-methyl p-chlorophenol; 6-iso-propyl-2-ethyl-3-methyl p-chlorophenol; 2-sec-amyl-3,5-dimethyl p-chlorophenol; 2-diethylmethyl-3,5-dimethyl p-chlorophenol; 6-sec-octyl-3-methyl p-chlorophenol; p-chloro-m-cresol p-bromophenol; methyl p-bromophenol; ethyl p-bromophenol; n-propyl p-bromophenol; n-butyl p-bromophenol; n-amyl p-bromophenol; sec-amyl p-bromophenol; n-hexyl p-bromophenol; cyclohexyl p-bromophenol; o-bromophenol; tert-amyl o-bromophenol; n-hexyl o-bromophenol; n-propyl-m,m-dimethyl o-bromophenol; 2-phenyl phenol; 4-chloro-2-methyl phenol; 4-chloro-3-methyl phenol; 4-chloro-3,5-dimethyl phenol; 2,4-dichloro-3,5-dimethylphenol; 3,4,5,6-tetabromo-2-methylphenol; 5-methyl-2-pentylphenol; 4-isopropyl-3-methylphenol; para-chloro-metaxylenol (PCMX); chlorothymol; phenoxyethanol; phenoxyisopropanol; and 5-chloro-2-hydroxydiphenylmethane.

Resorcinol and its derivatives can also be used as antimicrobial agents. Examples of resorcinol derivatives include, but are not limited to: methyl resorcinol; ethyl resorcinol; n-propyl resorcinol; n-butyl resorcinol; n-amyl resorcinol; n-hexyl resorcinol; n-heptyl resorcinol; n-octyl resorcinol; n-nonyl resorcinol; phenyl resorcinol; benzyl resorcinol; phenylethyl resorcinol; phenylpropyl resorcinol; p-chlorobenzyl resorcinol; 5-chloro-2,4-dihydroxydiphenyl methane; 4′-chloro-2,4-dihydroxydiphenyl methane; 5-bromo-2,4-dihydroxydiphenyl methane; and 4′-bromo-2,4-dihydroxydiphenyl methane.

Examples of bisphenolic antimicrobial agents that can be used in the compositions defined herein include, but are not limited to: 2,2′-methylene bis-(4-chlorophenol); 2,4,4′trichloro-2′-hydroxy-diphenyl ether, which is sold by Ciba Geigy, Florham Park, N.J. under the tradename Triclosan®; 2,2′-methylene bis-(3,4,6-trichlorophenol); 2,2′-methylene bis-(4-chloro-6-bromophenol); bis-(2-hydroxy-3,5-dichlorophenyl) sulphide; and bis-(2-hydroxy-5-chlorobenzyl)sulphide.

Examples of benzoic esters (parabens) that can be used in the compositions defined herein include, but are not limited to: methylparaben; propylparaben; butylparaben; ethylparaben; isopropylparaben; isobutylparaben; benzylparaben; sodium methylparaben; and sodium propylparaben.

Examples of halogenated carbanilides that can be used in the compositions defined herein include, but are not limited to: 3,4,4′-trichlorocarbanilides, such as 3-(4-chlorophenyl)-1-(3,4-dichlorphenyl)urea sold under the tradename Triclocarban® by Ciba-Geigy, Florham Park, N.J.; 3-trifluoromethyl-4,4′-dichlorocarbanilide; and 3,3′,4-trichlorocarbanilide.

Examples of polymeric antimicrobial agents that can be used in the compositions defined herein include, but are not limited to: polyhexamethylene biguanide hydrochloride; and poly(iminoimidocarbonyl iminoimidocarbonyl iminohexamethylene hydrochloride), which is sold under the tradename Vantocil® IB.

Examples of thazolines that can be used in the compositions defined herein include, but are not limited to that sold under the tradename Micro-Check® and 2-n-octyl-4-isothiazolin-3-one, which is sold under the tradename Vinyzene® IT-3000 DIDP.

Examples of trichloromethylthioimides that can be used in the compositions as defined herein include, but are not limited to: N-(trichloromethylthio)phthalimide, which is sold under the tradename Fungitrol®; and N-trichloromethylthio-4-cyclohexene-1,2-dicarboximide, which is sold under the tradename Vancide®.

Examples of natural antimicrobial agents that can be used in the compositions as defined herein include, but are not limited to, oils of: anise, lemon, orange, rosemary, wintergreen, thyme, lavender, cloves, hops, tea tree, citronella, wheat, barley, lemongrass, cedar leaf, cedarwood, cinnamon, fleagrass, geranium, sandalwood, violet, cranberry, eucalyptus, vervain, peppermint, gum benzoin, basil, honey, fennel, fir, balsam, menthol, ocmea origanuin, hydastis, carradensis, Berberidaceac daceae, Ratanhiae longa, and Curcuma longa. Also included in this class of natural antimicrobial agents are the key chemical components of the plant oils which have been found to provide antimicrobial benefit. These chemicals include, but are not limited to: anethol, catechole, camphene, thymol, eugenol, eucalyptol, ferulic acid, farnesol, hinokitiol, tropolone, limonene, menthol, methyl salicylate, carvacol, terpineol, verbenone, berberine, ratanhiae extract, caryophellene oxide, citronellic acid, curcumin, nerolidol, and geraniol.

Examples of metal salts that can be used in the compositions include, but are not limited to, salts of metals in groups 3a-5a, 3b-7b, and 8 of the periodic table. Specific examples of metal salts include, but are not limited to, salts of: aluminum, zirconium, zinc, silver, gold, copper, lanthanum, tin, mercury, bismuth, selenium, strontium, scandium, yttrium, cerium, praseodymiun, neodymium, promethum, samarium, europium, gadolinium, terbium, dysprosium, holmium, erbium, thalium, ytterbium, lutetium, and mixtures thereof. An example of the metal-ion based antimicrobial agent is sold under the tradename HealthShield®, and is manufactured by HealthShield Technology, Wakefield, Mass.

Example of broad-spectrum antimicrobial agents that can be used in the compositions as defined herein include, but are not limited to, those that are recited in other categories of antimicrobial agents herein.

Additional antimicrobial agents that can be used in the methods of the disclosure include, but are not limited to: pyrithiones, and in particular pyrithione-including zinc complexes such as that sold under the tradename Octopirox®; dimethyidimethylol hydantoin, which is sold under the tradename Glydant®; methylchloroisothiazolinone/methylisothiazolinone, which is sold under the tradename Kathon CG®; sodium sulfite; sodium bisulfite; imidazolidinyl urea, which is sold under the tradename Germall 115®; diazolidinyl urea, which is sold under the tradename Germall 11®; benzyl alcohol v2-bromo-2-nitropropane-1,3-diol, which is sold under the tradename Bronopol®; formalin or formaldehyde; iodopropenyl butylcarbamate, which is sold under the tradename Polyphase P100®; chloroacetamide; methanamine; methyldibromonitrile glutaronitrile (1,2-dibromo-2,4-dicyanobutane), which is sold under the tradename Tektamer®; glutaraldehyde; 5-bromo-5-nitro-1,3-dioxane, which is sold under the tradename Bronidox®; phenethyl alcohol; o-phenylphenol/sodium o-phenylphenol sodium hydroxymethylglycinate, which is sold under the tradename Suttocide A®; polymethoxy bicyclic oxazolidine; which is sold under the tradename Nuosept C®; dimethoxane; thimersal; dichlorobenzyl alcohol; captan; chlorphenenesin; dichlorophene; chlorbutanol; glyceryl laurate; halogenated diphenyl ethers; 2,4,4′-trichloro-2′-hydroxy-diphenyl ether, which is sold under the tradename Triclosan® and is available from Ciba-Geigy, Florham Park, N.J.; and 2,2′-dihydroxy-5,5′-dibromo-diphenyl ether.

Additional antimicrobial agents that can be used in the methods of the disclosure include those disclosed by U.S. Pat. Nos. 3,141,321; 4,402,959; 4,430,381; 4,533,435; 4,625,026; 4,736,467; 4,855,139; 5,069,907; 5,091,102; 5,639,464; 5,853,883; 5,854,147; 5,894,042; and 5,919,554; and U.S. Pat. Appl. Publ. Nos. 2004/0009227 and 2011/0081530, the contents of all of which are incorporated herein by reference.

(e) Collagens and Agents that Promote Collagen Synthesis

According to some embodiments, the biophotonic compositions, methods and uses of the present disclosure may optionally further comprise one or more collagens and/or agents that promote collagen synthesis. Collagen is a fibrous protein produced in dermal fibroblast cells and forming 70% of the dermis and benefits all stages of the wound healing process. Thus, collagens and agents that promote collagen synthesis may also be useful in the present disclosure. Agents that promote collagen synthesis (i.e., pro-collagen synthesis agents) include amino acids, peptides, proteins, lipids, small chemical molecules, natural products and extracts from natural products.

For instance, it was discovered that intake of vitamin C, iron, and collagen can effectively increase the amount of collagen in skin or bone. See, e.g., U.S. Patent Application Publication No. 20090069217, the contents of which are incorporated herein by reference. Examples of the vitamin C include an ascorbic acid derivative such as L-ascorbic acid or sodium L-ascorbate, an ascorbic acid preparation obtained by coating ascorbic acid with an emulsifier or the like, and a mixture containing two or more of those vitamin Cs at an arbitrary rate. In addition, natural products containing vitamin C such as acerola and lemon may also be used. Examples of the iron preparation include: an inorganic iron such as ferrous sulfate, sodium ferrous citrate, or ferric pyrophosphate; an organic iron such as heme iron, ferritin iron, or lactoferrin iron; and a mixture containing two or more of those irons at an arbitrary rate. In addition, natural products containing iron such as spinach or liver may also be used. Moreover, examples of the collagen include: an extract obtained by treating bone, skin, or the like of a mammal such as bovine or swine with an acid or alkaline; a peptide obtained by hydrolyzing the extract with a protease such as pepsin, trypsin, or chymotrypsin; and a mixture containing two or more of those collagens at an arbitrary rate. Collagens extracted from plant sources may also be used.

Additional pro-collagen synthesis agents are described, for example, in U.S. Pat. Nos. 7,598,291, 7,722,904, 6,203,805, and 5,529,769, and U.S. Patent Application Publication Nos. 20060247313, 20080108681, 20110130459, 20090325885, and 20110086060, the contents of all of which are incorporated herein by reference.

(f) Healing Factors

Healing factors comprise compounds that promote or enhance the healing or regenerative process of the tissues on the application site of the composition. During the photoactivation of the composition, there is an increase of the absorption of molecules at the treatment site. An augmentation in the blood flow at the site of treatment is observed for an extent period of time. An increase in the lymphatic drainage and a possible change in the osmotic equilibrium due to the dynamic interaction of the free radical cascades can be enhanced or even fortified with the inclusion of healing factors. Suitable healing factors for the compositions, methods and uses of the present disclosure include, but are not limited to:

Hyaluronic Acid (Hyaluronan or Hyaluronate)

Hyaluronic acid (hyaluronan or hyaluronate) is a non-sulfated glycosaminoglycan, distributed widely throughout connective, epithelial and neural tissues. It is one of the primary components of the extracellular matrix, and contributes significantly to cell proliferation and migration. Hyaluronan is a major component of the skin, where it is involved in tissue repair. While it is abundant in extracellular matrices, it contributes to tissue hydrodynamics, movement and proliferation of cells and participates in a wide number of cell surface receptor interactions, notably those including primary receptor CD44. The hyaluronidase enzymes degrade hyaluronan and there are at least seven types of hyaluronidase-like enzymes in humans, several of which are tumor suppressors. The degradation products of hyaluronic acid, the oligosaccharides and the very-low molecular weight hyaluronic acid, exhibit pro-angiogenic properties. In addition, recent studies show that hyaluronan fragments, but not the native high molecular mass of hyaluronan, can induce inflammatory responses in macrophages and dendritic cells in tissue injury. Hyaluronic acid is well suited to biological applications targeting the skin. Due to its high biocompatibility, it is used to stimulate tissue regeneration. Current studies evidenced hyaluronic acid appearing in the early stages of healing to physically create room for white blood cells that mediate the immune response. It is used in the synthesis of biological scaffolds for wound healing applications and in wrinkle treatment. In certain embodiments, the composition includes hyaluronic acid in the range of less than about 2% by weight of the total composition hyaluronic acid. In some embodiments, hyaluronic acid is present in an amount from about 0.001% to about 2%, or from about 0.002% to about 2%, or from about 0.002% to about 1% by weight of the total composition.

Glucosamine

Glucosamine is one of the most abundant monosaccharides in human tissues and a precursor in the biological synthesis of glycosylated proteins and lipids. It is commonly used in the treatment of osteoarthritis. The common form of glucosamine used is its sulfate salt. Glucosamine shows a number of effects including, anti-inflammatory activity, stimulation of the synthesis of proteoglycans and the synthesis of proteolytic enzymes. A suitable range of concentration over which glucosamine can be used in the present composition is from less than about 5% by weight of the total composition. In some embodiments, glucosamine is present in an amount from about 0.0001% to about 5%, or from about 0.0001% to about 3%, or from about 0.001% to about 3%, or from about 0.001% to about 1%, or about 0.01% to about 1%, or about 1% to about 3% by weight of the total composition.

Allantoin

Allantoin is a diureide of glyosilic acid. It has keratolytic effect, increases the water content of the extracellular matrix, enhances the desquamation of the upper layers of dead (apoptotic) skin cells, and promotes skin proliferation and wound healing. In certain embodiments, the composition includes in the range of less than about 1% by weight of the total composition allantoin. In some embodiments, allantoin is present in an amount from about 0.001% to about 1%, or from about 0.002% to about 1%, or from about 0.02% to about 1%, or from about 0.02% to about 0.5% by weight of the total composition.

Also, saffron can act as both a photon-transfer agent and a healing factor.

(g) Chelating Agents

Chelating agents can be included to promote smear layer removal in closed pockets and difficult to reach lesions. Chelating agents act as a metal ion quencher and as a buffer. Suitable chelating agents for the compositions, methods and uses of the disclosure include, but are not limited to:

Ethylenediaminotetraacetic Acid (EDTA)

Ethylenediaminotetraacetic acid (EDTA) is an amino acid and is used to sequester di- and trivalent metal ions. EDTA binds to metals via four carboxylate and two amine groups. EDTA forms especially strong complexes with Mn(III), Fe(III), Cu(III), Co(III). It is used to buffer solutions.

Ethylene Glycol Tetraacetic Acid (EGTA)

Ethylene glycol tetraacetic acid (EGTA) is related to EDTA, but with a much higher affinity for calcium than magnesium ions. It is useful for making buffer solutions that resemble the environment inside living cells.

(h) Additional Components

The compositions, methods, and uses of the disclosure can also include other ingredients such as humectants (e.g., glycerine, ethylene glycol, and propylene glycol), preservatives such as parabens, and pH adjusters such as sodium hydroxide, sodium bicarbonate, and HCl. In some embodiments, the pH of the composition is in or adjusted to the range of about 4 to about 10. In some embodiments, the pH of the composition is in or adjusted to the range of about 4 to about 9. In some embodiments, the pH of the composition is in or adjusted to the range of about 4 to about 8. In some embodiments, the pH of the composition is within the range of about 4 to about 7. In some embodiments, the pH of the composition is within the range of about 4 to about 6.5. In some embodiments, the pH of the composition is within the range of about 4 to about 6. In some embodiments, the pH of the composition is within the range of about 4 to about 5.5. In some embodiments, the pH of the composition is within the range of about 4 to about 5. In some embodiments, the pH of the composition is within the range of about 5.0 to about 8.0. In some embodiments, the pH of the composition is within the range of about 6.0 to about 8.0. In some embodiments, the pH of the composition is within the range of about 6.5 to about 7.5. In some embodiments, the pH of the composition is within the range of about 5.5 to about 7.5.

In some embodiments, the pH of the composition is in or adjusted to the range of 4 to 10. In some embodiments, the pH of the composition is in or adjusted to the range of 4 to 9. In some embodiments, the pH of the composition is in or adjusted to the range of 4 to 8. In some embodiments, the pH of the composition is within the range of 4 to 7. In some embodiments, the pH of the composition is within the range of 4 to 6.5. In some embodiments, the pH of the composition is within the range of 4 to 6. In some embodiments, the pH of the composition is within the range of 4 to 5.5. In some embodiments, the pH of the composition is within the range of 4 to 5. In some embodiments, the pH of the composition is within the range of 5.0 to 8.0. In some embodiments, the pH of the composition is within the range of 6.0 to 8.0. In some embodiments, the pH of the composition is within the range of 6.5 to 7.5. In some embodiments, the pH of the composition is within the range of 5.5 to 7.5.

In some embodiments, the compositions of the disclosure also include an aqueous substance (water) or an alcohol. Alcohols include, but are not limited to, ethanol, propanol, isopropanol, butanol, iso-butanol, t-butanol or pentanol. In some embodiments, the chromophore or combination of chromophores is in solution in a medium of the biophotonic composition. In some embodiments, the chromophore or combination of chromophores is in solution in a medium of the biophotonic composition, wherein the medium is an aqueous substance.

(3) Optical Properties of the Biophotonic Compositions

In certain embodiments, biophotonic compositions of the present disclosure are substantially transparent or translucent. The % transmittance of the biophotonic composition can be measured in the range of wavelengths from 250 nm to 800 nm using, for example, a Perkin-Elmer Lambda 9500 series UV-visible spectrophotometer. In some embodiments, transmittance within the visible range is measured and averaged. In some other embodiments, transmittance of the biophotonic composition is measured with the chromophore(s) omitted. As transmittance is dependent upon thickness, the thickness of each sample can be measured with calipers prior to loading in the spectrophotometer. Transmittance values can be normalized according to:

$${F}_{T - c o r r} ( λ , {t}_{2} ) = {[ {}^{- {σ}_{t}} ( λ ) {t}_{1} ]}^{\frac{{t}_{2}}{{t}_{1}}} = {[ {F}_{T - c o r r} ( λ , {t}_{1} ) ]}^{\frac{{t}_{2}}{{t}_{1}}} ,$$

where t₁=actual specimen thickness, t₂=thickness to which transmittance measurements can be normalized. In the art, transmittance measurements are usually normalized to 1 cm.

In certain embodiments, the biophotonic compositions are substantially opaque. In these embodiments, the biophotonic compositions may include light transmitting structures such as fibres, particles, networks, which are made of materials which can transmit light. The light transmitting structures can be waveguides such as optical fibres.

In some embodiments, the biophotonic composition has a transmittance that is more than about 20%, about 25%, about 30%, about 35%, about 40%, about 45%, about 50%, about 55%, about 60%, about 65%, about 70%, or about 75% within the visible range. In some embodiments, the transmittance exceeds 40%, 41%, 42%, 43%, 44%, or 45% within the visible range.

(4) Forms of the Biophotonic Compositions

The biophotonic compositions of the present disclosure may be a liquid, a gel, a cream, a paste, a putty, a semi-solid, or a solid. Biophotonic compositions in the liquid, gel, cream, paste or putty form can be applied by spreading, spraying, smearing, dabbing or rolling the composition on the target tissue. Biophotonic compositions of the putty, semi-solid or solid forms may be deformable. They may be elastic or non-elastic (i.e., flexible or rigid). The biophotonic compositions, for example, may be in a peel-off form (‘peelable’) to provide ease and speed of use. In certain embodiments, the tear strength and/or tensile strength of the peel-off form is greater than its adhesion strength. This may help handleability of the composition. It will be recognized by one of skill in the art that the properties of the peel-off biophotonic composition such as cohesiveness, flexibility, elasticity, tensile strength, and tearing strength, can be determined and/or adjusted by methods known in the art such as by selecting suitable thickening agents and adapting their relative ratios.

The biophotonic composition may be in a pre-formed shape. In certain embodiments, the pre-formed shape is in the form of, including, but not limited to, a film, a face mask, a patch, a dressing, or bandage. The biophotonic composition can be configured with a shape and/or size for application to a desired portion of a subject's body. For example, the biophotonic composition can be shaped and sized to correspond with a desired portion of the body to receive the biophotonic treatment. Such a desired portion of the body can be selected from, but not limited to, the group consisting of a skin, head, forehead, scalp, nose, cheeks, lips, ears, face, neck, shoulder, arm pit, arm, elbow, hand, finger, abdomen, chest, stomach, back, buttocks, sacrum, genitals, legs, knee, feet, toes, nails, hair, soft tissues, any boney prominences, and combinations thereof, and the like. The biophotonic composition may also be configured to be applied internally to a subject's body, such as on the luminal surface of a body cavity or organ of a subject, or be configured to be fitted or juxtapositioned to cover a substantial portion of the subject's external body surface or surface of a limb or other extremity. Thus, the biophotonic composition of the disclosure can be shaped and sized to be applied to any portion of tissue on a subject's body. For example, the biophotonic composition can be provided in the form of sock, hat, glove or mitten.

In certain aspects, the biophotonic composition forms part of a composite and can include fibres, particulates, non-biophotonic layers or biophotonic layers with the same or different compositions.

The biophotonic compositions of the present disclosure may have a thickness of, or be applied with a thickness of, from about 0.1 mm to about 50 mm, about 0.5 mm to about 20 mm, or about 1 mm to about 10 mm. It will be appreciated that the thickness of the biophotonic compositions will vary based on the intended use. In some embodiments, the biophotonic composition has a thickness of from about 0.1-1 mm. In some embodiments, the biophotonic composition has a thickness of about 0.5-1.5 mm, about 1-2 mm, about 1.5-2.5 mm about 2-3 mm, about 2.5-3.5 mm, about 3-4 mm, about 3.5-4.5 mm, about 4-5 mm, about 4.5-5.5 mm, about 5-6 mm, about 5.5-6.5 mm, about 6-7 mm, about 6.5-7.5 mm, about 7-8 mm, about 7.5-8.5 mm, about 8-9 mm, about 8.5-9.5 mm, about 9-10 mm, about 1.0-11 mm, about 11-12 mm, about 12-13 mm, about 13-14 mm, about 14-15 mm, about 1516 mm, about 16-17 mm, about 17-18 mm, about 18-19 mm, about 19-2.0 mm, about 20-72 mm, about 22-24 mm, about 24-26 mm, about 26-28 mm, about 28-30 mm, about 30-35 mm, about 35-40 mm, about 40-45 mm, or about 45-50 mm. In some embodiments, the biophotonic composition has a thickness of OA mm to 50 mm, 0.5 mm to 20 mm, or 1 mm to 10 mm. In some embodiments, the biophotonic composition has a thickness of from 0.1-1 mm. In some embodiments, the biophotonic composition has a thickness of 0.5-1.5 mm, 1-2 mm, 1.5-2.5 mm, 2-3 mm, 2.5-3.5 mm, 3-4 mm, 3.5-4.5 mm, 4-5 mm, 4.5-5.5 mm, 5-6 mm, 5.5-6.5 mm, 6-7 mm, 6.5-7.5 mm, 7-8 mm, 7.5-8.5 mm, 8-9 mm, 8.5-9.5 mm, 9-10 mm, 10-11 mm, 11-12 mm, 17-13 mm, 13-14 mm, 14-15 mm, 15-16 mm, 16-17 mm, 17-18 mm, 18-19 mm, 19-20 mm, 20-22 mm, 22-24 mm, 24-26 trim, 26-28 mm, 28-30 mm, 30-35 mm, 35-40 mm, 40-45 mm, or 45-50 mm.

(5) Biophotonic Composition and Photoactivatable Fiber/Fabric

The biophotonic composition of the present disclosure (e.g., Gel X as described herein) can be combined with a photoactivatable fiber to form an article of manufacture (e.g., a gel-mesh BioPhotonic System device) for use according to the methods disclosed herein. The article of manufacture comprises a biophotonic composition of the present disclosure, and a photoactivatable fiber having a plurality of a photoactivatable strand (or a filament). The photoactivatable strand (or filament) comprises a first thermoplastic polymer and a second thermoplastic polymer, and at least one photoactivatable agent. In some embodiments, the first polymer forms a core along the length of the strand, and the second polymer forms a sheath surrounding the core along the length of the strand. In some embodiments, the at least one photoactivable agent absorbs and emits light between about 400 nm and about 800 nm.

Suitable polymers that can be used according to the present disclosure are disclosed in publication WO 2016/065488 A1, incorporated by reference in its entirety. In some embodiments, the first polymer and the second polymer is a material selected from any one or more of acrylic, acrylonitrile butadiene styrene (ABS), polybenzimidazole (PBI), polycarbonate, polyether sulfone (PES), polyetherether ketone, (PEEK), polyetherimide (PE1), polyethylene (PE), polyphenylene oxide (PPO), polyphenylene sulfide (PPS), polypropylene (PP), polystyrene, polyvinyl chloride (PVC), teflon, polybutylene, polyethylene terephthalate (PET), polybutylene terephthalate (PBT), nylon, polylactic acid (PLA), polymethyl methacrylate polyester, polyurethane, rayon, and poly(methyl methacrylate) (PMMA). In some embodiments, the first polymer and the second polymer are the same material. In some embodiments, the first polymer and the second polymer are different materials. In some embodiments, the first and second polymer is nylon.

In some embodiments, the second polymer (sheath of the strand) and first polymer (core of the strand) are in an amount in any one of the following second polymer/first polymer ratios by weight: 5/95, 10/90, 15/85, 20/80, 25/75, 30/70, 35/65, 40/60, 45/55, 50/50, 55/45, 60/40, 65/35, 70/30, 75/25, 80/20, 85/15, or 90/10. In some embodiments, the second polymer (sheath of the strand) and first polymer (core of the strand) are in an amount in any one of the following second polymer/first polymer ratios by weight: 10/90, 25/75, 50/50, and 72/25.

In some embodiments, the first polymer that forms the core comprises at least one photoactivatable agent, as disclosed herein. For example, the photoactivable agent is selected from one or more of Eosin Y, Eosin B, Erythrosine, Fluorescein, or Rose Bengal. In some embodiments, the at least one photoactivatable agent(s) is present in the core polymer of a strand in an amount in the range of about 0.1% by weight, 0.2% by weight, 0.3% by weight, 0.4% by weight, 0.5% by weight, 0.6% by weight, 0.7% by weight, 0.8% by weight, 0.9% by weight, 1.0% by weight, 1.2% by weight, 1.4% by weight, 1.6% by weight, 1.8% by weight, 2.0% by weight, 2.2% by weight, 2.4% by weight, 2.6% by weight, 2.8% by weight, 3.0% by weight, 3.2% by weight, 3.4% by weight, 3.6% by weight, 3.8% by weight, 4.0% by weight, 4.2% by weight, 4.4% by weight, 4.6% by weight, 4.8% by weight, 5.0% by weight, 5.2% by weight, 5.4% by weight, 5.6% by weight, 5.8% by weight, 6.0% by weight, 6.2% by weight, 6.4% by weight, 6.6% by weight, 6.8% by weight, 7.0% by weight, 7.2% by weight, 7.4% by weight, 7.6% by weight, 7.8% by weight, 8.0% by weight, 8.2% by weight, 8.4% by weight, 8.6% by weight, 8.8% by weight, 9.0% by weight, 9.2% by weight, 9.4% by weight, 9.6% by weight, 9.8% by weight, 10.0% by weight, 10.5% by weight, 11.0% by weight, 11.5% by weight, 12.0% by weight, 12.5% by weight, 13.0% by weight, 13.5% by weight, 14.0% by weight, 14.5% by weight, 15.0% by weight, 15.5% by weight, 16.0% by weight, 16.5% by weight, 17.0% by weight, 17.5% by weight, 18% by weight, 18.5% by weight, 19.0% by weight, 19.5% by weight, 20.0% by weight, 21% by weight, 22% by weight, 23% by weight, 24% by weight, to about 25% by weight. In some embodiments, the at least one photoactivatable agent is present in the core polymer of a strand in an amount of about or at 1% by weight.

In some embodiments, each strand that forms the photoactivatable fiber disclosed herein possesses a non-uniform distribution of photoactivatable agent(s), thereby conferring unexpected advantages, as exemplified herein. For example, the photoactivatable agent(s) is distributed within just the core polymer of each strand, rendering the distribution of photoactivatable agent(s) non-homogeneous. Various methods of incorporating the at least one photoactivatable agent into the polymer are known in the art. In some embodiments, the at least one photoactivatable agent is incorporated into the polymer by compounding. Such a method is well-known in the art.

In some embodiments, the photoactivatable fiber comprises about 10 to about 360 strands (or filaments) described herein. In some embodiments, the photoactivatable fiber comprises about 10, 19, 64, or 360 strands described herein. In some embodiments, the photoactivatable fiber comprises about 19 strands described herein. In an exemplary embodiment, 19 strands described herein form a photoactivatable fiber.

In some embodiments, a plurality of a photoactivatable fiber form a fabric, or mesh, sometimes referred to herein as a sheath/core fiber mesh. As exemplified herein, a sheath/core fiber mesh is combined with a biophotonic composition disclosed herein to form an article of manufacture (e.g., a gel-mesh BioPhotonic System—BPS). Exemplary gel-mesh devices are described in detail in the Examples.

In a related embodiment, provided herein is a photoactivatable fiber having a plurality of a photoactivatable strand (or a filament). The photoactivatable strand (or filament) comprises a first thermoplastic polymer and a second thermoplastic polymer, and at least one photoactivatable agent. In some embodiments, the first polymer forms a core along the length of the strand, and the second polymer forms a sheath surrounding the core along the length of the strand. In some embodiments, the at least one photoactivable agent absorbs and emits light between about 400 nm and about 800 nm.

Suitable polymers that can be used to form the fibers described herein are disclosed in publication WO 2016/065488 A1, incorporated by reference in its entirety. In some embodiments, the first polymer and the second polymer is a material selected from any one or more of acrylic, acrylonitrile butadiene styrene (ABS), polybenzimidazole (PBI), polycarbonate, polyether sulfone (PES), polyetherether ketone, (PEEK), polyetherimide (PE1), polyethylene (PE), polyphenylene oxide (PPO), polyphenylene sulfide (PPS), polypropylene (PP), polystyrene, polyvinyl chloride (PVC), teflon, polybutylene, polyethylene terephthalate (PET), polybutylene terephthalate (PBT), nylon, polylactic acid (PLA), polymethyl methacrylate polyester, polyurethane, rayon, and poly(methyl methacrylate) (PMMA). In some embodiments, the first polymer and the second polymer are the same material. In some embodiments, the first polymer and the second polymer are different materials. In some embodiments, the first and second polymer is nylon.

In some embodiments, the second polymer (sheath of the strand) and first polymer (core of the strand) are in an amount in any one of the following second polymer/first polymer ratios by weight: 5/95, 10/90, 15/85, 20/80, 25/75, 30/70, 35/65, 40/60, 45/55, 50/50, 55/45, 60/40, 65/35, 70/30, 75/25, 80/20, 85/15, or 90/10. In some embodiments, the second polymer (sheath of the strand) and first polymer (core of the strand) are in an amount in any one of the following second polymer/first polymer ratios by weight: 10/90, 25/75, 50/50, and 72/25.

In some embodiments, the first polymer that forms the core comprises at least one photoactivatable agent, as disclosed herein. For example, the photoactivable agent is selected from one or more of Eosin Y, Eosin B, Erythrosine, Fluorescein, or Rose Bengal. In some embodiments, the at least one photoactivatable agent(s) is present in the core polymer of a strand in an amount in the range of about 0.1% by weight, 0.2% by weight, 0.3% by weight, 0.4% by weight, 0.5% by weight, 0.6% by weight, 0.7% by weight, 0.8% by weight, 0.9% by weight, 1.0% by weight, 1.2% by weight, 1.4% by weight, 1.6% by weight, 1.8% by weight, 2.0% by weight, 2.2% by weight, 2.4% by weight, 2.6% by weight, 2.8% by weight, 3.0% by weight, 3.2% by weight, 3.4% by weight, 3.6% by weight, 3.8% by weight, 4.0% by weight, 4.2% by weight, 4.4% by weight, 4.6% by weight, 4.8% by weight, 5.0% by weight, 5.2% by weight, 5.4% by weight, 5.6% by weight, 5.8% by weight, 6.0% by weight, 6.2% by weight, 6.4% by weight, 6.6% by weight, 6.8% by weight, 7.0% by weight, 7.2% by weight, 7.4% by weight, 7.6% by weight, 7.8% by weight, 8.0% by weight, 8.2% by weight, 8.4% by weight, 8.6% by weight, 8.8% by weight, 9.0% by weight, 9.2% by weight, 9.4% by weight, 9.6% by weight, 9.8% by weight, 10.0% by weight, 10.5% by weight, 11.0% by weight, 11.5% by weight, 12.0% by weight, 12.5% by weight, 13.0% by weight, 13.5% by weight, 14.0% by weight, 14.5% by weight, 15.0% by weight, 15.5% by weight, 16.0% by weight, 16.5% by weight, 17.0% by weight, 17.5% by weight, 18% by weight, 18.5% by weight, 19.0% by weight, 19.5% by weight, 20.0% by weight, 21% by weight, 22% by weight, 23% by weight, 24% by weight, to about 25% by weight. In some embodiments, the photoactivatable agent is present in the core polymer of a strand in an amount of about or at 1% by weight.

In some embodiments, each strand that forms the photoactivatable fiber disclosed herein possesses a non-uniform distribution of photoactivatable agent(s), thereby conferring unexpected advantages, as exemplified herein. For example, the photoactivatable agent(s) is distributed within just the core polymer of each strand, rendering the distribution of photoactivatable agent(s) non-homogeneous. Various methods of incorporating the at least one photoactivatable agent into the polymer are known in the art. In some embodiments, the at least one photoactivatable agent is incorporated into the polymer by compounding. Such a method is well-known in the art.

In some embodiments, the photoactivatable fiber comprises about 10 to about 360 strands (or filaments) described herein. In some embodiments, the photoactivatable fiber comprises about 10, 19, 64, or 360 strands described herein. In some embodiments, the photoactivatable fiber comprises about 19 strands described herein. In an exemplary embodiment, 19 strands described herein form a photoactivatable fiber.

In some embodiments, disclosed herein is a photoactivatable fabric (or mesh) comprising a plurality of a photoactivatable fiber as described herein.

In some embodiments, the photoactivatable fabric (or mesh) described herein minimizes leaching of the chromophore to, e.g., below detection limit, as described herein and/or maximizes fluorescence of the chromophore.

(6) Methods of Use

The biophotonic compositions of the present disclosure (alone or as part of a mesh device as disclosed herein) may have medical benefits. They can be used for treating rare diseases that afflict skin or soft tissues. In some embodiments, the rare disease that afflicts skin or soft tissues include, but are not limited to, CHILD syndrome and in particular the ichthyosiform erythroderma aspect of CHILD syndrome; dermatomyositis; hidradenitis suppurativa; acquired ichthyosis as well as hereditary ichthyosis; lichen myxedematosus and scleromyxedema; pemphigus; porphyria disorders; epidermolysis bullosa; Ehlers-Danlos syndrome; cutis hyperelastica; eosinophilic fasciitis; osteogenesis imperfect; Winchester syndrome; Hailey-Hailey disease (also referred to as Benign Chronic Familial Pemphigus); and scleroderma.

Accordingly, in certain embodiments, the present disclosure provides a method for treating a rare disease that afflicts skin or soft tissues, the method comprising: applying a biophotonic composition of the present disclosure to the tissue in need of treatment, and illuminating the biophotonic composition with light having a wavelength that overlaps with an absorption spectrum of the at least one fungal-derived chromophore (i.e., first chromophore) present in the biophotonic composition. In some embodiments, where a second chromophore is included in the biophotonic composition, the composition is illuminated with light having a wavelength that is absorbed by the at least second chromophore. In some embodiments, the rare disease is selected from CHILD syndrome and in particular the ichthyosiform erythroderma aspect of CHILD syndrome; dermatomyositis; hidradenitis suppurativa; acquired ichthyosis as well as hereditary ichthyosis; lichen myxedematosus and scleromyxedema; pemphigus; porphyria disorders; epidermolysis bullosa; Ehlers-Danlos syndrome; cutis hyperelastica; eosinophilic fasciitis; osteogenesis imperfect; Winchester syndrome; Hailey-Hailey disease (also referred to as Benign Chronic Familial Pemphigus); and scleroderma.

In the methods of the present disclosure, any source of actinic light can be used. The source of actinic light may be a natural source, such as sunlight, or may be a generated source. Any type of halogen, LED or plasma arc lamp, or laser may be suitable source of generated actinic light. The primary characteristic of suitable sources of actinic light will be that they emit light in a wavelength (or wavelengths) appropriate for activating the one or more photoactivators present in the composition. The appropriate wavelength (or wavelengths) may be in the visible range of wavelengths of light, or may be of a shorter wavelength or of a longer wavelength (e.g. infra red) than visible light. In some embodiments, an argon laser is used. In other embodiments, a potassium-titanyl phosphate (KTP) laser (e.g. a GreenLight™ laser) is used. In yet other embodiments, a LED lamp such as a photocuring device is the source of the actinic light. In yet other embodiments, the source of the actinic light is a source of light having a wavelength between about 200 to about 800 nm. In other embodiments, the source of the actinic light is a source of visible light having a wavelength between about 400 and about 600 nm. In other embodiments, the source of the actinic light is a source of visible light having a wavelength between about 400 and about 700 nm or about 400 nm to about 750 nm. In yet other embodiments, the source of the actinic light is blue light. In yet other embodiments, the source of the actinic light is red light. In yet other embodiments, the source of the actinic light is green light. In some embodiments, the LED lamp may comprise LEDs of more than one wavelength, for example, LEDs that emit at a blue light range and other LEDs that emit at the green light or yellow light range or other ranges of light. Furthermore, the source of actinic light should have a suitable power density. Suitable power density for non-collimated light sources (LED, halogen or plasma lamps) are in the range from about 0.1 mW/cm² to about 200 mW/cm², or about 30 mW/cm² to about 150 mW/cm². Suitable power density for laser light sources are in the range from about 0.5 mW/cm² to about 0.8 mW/cm².

In some embodiments of the methods of the present disclosure, the light has an energy at the subject's skin surface of between about 0.1 mW/cm² and about 500 mW/cm², or 0.1-300 mW/cm², or 0.1-200 mW/cm², wherein the energy applied depends at least on the condition being treated, the wavelength of the light, the distance of the skin from the light source and the thickness of the biophotonic composition. In certain embodiments, the light at the subject's skin is between about 1 mW/cm²-40 mW/cm², or about 20 mW/cm²-60 mW/cm², or about 40 mW/cm²-80 mW/cm², or about 60 mW/cm²-100 mW/cm², or about 80 mW/cm²-120 mW/cm², or about 100 mW/cm²-140 mW/cm², or about 30 mW/cm²-180 mW/cm², or about 120 mW/cm²-160 mW/cm², or about 140 mW/cm²-180 mW/cm², or about 160 mW/cm²-200 mW/cm², or about 110 mW/cm²-240 mW/cm², or about 110 mW/cm²-150 mW/cm², or about 190 mW/cm²-240 mW/cm².

The activation of the chromophore(s) within the biophotonic compositions of the disclosure may take place almost immediately on illumination (femto- or pico seconds). A prolonged exposure period may be beneficial to exploit the synergistic effects of the absorbed, reflected and reemitted light of the biophotonic compositions of the present disclosure and its interaction with the tissue being treated. In some embodiments, the time of exposure to actinic light of the tissue or skin or biophotonic composition is a period between 1 minute and 5 minutes. In other embodiments, the time of exposure to actinic light of the tissue or skin or biophotonic composition is a period between 1 minute and 5 minutes. In some other embodiments, the biophotonic composition is illuminated for a period between 1 minute and 3 minutes. In certain embodiments, light is applied for a period of about 1-30 seconds, about 15-45 seconds, about 30-60 seconds, about 0.75-1.5 minutes, about 1-2 minutes, about 1.5-2.5 minutes, about 2-3 minutes, about 2.5-3.5 minutes, about 3-4 minutes, about 3.5-4.5 minutes, about 4-5 minutes, about 5-10 minutes, about 5-9 minutes, about 5-8 minutes, about 10-15 minutes, about 15-20 minutes, about 20-25 minutes, or about 20-30 minutes. In some embodiments, light is applied for a period of 1 second. In some embodiments, light is applied for a period of 5 seconds. In some embodiments, light is applied for a period of 10 seconds. In some embodiments, light is applied for a period of 20 seconds. In some embodiments, light is applied for a period of 30 seconds. In some embodiments, the biophotonic composition is illuminated for a period less than 30 minutes. In some embodiments, the biophotonic composition is illuminated for a period less than 20 minutes. In some embodiments, the biophotonic composition is illuminated for a period less than 15 minutes. In some embodiments, the biophotonic composition is illuminated for a period less than 10 minutes. In some embodiments, the biophotonic composition is illuminated for a period less than 5 minutes. In some embodiments, the biophotonic composition is illuminated for a period less than 1 minute. In some embodiments, the biophotonic composition is illuminated for a period less than 30 seconds. In some embodiments, the biophotonic composition is illuminated for a period less than 20 seconds. In some embodiments, the biophotonic composition is illuminated for a period less than 10 seconds. In some embodiments, the biophotonic composition is illuminated for a period less than 5 seconds. In some embodiments, the biophotonic composition is illuminated for a period less than 1 second. The treatment time may range up to about 90 minutes, about 80 minutes, about 70 minutes, about 60 minutes, about 50 minutes, about 40 minutes, about 30 minutes or about 20 minutes. It will be appreciated that the treatment time can be adjusted in order to maintain a dosage by adjusting the rate of fluence delivered to a treatment area. For example, the delivered fluence may be about 4 J/cm² to about 60 J/cm², about 10 J/cm² to about 60 J/cm², about 10 J/cm² to about 50 J/cm², about 10 J/cm² to about 40 J/cm², about 10 J/cm² to about 30 J/cm², about 20 J/cm² to about 40 J/cm², about 15 J/cm² to 25 J/cm², or about 10 J/cm² to about 20 J/cm². The delivery fluence may also be adjusted in terms of levels of singlet oxygen released.

In certain embodiments, the biophotonic compositions of the disclosure may be re-illuminated at certain intervals, such as 1, 2, 3, 4, 5, 6, 7, 8, 9, 10, 11, 12, 24, 36, or 48 hours. In yet other embodiments, the source of actinic light is in continuous motion over the treated area for the appropriate time of exposure. In yet other embodiments, the biophotonic compositions of the disclosure may be illuminated until the biophotonic composition is at least partially photobleached or fully photobleached.

In some embodiments, the chromophore(s) in the biophotonic compositions of the disclosure can be photoexcited by ambient light including from the sun and overhead lighting. In some embodiments, the chromophore(s) can be photoactivated by light in the visible range of the electromagnetic spectrum. The light can be emitted by any light source such as sunlight, light bulb, an LED device, electronic display screens such as on a television, computer, telephone, mobile device, flashlights on mobile devices. In the methods of the present disclosure, any source of light can be used. For example, a combination of ambient light and direct sunlight or direct artificial light may be used. Ambient light can include overhead lighting such as LED bulbs, fluorescent bulbs, and indirect sunlight.

In the methods and uses of the present disclosure, the biophotonic compositions may be removed from the skin following application of light. In some embodiments, the biophotonic composition is peeled off, or is washed off, the tissue being treated after a treatment time. In other embodiments, the biophotonic composition is left on the tissue for an extended period of time and re-activated with direct or ambient light at appropriate times to treat the condition.

In certain embodiments of the methods and uses of the present disclosure, the biophotonic compositions can be applied to the tissue, such as on the face or afflicted site, once, twice, three times, four times, five times or six times a week, daily, or at any other frequency. The total treatment time can be one week, two weeks, three weeks, four weeks, five weeks, six weeks, seven weeks, eight weeks, nine weeks, ten weeks, eleven weeks, twelve weeks, or any other length of time deemed appropriate. In certain embodiments, the total tissue area to be treated may be split into separate areas (cheeks, forehead), and each area treated separately. For example, the composition may be applied topically to a first portion, and that portion illuminated with light, and the biophotonic composition then removed. Then the composition is applied to a second portion, illuminated and removed. Finally, the composition is applied to a third portion, illuminated and removed.

In certain embodiments, the biophotonic compositions of the disclosure can be used to treat rare diseases that afflict skin or soft tissues. Rare diseases that afflict skin or soft tissues for which the compositions may be used to treat or alleviate one or more symptoms thereof may include, but are not limited to, CHILD syndrome and in particular the ichthyosiform erythroderma aspect of CHILD syndrome; dermatomyositis; hidradenitis suppurativa; acquired ichthyosis as well as hereditary ichthyosis; lichen myxedematosus and scleromyxedema; pemphigus; porphyria disorders; epidermolysis bullosa; Ehlers-Danlos syndrome; cutis hyperelastica; eosinophilic fasciitis; osteogenesis imperfect; Winchester syndrome; Hailey-Hailey disease (also referred to as Benign Chronic Familial Pemphigus); and scleroderma. In this case, the biophotonic compositions may be applied at regular intervals such as once a week, or at an interval deemed appropriate by the physician or any other health care provider.

In the methods and uses of the present disclosure, additional components may optionally be included in the biophotonic compositions or used in combination with the biophotonic compositions. Such additional components include, but are not limited to, healing factors, antimicrobials, oxygen-rich agents, wrinkle fillers such as botox, hyaluronic acid and polylactic acid, anti-fungal, anti-bacterial, anti-viral agents and/or agents that promote collagen synthesis. These additional components may be applied to the skin in a topical fashion, prior to, at the same time of, and/or after topical application of the biophotonic compositions of the present disclosure. Suitable healing factors comprise compounds that promote or enhance the healing or regenerative process of the tissues on the application site. During the photoactivation of a biophotonic composition of the present disclosure, there may be an increase of the absorption of molecules of such additional components at the treatment site by the skin or the mucosa. In certain embodiments, an augmentation in the blood flow at the site of treatment can observed for a period of time. An increase in the lymphatic drainage and a possible change in the osmotic equilibrium due to the dynamic interaction of the free radical cascades can be enhanced or even fortified with the inclusion of healing factors. Healing factors may also modulate the biophotonic output from the biophotonic composition such as photobleaching time and profile, or modulate leaching of certain ingredients within the composition. Suitable healing factors include, but are not limited to glucosamines, allantoin, saffron, agents that promote collagen synthesis, anti-fungal, anti-bacterial, anti-viral agents and wound healing factors such as growth factors.

Rare Diseases of Skin and Soft Tissues

The biophotonic compositions, methods, and uses of the disclosure are useful in the treatment of rare diseases that afflict skin or soft tissues. Rare diseases that afflict skin or soft tissues for which the compositions may be used to treat or alleviate one or more symptoms thereof may include, but are not limited to, CHILD syndrome and in particular the ichthyosiform erythroderma aspect of CHILD syndrome; dermatomyositis; hidradenitis suppurativa; acquired ichthyosis as well as hereditary ichthyosis; lichen myxedematosus and scleromyxedema; pemphigus; porphyria disorders; epidermolysis bullosa; Ehlers-Danlos syndrome; cutis hyperelastica; eosinophilic fasciitis; osteogenesis imperfect; Winchester syndrome; Hailey-Hailey disease (also referred to as Benign Chronic Familial Pemphigus); and scleroderma. Such rare diseases may be manifested by collagen production and/or deposition abnormality. Other rare diseases of the skin or soft tissues for which the present compositions may be used to treat or alleviate one or more symptoms thereof may also be found by reference to Touitou et al. (2013) “The expanding spectrum of rare monogenic autoinflammatory diseases” Orphan Journal of Rare Diseases, volume 8, pages 162-174.

Hailey-Hailey disease (HHD) is a rare skin disease that has a genetic origin (autosomal dominant) that is caused by a defect in keratinocyte adhesion due to the ATP2 C1 gene mutation. This gene codes for the protein SPCA1 (Secretory Pathway Calcium/manganese-ATPase), a calcium and manganese pump. Clinically, HHD is characterized by abnormal keratinocyte adhesion and proliferation (hyperkeratosis), while histologically, it is characterized by disruption of cell-to-cell adhesion (acantholysis=desmosome decomposition) in the suprabasal layer of the epidermis with formation of intraepidermal bullae. The exact prevalence of HHD, which males and females are equally affected, is not known, however, it is estimated to occur at a prevalence of 1/50,000 (6400 patients) in the USA although this estimated rate remains to be validated. HDD lesions generally begin 20 and 40 years of age, and appear as vesicular or erosive lesions, blistering skin rash and hyperkeratosis predominantly on the intertriginous areas with intact blisters being rare. HDD are typically found to be painful by patients suffering from this rare disease. Although in a substantial number of patients afflicted with HDD the disease is manifested as a mild condition (and such patients are able to lead normal lives, with their condition only being a nuisance), in the more severely affect patients, these patients suffer from more persistent painful raw areas of the skin with development of superficial blisters. There is no cure for HHD, and treatments that have been used in the art have focused at reducing symptoms, such treatments including:
Cool compresses, dressings, mild corticosteroid creams as such have been effective in treating mild lesions and preventing exacerbations
Combinations of corticosteroid/antibiotic creams are helpful to alleviate associated infections
For most serious cases of HDD, systemic antibiotics and stronger corticosteroid creams have been utilized
Calcipotriol (Daivonex) cream has been shown to be useful in some patients
Different oral medications (retinoids, cyclosporine, dapsone, methotrexate) have been reported in single cases as being partially effective
Use of Botulinum toxin A shown benefits in refractory disease
UVB-therapy and PDT successfully used in different case series

For cases of severe HDD that are not responsive to any topical or systemic therapy, destructive therapy (e.g. CO₂ laser, dermabrasion) or surgical excision with grafting has been considered as a treatment option, however, the effectiveness of such destructive therapies in treating HDD has not been demonstrated. Another treatment option that has been considered in the art is that of Afamelanotide (Clinuvel Pharmaceuticals Ltd., Australia), which is a linear peptide which activates eumelanin of the skin and which is administered underneath the skin as a dissolvable implant.

Regarding epidermolysis bullosa (EB), this rare disease is usually inherited, and inherited EB has been shown to result from mutations of structural proteins, with the most common mutations are on genes coding for keratin 5 and keratin 14, and for all types of dystrophic EB result being formed due to mutations within the type VII collagen. Inherited EB is transmitted as either an autosomal dominant or autosomal recessive disease, depending on EB type and subtype. In general, the severity of skin and extracutaneous disease is a reflection of the type of mutation which is present, as well as the ultrastructural location of the targeted protein. EB is estimated to have a prevalence of 1 out of every 20,000 live births in the United States, and it is estimated to afflict 300,000 to 400,000 people worldwide. There are four major types of EB: (1) EB simplex (EBS), which affects the epidermis, (2) Junctional EB (JEB), which affects the basement membrane, (3) Dystrophic EB (DEB), which affects the dermis, and (4) Kindler syndrome, which affects multiple levels (basement membrane and dermis). EBS is the most common EB sub-type, with the main clinical signs being blistering in the epidermis. The blisters usually do not result in scars with this mild type, which mainly the soles of the feet and the palms. The age at which EBS first appears usually begins at birth or in early infancy, and this sub-type is has an autosomal dominant gene expression pattern, with the mutation being in the genes encoding for keratins 5 and 14. The JEB sub-type, which usually begins at birth, is a severe form of the disease, with the clinical signs: tissue separation and blistering in the deeper layer of skin. JEB may affect entire body, and a baby with this condition may develop a hoarse-sounding cry from continual blistering and scarring of the vocal cords. JEB is caused by an autosomal recessive mutation of genes encoding for keratin. The DEB sub-type of EB may be manifested as a mild to severe form with the degree of dystrophy being associated with the skin symptoms. The age of first appearance: generally becomes apparent at birth or during early childhood, and this sub-type may be inherited as a either dominant or recessive mutation of a gene helping to produce type VII collagen. The Kindler syndrome sub-type of EB is a very rare, recessive form of EB, and, clinically, patients afflicted with this EB sub-type have blisters that appear across the skin layers. It is not uncommon for patients afflicted with Kindler syndrome to show improvement in their condition over time, even to the point where the malady is no longer manifested. Kindler syndrome is the only EB sub-type that causes hypopigmentation of skin when exposed to the sun, and the disease typically is apparent at birth or soon thereafter.

Most EB patients, particularly those with EBS and Dominant DEB, have normal life expectancies, but they suffer from significant morbidity. In contrast, patients with JEB are at major risk of death during the first few years of life, and patients with Recessive DEB, particularly those with severe generalized form, are at risk of death on or after young adulthood from metastatic squamous cell carcinoma.

With respect to treating patients with EB, prevention of blistering though application of skin moisturization, soft clothing, moderate temperature exposure, and adhesive bandages have proven useful in the art. Patients, however, face a requirement of careful wound care management with regular dressing changes: frequent bandaging is in order to keep blisters clean and protected. Type of dressing depends on the severity of the lesion and the type of EB. Generally, it has been recommended in the art to use sterile synthetic non-adhesive dressings such as hydro-colloids for EB patients, along with use of local or systemic antibiotics, if required, in order to control the bacteria burden. Other aspects of treating patients afflicted with EB include, but may not be limited to, pain management, management of pruritus (which may be intense in dystrophic EB), bathing (with added salts), nutritional support (in order to provide good nutrition, along with supplementation in iron, calcium and vitamin D), psychological support, and symptomatic treatments as needed, such as dental and ophthalmologic examinations. Other treatment modalities have also been utilized for treating EB patients, including surgery (skin grafting, dilation of the esophagus, repair of hands deformities, and removal of any squamous cell carcinoma), along with ex-vivo gene replacement, transplantation of allogeneic fibroblasts, transplantation of bone marrow-derived stem cells, and a promotion of wound healing through application of various pharmaceutical products, such as SD-101 (Zorbliza™-Scioderm, Inc., Durham, N.C.), Diacerein (TWi Biotechnology, Taipei, Taiwan) or by infusion of recombinant proteins (type VII collagen for DEB (Fibrocell, Exton, Pa.)).

Regarding hidradenitis suppurativa (HS), which is also referred to as Verneuil's disease or acne inversa, this is a chronic, inflammatory, recurrent, debilitating skin disease of the hair follicle with painful, deep-seated, inflamed lesions in the apocrine gland-bearing areas. The most common areas of the body that become affected are the axillae, inguinal and anogenital regions. The disease primarily affects women versus men, with the women/men affected ratio of 3:1. HS usually develops after puberty with a peak age of onset in the early twenties, and a peak of severity after a mean disease duration of 6.4 years and thereafter the severity gradually decreases over time. Although the exact prevalence of HS remains unknown because of the difficulty in collecting and extrapolating data, it is estimated that in the U.S., the prevalence is roughly 0.05%. The physiopathology of HS is mostly unknown; the disease is considered to likely be multifactorial in its nature, including having a genetic component, an infectious components, and hormonal and immunologic factors. At least 30% of patients may have someone else in their family diagnosed with HS, which indicates an autosomal dominant inheritance pattern. From an etiological perspective, it is believed that the primary event is a hyperkeratinization of the follicular infundibulum, which is followed by follicular occlusion, dilatation and rupture, and then due to a spread of bacterial and cellular remnants, there is a triggering of the local inflammatory response, and a wide range of bacteria that may become associated with HS: Staph. aureus, Strep. agalactiae, coagulase-negative Staphylococci, milleri group Streptococci, anaerobes and Corynebacteria.

HS patients will become afflicted with recurrent painful, deep inflammatory nodules, and chronic, draining sinus tracts. HS is extremely heterogeneous in terms of severity and comorbidities, and HS has the highest impact on patients' Quality of Life (QoL) among dermatological diseases; there is a high comorbidity associated with HS, and the severity of HS appears to be more debilitating in many aspects of life than even psoriasis. The HS-associated co-morbidities may include one or more of obesity, pyoderma, arthritis, Crohn's disease, anaemia and lymphedema. As well, there are a number of complications that HS patients suffer from, including acute skin infections, lymphatic obstruction/lymphedema, long-standing inflammation of the genitoanal area, squamous cell carcinoma, rheumatological disorders, depression, poor social integration, and other metabolic syndromes (such as hypertriglyceridemia and hyperglycemia). There is no known cure for HS, and treatments known in the art for HS have only aimed at reducing the severity of the symptoms; these include customized bandages, psychosocial support measures, pain control, topical therapy, surgery and immunosuppressive drugs, and laser therapy.

Regarding scleroderma, this is a rare autoimmune connective tissue disorder that is characterized by abnormal hardening of the skin, and that may also affect other organs.

Scleroderma is frequently linked with rheumatology disorders. There are two main types of scleroderma: Localized scleroderma and Systemic sclerosis, with the prevalence in the USA of each being: localized scleroderma (=cutaneous form): estimations of 1-9/100,000 (between 3,200 to 28,800 patients), and systemic sclerosis: 1/6,500 (approximately 50,000 patients). Women are predominantly affected over men (F/M ratio=4:1), and sceleroderma may affect all groups of ages, although systemic sclerosis appears generally in patients who are between 30-40 years of age.

The exact cause of scleroderma is unknown, although there is a localized overproduction of collagen due to an autoimmune reaction, and vascular disorders that may be linked to TGF-beta and PDGF, while the diminution of lesional cutaneous blood vessels may be attributed to antiendothelial cell autoantibodies. Scleroderma may involve genetic and infectious diseases, with a possible link with exposure to certain chemicals.

Localized scleroderma is characterized by a skin fibrosis and a vascular dysfunction in the skin. Clinically, the disease manifests as a presence of cutaneous plaques (morphea) or strips (linear scleroderma) that often occurs on arms, legs and forehead. Linear scleroderma is more common in children and adolescents, and the malady may be associated with rheumatologic manifestations: joint pain (Arthralgia). Patients with localized scleroderma experience a raft of symptoms the skin is expected, but the skin discoloration may last for many years and could remain permanent. Linear scleroderma remains active for two to five years, but can last longer in some cases. Sometimes patients develop recurrences after a period of what was thought to be inactive disease. Patients with localized scleroderma rarely progress to systemic sclerosis.

For patients' afflicted with systemic sclerosis, they may exhibit a multiplicity of symptoms, which may very depending on the body structure affected. These patients' may be afflicted with vascular obliteration and fibrosis not only in the skin but also in organs. T, signs and symptoms of systemic scleroderma usually begin with episodes of Raynaud phenomenon, which can occur weeks to years before fibrosis (fingers and toes of affected individuals turn white or blue in response to cold temperature or other stresses). There is generally, a thickening and hardening of the skin due to fibrosis, first in fingers (sclerodactyly) and the possibility of ulcers on fingers, calcinosis, and telangiectasia, with the main organs affected: lungs, heart, digestive tract, kidneys. Although scleroderma treatment options have been tried, there remain no consensus on the treatment, and only symptomatic treatments are available in order to improve quality of life.

(7) Kits

The present disclosure also provides kits containing the biophotonic compositions and/or providing any of the components required for preparing biophotonic compositions of the present disclosure.

In some embodiments, the kit includes a biophotonic composition of the present disclosure. In some embodiments, the kit includes containers comprising the components that can be used to make the biophotonic composition of the present disclosure. The different components making up the biophotonic compositions of the present disclosure may be provided in separate containers. For example, in embodiments where the biophotonic composition comprises a peroxide source, the peroxide or peroxide precursor of the biophotonic composition may be provided in a container separate from the chromophore(s). Examples of such containers are dual chamber syringes, dual chamber containers with removable partitions, sachets with pouches, and multiple-compartment blister packs. Another example is one of the components being provided in a syringe which can be injected into a container of another component.

In some embodiments, the kit includes a photoactivatable fabric (or mesh) comprising a plurality of a photoactivatable fiber as described herein. In some embodiments, the kit includes an article of manufacture as disclosed herein (e.g., that comprises a biophotonic composition of the present disclosure, and a photoactivatable fiber having a plurality of a photoactivatable strand or filament).

In other embodiments, the kit comprises a systemic drug for augmenting the treatment of the biophotonic composition of the present disclosure. For example, the kit may include a systemic or topical antibiotic, hormone treatment, or a negative pressure device. The kit may also include instructions for use. The carrier medium may be included together with any of the other three components. In some embodiments, the kit comprises a means for applying the components of the biophotonic compositions such as a spatula, a syringe, or the like.

In certain aspects, there is provided a container comprising a chamber for holding a biophotonic composition, and an outlet in communication with the chamber for discharging the biophotonic composition from the container, wherein the biophotonic composition comprises at least one chromophore in a carrier medium which can form a biophotonic composition after being discharged from the sealed chamber, for example on contact with skin or on illumination with a light. In certain embodiments, the chamber is partitioned such that the chromophore(s), and the peroxide or peroxide precursor are kept in separate compartments until discharged from the container or during discharging from the container.

In certain embodiments, the kit comprises a dressing or a mask. The dressing or mask may be a porous or semi-porous structure for receiving the biophotonic composition. The dressing or mask may also comprise woven or non-woven fibrous materials. The biophotonic composition or its precursor can be incorporated, such as by injection, into the dressing.

In certain embodiments of the kit, the kit may further comprise a light source such as a portable light with a wavelength appropriate to activate the chromophore(s) of the biophotonic composition. The portable light may be battery operated or re-chargeable. The light source may comprise LEDs.

Written instructions on how to use the biophotonic compositions in accordance with the present disclosure may be included in the kit, or may be included on or associated with the containers comprising the compositions or components making up the biophotonic compositions of the present disclosure. The instructions can include information on how to form the biophotonic composition from the individual components or biophotonic composition precursors provided with the kit.

Identification of equivalent biophotonic compositions, methods and kits are well within the skill of the ordinary practitioner and would require no more than routine experimentation, in light of the teachings of the present disclosure.

Variations and modifications will occur to those of skill in the art after reviewing this disclosure. The disclosed features may be implemented, in any combination and subcombinations (including multiple dependent combinations and subcombinations), with one or more other features described herein. The various features described or illustrated above, including any components thereof, may be combined or integrated in other systems. Moreover, certain features may be omitted or not implemented. Examples of changes, substitutions, and alterations are ascertainable by one skilled in the art and could be made without departing from the scope of the information disclosed herein.

### EXAMPLES

The examples below are given so as to illustrate the practice of various embodiments of the present disclosure. They are not intended to limit or define the entire scope of this disclosure.

### Example 1: Brief Description of the Biophotonic Compositions

Blank gel: A gel comprising water and other components, but lacking a chromophore and urea peroxide.

Gel A: A gel comprising water, a carbomer, and urea peroxide and a combination of Maitake extract (aqueous, a Grifola species), Reishi extract (aqueous, a Ganoderma species), Agarikon extract (aqueous, a Laricifomes species), Cordyceps extract (aqueous), Rose Bengal and Eosin Y.

Gel B: A gel comprising water, a carbomer, and urea peroxide and a combination of Maitake extract (aqueous, a Grifola species), Reishi extract (aqueous, a Ganoderma species), Agarikon extract (aqueous, a Laricifomes species), Cordyceps extract (aqueous), and Rose Bengal.

Gel C: A gel comprising water, a carbomer, and urea peroxide and a combination of Maitake extract (aqueous, a Grifola species), Reishi extract (aqueous, a Ganoderma species), Agarikon extract (aqueous, a Laricifomes species), Agaricus extract (aqueous), Matsutake extract (aqueous, a Tricholoma species), and Eosin Y.

Gel D: A gel comprising water, a carbomer, and urea peroxide and a combination of Maitake extract (aqueous, a Grifola species), Reishi extract (aqueous, a Ganoderma species), Agarikon extract (aqueous, a Laricifomes species), and Cordyceps extract (aqueous).

Gel E: A gel comprising water, a carbomer, and urea peroxide and a combination of Maitake extract (aqueous, a Grifola species), Reishi extract (aqueous, a Ganoderma species), Agarikon extract (aqueous, a Laricifomes species), Agaricus extract (aqueous), Matsutake extract (aqueous, a Tricholoma species), and Rose Bengal.

Gel F: A gel comprising water, a carbomer, and urea peroxide and a combination of Maitake extract (aqueous, a Grifola species), Reishi extract (aqueous, a Ganoderma species), Agarikon extract (aqueous, a Laricifomes species), Cordyceps extract (aqueous), and Eosin Y.

Gel G: A gel comprising water, a carbomer, and urea peroxide; and a combination of Maitake extract (aqueous, a Grifola species), Reishi extract (aqueous, a Ganoderma species), Shiitake extract (aqueous, a Lentinula species), and Rose Bengal.

Gel H: A gel comprising water, a carbomer, and urea peroxide and a combination of Maitake extract (aqueous, a Grifola species), Reishi extract (aqueous, a Ganoderma species), Shiitake extract (aqueous, a Lentinula species), and Eosin Y.

Gel I: A gel comprising water, a carbomer, and urea peroxide and a combination of Maitake extract (aqueous, a Grifola species), Reishi extract (aqueous, a Ganoderma species), Agarikon extract (aqueous, a Laricifomes species), Agaricus extract (aqueous), and Matsutake extract (aqueous, a Tricholoma species).

Gel J: A gel comprising water, a carbomer, and urea peroxide and a combination of Maitake extract (aqueous, a Grifola species), Reishi extract (aqueous, a Ganoderma species), Agarikon extract (aqueous, a Laricifomes species), Agaricus extract (aqueous), Matsutake extract (aqueous, a Tricholoma species), Rose Bengal and Eosin Y.

### Example 2: Preparation of the Biophotonic Compositions

For the preparation of the biophotonic compositions of the disclosure, the following methodology was utilized. Fungi were sourced from commercial retailers, either as whole, raw mushrooms or in a processed, capsule format. If purchased in the raw form, an aliquot of the purchased, raw mushroom was, firstly, subjected to a size-reduction treatment by crushing the sample into a semi-fine, homogeneous powder using a blender device. Thereafter, for both the ground mushroom preparation and the capsular format preparation, 5 grams of the particular mushroom were then added to 25 mL of propylene glycol and the resulting solution was continuously stirred at low speed over the course of a 15 day period. At the completion of the stirring period, the resulting product is left unfiltered with the particulate (solid) fraction remaining on the bottom of the mixing container while samples are withdrawn from the overlying liquid phase for analysis and inclusion into the biophotonic composition.

Exemplary biophotonic compositions that are useful in the methods and uses of the disclosures, include but are not limited to the below formulation.

UP Liquid Carrier

| | | Final concentration of the |
|---|---|---|
| | % w/w | actives after mixing % w/w |
| Propylene Glycol USP/EP | 56.40 | 11.28 |
| glycerin | 11.60 | 2.32 |
| Urea Peroxide | 16.50 | 3.30 |
| Disodium EDTA | 0.50 | 0.10 |
| Purified Water | 15.00 | |
| pH 4.7-4.9 with HCl solution 10% | | |
| Total | 100.00 | |

For the preparation of the above UP Liquid Carrier, the following steps were utilized. The disodium EDTA was dissolved into water at 45-50° C. The glycerin and the propylene glycol were mixed separately within the main reaction vessel. The disodium EDTA solution was then added to the main reaction vessel. The urea peroxide was dissolved and the pH of the mixture was adjusted to a pH of 4.7 to 4.9.

Chromophore Pluronic Gel

| non sterile | % w/w | |
|---|---|---|
| pluronic F127 | 23.75 | 19.00 |
| eosin | 0.0228 | 0.01820 |
| rose bengal | 0.0018 | 0.00145 |
| REISHI extract in propylene | 6.7 | 5.36 |
| glycol | | |
| Purified Water | 69.1504 | 58.32032 |
| phenoxyethanol | 0.375 | 0.30 |
| pH 5.0-5.3 | | |
| Total | 100.0000 | 100.00 |

For the preparation of the above Chromophore Pluronic Gel, the following steps were utilized. While maintaining the temperature at 4-8° C., the Pluronic F127 was slowly added to the purified water until fully dissolved. Eosin and Rose Bengal were then dissolved into the solution. Next, the Reishi extract and the phenoxyethanol were added to the mixture, and mixed until a homogenous mixture was obtained. The pH of the mixture is adjusted to a pH of 5.2 to 5.6. Finally, about 1 part UP Liquid Carrier and about 4 parts Chromophore Pluronic Gel, were mixed prior to use of the formulation. The resulting product is sometimes referred to herein as Gel X (or Pluronic EB gel).

### Example 3: Fluorescence of the Biophotonic Compositions

An assessment was performed to evaluate the capacity of gel compositions comprising fungal-derived chromophores to produce fluorescence upon being illuminated with one or more wavelengths of actinic light. The light source used for the experiments was a multi-LED lamp equipped with blue light emitting LEDs, or equipped with blue light emitting LEDs and green light emitting LEDs in a blue LED:green LED ratio of 1:1. The lamp could be set to be operated at 100% output capacity or to have the LEDs operating at an 84% output capacity (equal to a 115 mW output).

Gels were prepared to include the particular fungal-derived chromophore recipe (Gel A, Gel B or Gel C) and a xanthene dye chromophore such as Eosin Y or Rose Bengal or lacking the xanthene dye. The given gel was then illuminated using the blue or blue/green light-emitting lamp and set at the selected power output (100% or 84%) with three measurements being taken for each gel at intervals of 30 seconds during a 5 minute period of illumination. The distance between the gel surface and the light source was 5 cm for each gel.

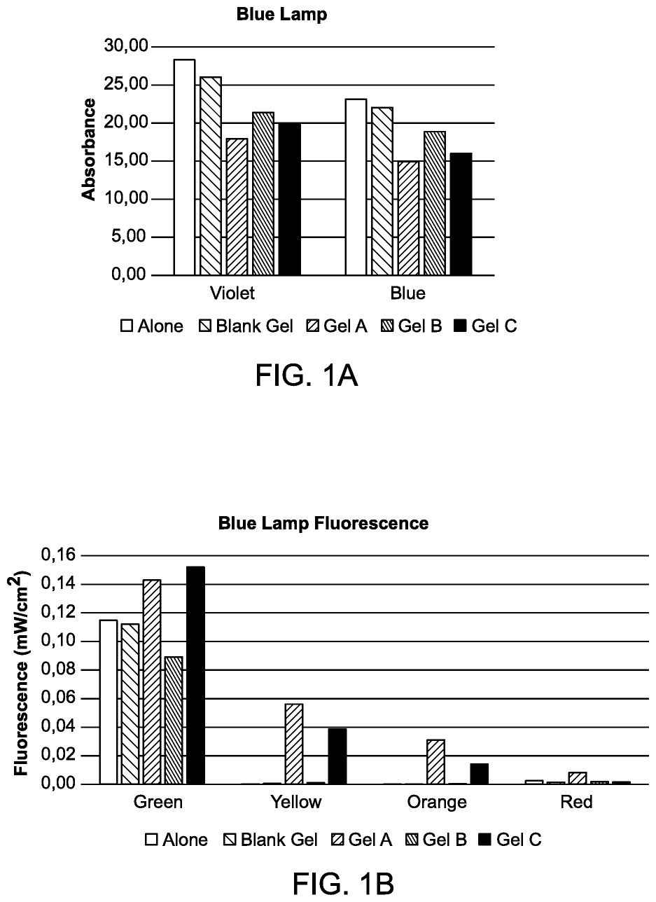

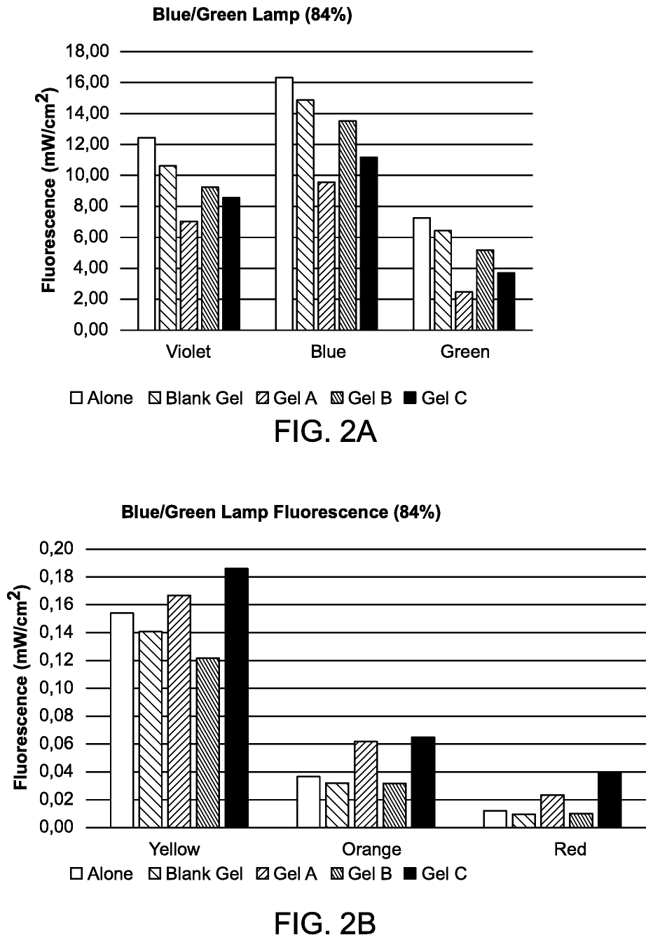

Results from the various gels illuminated with the blue light emitting lamp are presented in Table 1 below and FIG. 1, while the results from the various gels illuminated with the blue/green (50:50) light emitting lamp operating at either 84% output are presented in Table 2 and FIG. 2, respectively.

Referring to Table 1 and FIG. 1, the gels comprising either Gel A or Gel C exhibited the most influence on fluorescence being emitted from the illuminated gel. Referring to Table 2 and FIG. 2, for the various gels illuminated with the blue/green emitting lamp, the gels comprising either Gel A or Gel C also exhibited the most influence on fluorescence being emitted from the illuminated gel.

TABLE 1
Color Breakdown Blue Lamp

| Color | Alone | Blank Gel | Gel A | Gel B | Gel C |
|---|---|---|---|---|---|
| Violet | 28.31 | 26.03 | 17.93 | 21.37 | 19.99 |
| Blue | 23.13 | 22.04 | 14.93 | 18.87 | 16.00 |
| Green | 0.11 | 0.11 | 0.14 | 0.09 | 0.15 |
| Yellow | 0.00 | 0.00 | 0.06 | 0.00 | 0.04 |
| Orange | 0.00 | 0.00 | 0.03 | 0.00 | 0.01 |
| Red | 0.00 | 0.00 | 0.01 | 0.00 | 0.00 |

TABLE 2
Color Breakdown Blue/Green Lamp (84%)

| Color | Alone | Blank Gel | Gel A | Gel B | Gel C |
|---|---|---|---|---|---|
| Violet | 12.44 | 10.62 | 7.03 | 9.24 | 8.57 |
| Blue | 16.34 | 14.87 | 9.55 | 13.52 | 11.17 |
| Green | 7.25 | 6.43 | 2.47 | 5.17 | 3.70 |
| Yellow | 0.15 | 0.14 | 0.17 | 0.12 | 0.19 |
| Orange | 0.04 | 0.03 | 0.06 | 0.03 | 0.06 |
| Red | 0.01 | 0.01 | 0.02 | 0.01 | 0.04 |

### Example 4: Assay for Oxygen/Oxygen-Species (Reaction Oxygen Species (ROS)) Production

The ROS test assay is that which is described in Krumova et al. “How Lipid Unsaturation, Peroxyl Radical Partitioning, and Chromanol Lipophilic Tail Affect the Antioxidant Activity of α-Tocopherol: direct Visualization via High-Throughput Fluorescence Studies Conducted with Fluorogenic α-Tocopherol Analogues” (J. Am. Chem. Soc. 2012, vol. 134, pages 10102-10113). The assay utilizes highly sensitive fluorogenic α-tocopherol (TOH) analogues that undergo a 30-fold fluorescence intensity enhancement upon their reaction with peroxyl radicals that are generated due to the oxidation of the liposome membrane with ROS species that may be present in the reaction mixture. The assay utilizes a high-throughput microplate reader that relies on the high sensitivity of the TOH probes and provides a quantitative treatment of the temporal evolution of the fluorescence intensity thereby allowing for kinetic information to be obtained under the conditions being analyzed. The TOH analogues are two-segment receptor-reporter probes that consist of a chromanol moiety coupled to the meso position of a BODIPY fluorophore, either by an ester linker (the probe being called H₂B-TOH) or via a methylene linker (the probe being called H₂B-PMHC). The chromanol moiety quenches the emission of the fluorophore until it is oxidized following reaction with peroxyl radicals. The reporter segment for both probes is an improved BODIPY dye having improved redox potential; the favorable photoinduced electron transfer from the chromanol to the BODIPY group allows for an excellent contrast between the dark (reduced) and emissive (oxidized) states, thereby allowing for the high-throughput fluorescence method to be practiced. Both probes have been designed to ensure the efficient photoinduced electron transfer from the chromanol to the BODIPY segment, thereby ensuring an overall sensitivity to the “off-on” probe.

### Example 5: Stability of the Urea Peroxide within Various Biophotonic Compositions of the Disclosure

The stability of urea peroxide under various biophotonic compositions was evaluated. The stability of urea peroxide was tested under the following conditions: 1) urea peroxide and Pluronic; 2) urea peroxide, EDTA, and Pluronic; 3) urea peroxide and Carrier Gel; 4) 3%-12% urea peroxide and water; 5) urea peroxide and liquid carrier 20/15 or 15/15; 6) urea peroxide and liquid carrier 16.5%; 7) 15% urea peroxide in water; and 8) urea peroxide, Premix Pluronic, and EDTA. Stability of urea peroxide was assessed using the permanganate titration method. Briefly, an appropriate aliquot of H₂O₂ sample was transferred to a tared weighing bottle and weighed on an analytical balance. An appropriate aliquot of H₂O₂ was used based on Table 3 below.

TABLE 3
Estimation for use of H₂O₂ in sample

| Expected H₂O₂ | Suggested Sample |
|---|---|
| Concentration (wt. %) | Amount (grams) |
| 0.25-1.0 | 100-250 |
| 1-3 | 30-100 |
| 3-10 | 10-30 |
| 10-35 | 3-10 |
| 35-70 | 1.5-3.0 |

The weighed sample was carefully washed into a 250-mL volumetric flask with distilled water, diluted to the mark and mixed thoroughly. Pipetted a 25-mL aliquot into a 400 mL beaker containing 250-mL of distilled water and 10 mL of concentrated sulfuric acid. Titrated to the first permanent pink color with 0.3N potassium permanganate. The following calculation was performed: % H₂O₂ (by weight)=[(mLs KMnO₄)×(N)×(0.01701)×(1000)]/(grams of H₂O₂ sample used) where: N=Normality of the standardized potassium permanganate.

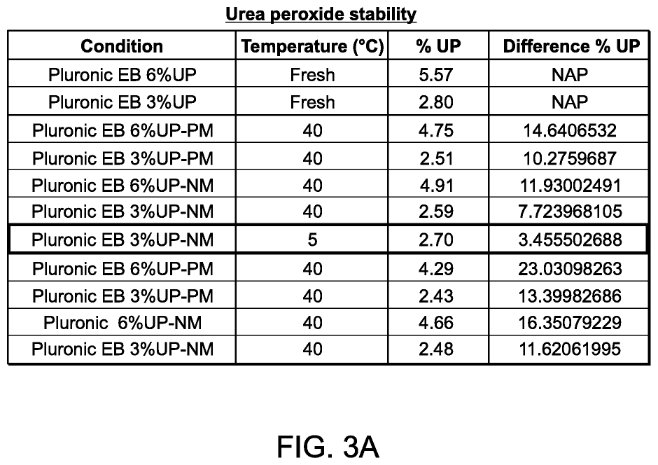

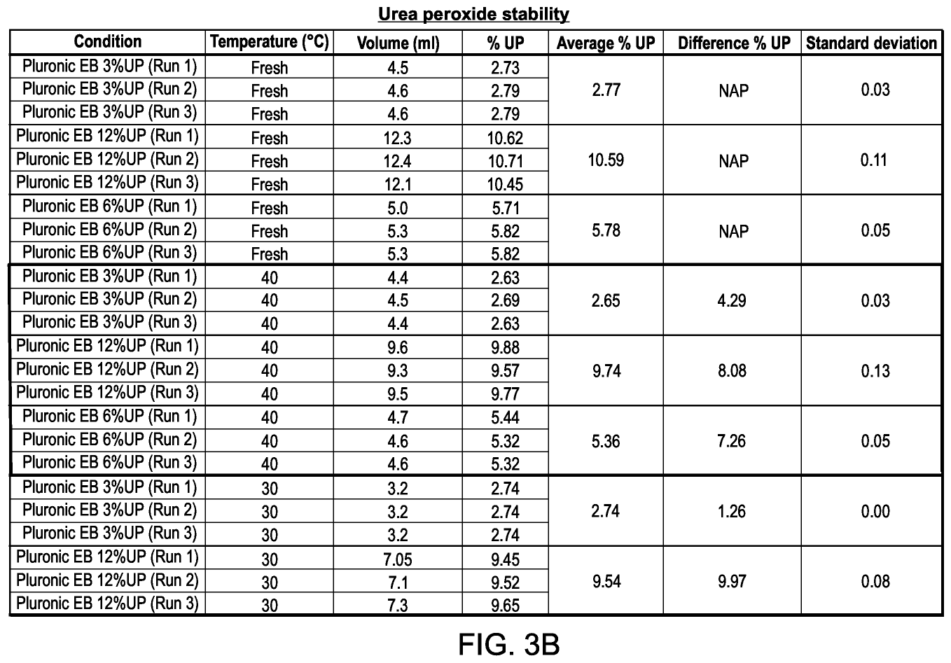

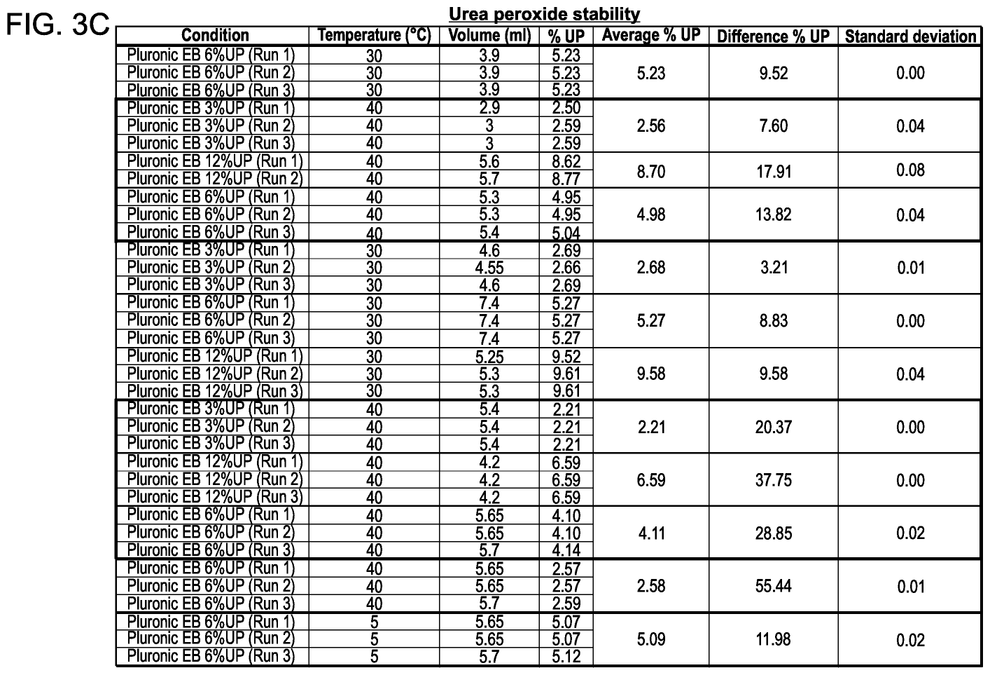

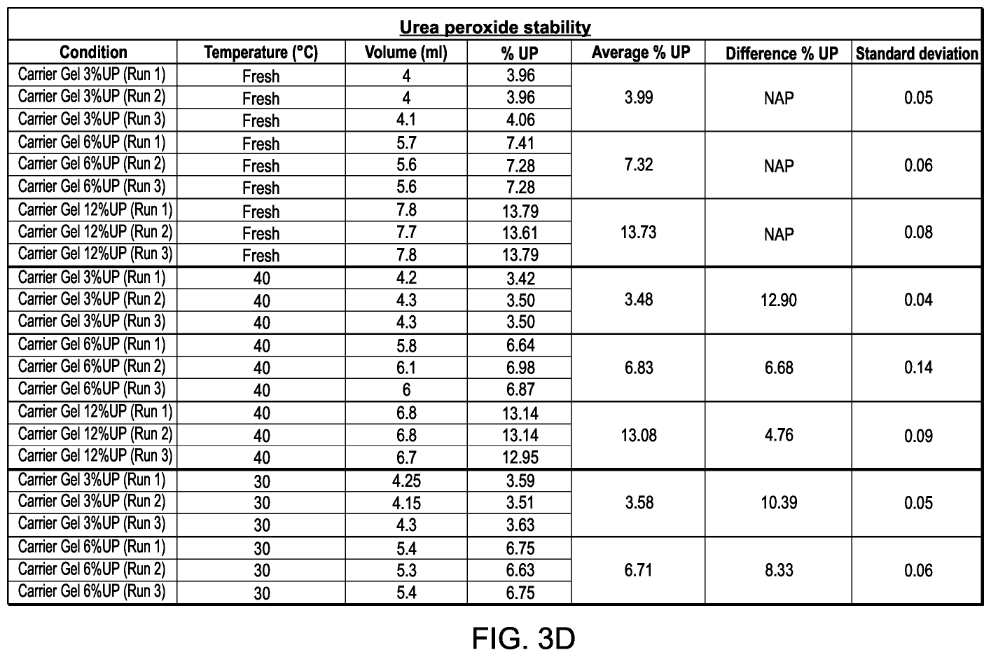

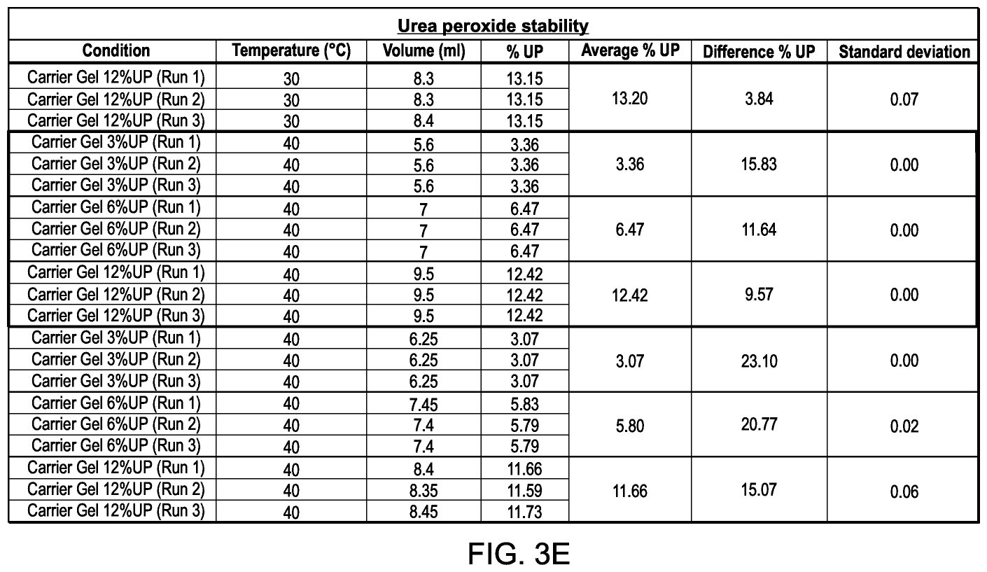

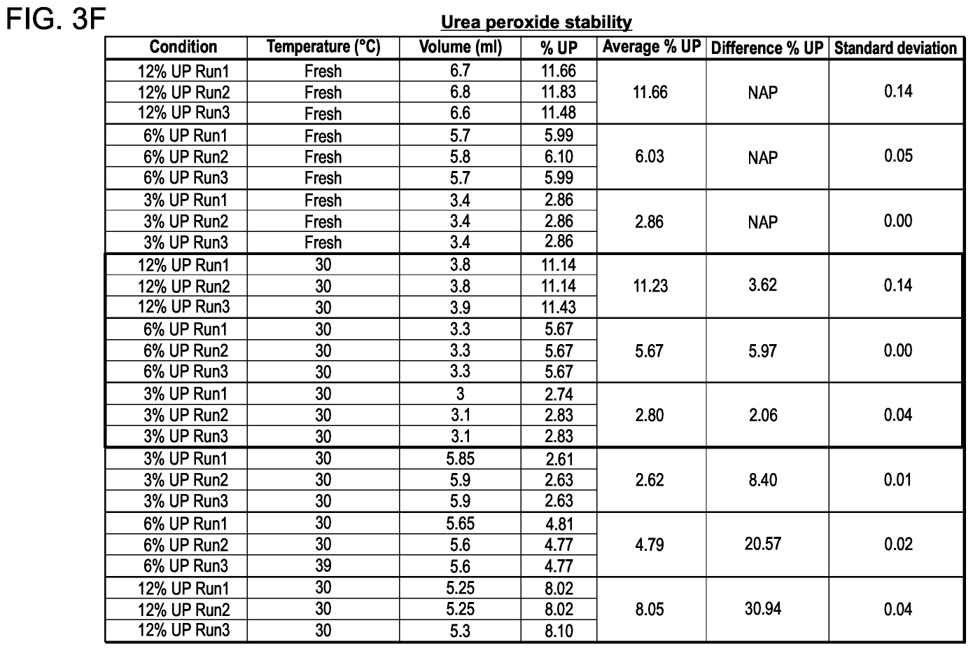

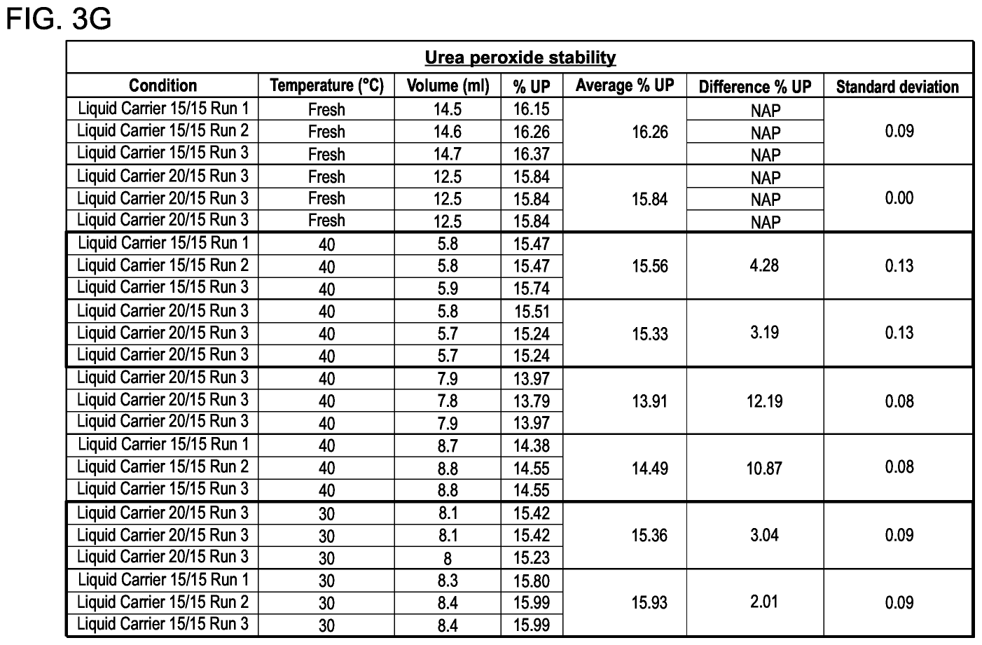

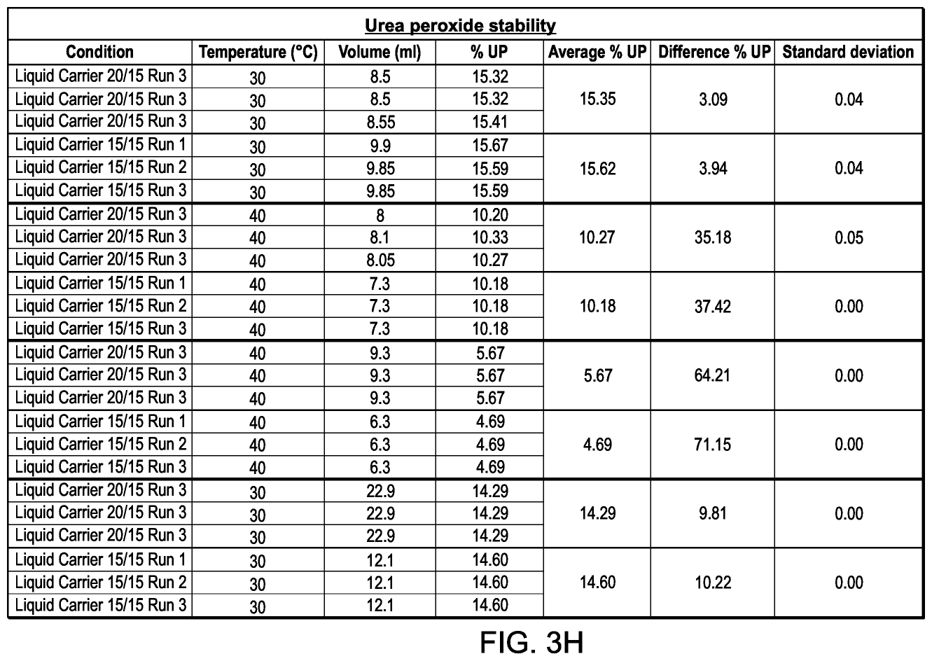

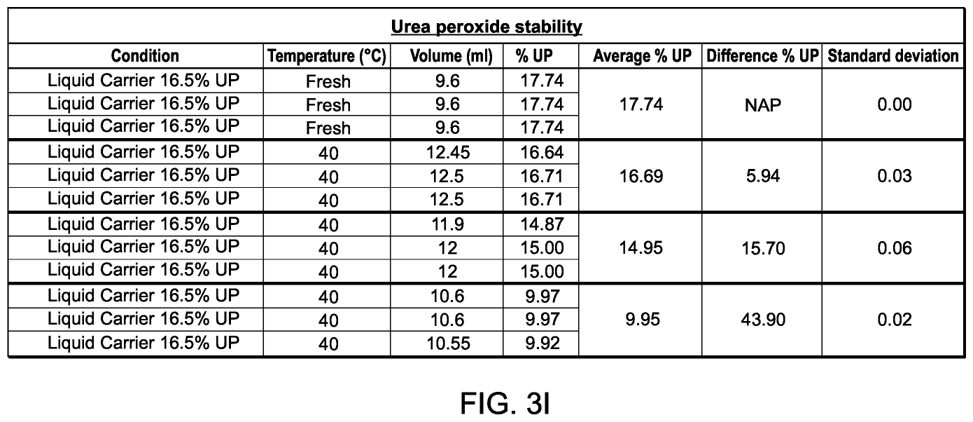

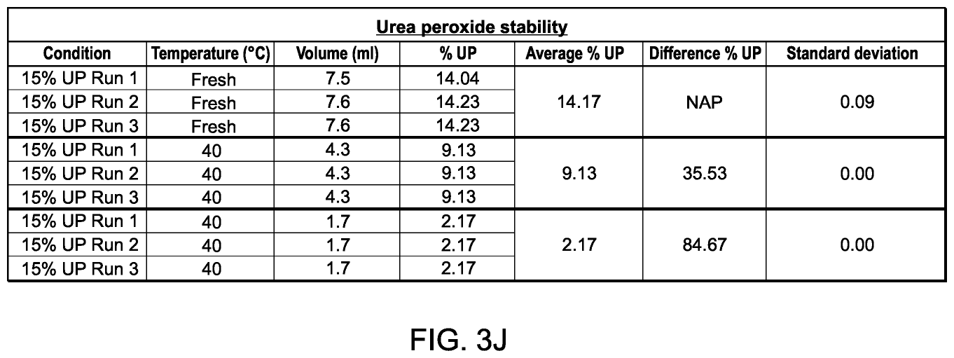

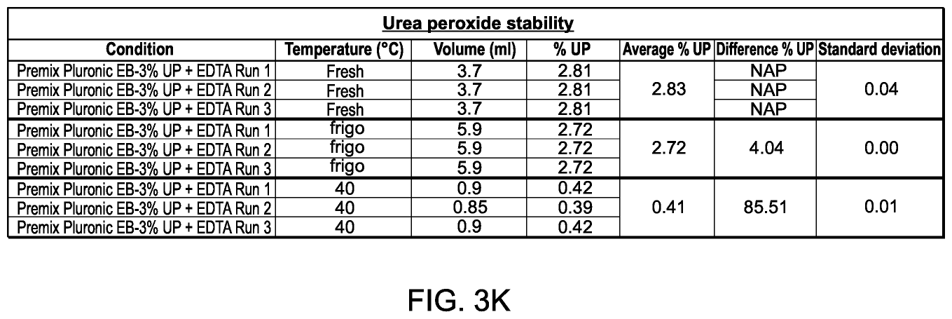

Data corresponding to each of the above conditions is shown in FIGS. 3A-3K.

### Example 6: Extraction and Determination of Palmitic Acid, Oleic Acid, Linoleic Acid and Linoeliadic Acid in Reishi Mushrooms by GC-MS

A protocol was used to analyze Reishi Mushrooms raw material by GC-MS for the presence of Palmitic Acid, Oleic Acid, Linoleic Acid and Linoeliadic Acid. Organic Gandoderma Lucidum (Reishi Mushroom) was analyzed using the following protocol:

### Standards and Reagents:

1. Polypropylene Glycol, Reagent grade (or equivalent).
2. Purified Water (USP grade)
3. Ethanol, HPLC grade (or equivalent).
4. Palmitic Acid, Sigma (or equivalent).
5. Oleic Acid, Sigma (or equivalent)
6. Linoleic Acid, Sigma (or equivalent)
7. Linoeliadic Acid, Sigma (or equivalent)

### Samples:

1. Reishi Mushrooms raw material

### Equipment:

1. Thermo Scientific ISQ110911 Mass Spectrometer
2. Thermo Scientific Trace GC 403 SSLISQ
3. RapidVap Labconco Nitrogen Evaporator
4. Analytical Balance
5. Shaker
6. Glassware as required
7. 180 μm Nylon Net Filters

### Chromatographic Conditions:

Column: Agilent GC-column DB-17MS 20 m×0.18 mm×0.18 um
Injection volume: 1 μL
Injection Mode: Split less
Injector temperature: 250° C.
Flow rate: 1.0 ml/min
Detector: MSTransfer line Temp. 280′CMS Ion Source: 280′C
Oven programmation: 40 C (3 min) then 5 C/min to 150 C (1 min) then 10 C/min to 220 ′C (5 min)
Run Time: 40 minutes
Diluting solution: Ethanol:Water (70:30) for samples Hexane for standards
Needle Rinse Solution: Hexane (if applicable)
Retention Times: Palmitic Acid (:::: 31.4 min)
Oleic Acid (:::: 33.5 min)
Linoeliadic Acid (:::: 33.6 min)
Linoleic Acid (:::: 33.7 min)

### System Suitability:

1. The number of theoretical plates for each active in the SST chromatograms should be within ±1.0% (absolute) of theoretical value of 24.37 for palmitic acid.
2. Symmetry factor (Tailing) for Salicylic Acid peaks in the SST injections should be NMT 2.0
3. % RSD on area of each for the 5 injections of the SST should be NMT 15.0%.
4. % Recovery between response of STD-2 and average response of STD-1 should be within the specification 80.0%-120.0%
   Diluent Preparation: Mixed together 70 ml of Ethanol and 30 ml of Purified water.
   Preparation of 0.5 mg/mL of Palmitic Acid: Accurately weighed and transferred about 25 mg±2.5 mg of Palmitic acid reference standard into 50 ml volumetric flask. Dissolved and completed each to volume with Hexane.
   Preparation of 0.5 mg/ml Oleic Acid Stock Standard: Accurately weighed and transferred about 25 mg±2.5 mg of Oleic acid reference standard into 50 ml volumetric flask. Dissolved and completed each to volume with Hexane. Mixed well.

Preparation of 0.5 mg/ml Linoleic Acid Standard: Accurately weighed and transferred about 25 mg±2.5 mg of Palmitic acid reference standard into 50 ml volumetric flask. Dissolved and completed each to volume with Hexane. Mixed well.

Preparation of 0.5 mg/ml Linoeliadic Acid: Accurately weighed and transferred about 25 mg±2.5 mg of Palmitic acid reference standard into 50 ml volumetric flask. Dissolved and completed each to volume with Hexane. Mixed well.

Resolution Solution Preparation:

From each stock standard preparation, the following dilutions were prepared into the same 50 ml volumetric flask and completed to volume with Hexane.

### Stock Palmitic Acid (STK-PA-1): 10.0 ml

### Stock Oleic Acid (STK-OA-1): 10.0 ml

### Stock Linoleic Acid (STK-LA-1): 10.0 ml

### Stock Linoeliadic Acid (STK-LLA-1) 10.0 ml

Target Concentrations

| | | |
|---|---|---|
| Palmitic Acid | 0.100 | mg/mL |
| O/eic Acid | 0.100 | mg/mL |
| Linoleic Acid | 0.1000 | mg/mL |
| Linoeliadic Acid | 0.100 | mg/mL |

Sample Preparation For Organic Ganoderma Lucidum (Reishi): Accurately weighed about 50.0 g of the sample into a 300 ml plastic container. Added 250.0 ml of Propylene Glycol. Shaken continuously for 5 days. Filtered the dispersed solution through 180 μm Nylon filter. Transferred 15 ml of the filtered solution to a 15 ml tube. Transferred 300 μl into evaporation tube. Evaporated using a gentle stream of nitrogen (Rapid Vap at T90 ′C). Dissolved the solid residual with the diluent and transferred the solution to a 15 ml glass tube. Rinsed the evaporation tube and completed to 10 ml with diluent preparation. Mixed well.
Filtered a portion of through a 0.45 μm nylon filter and transfer into GC-MS vial discarding the first 2 ml.

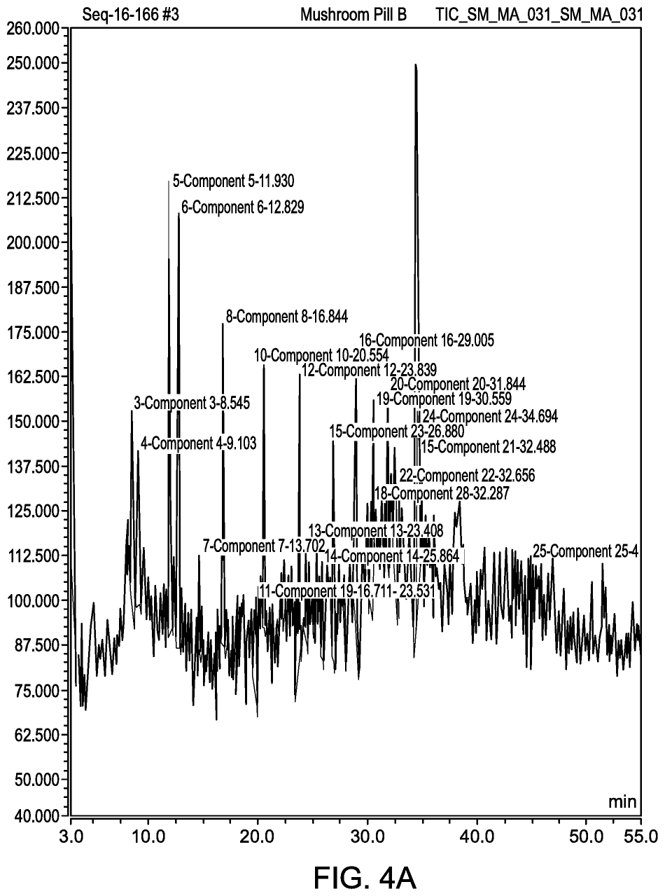

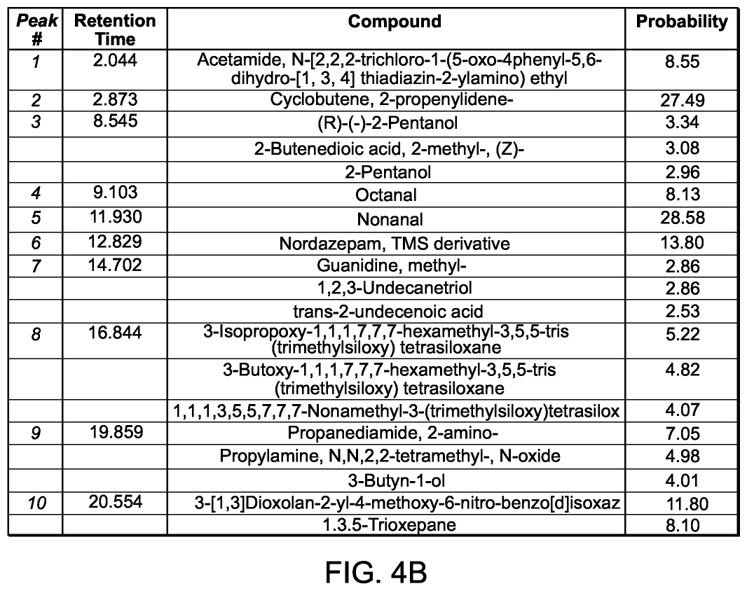

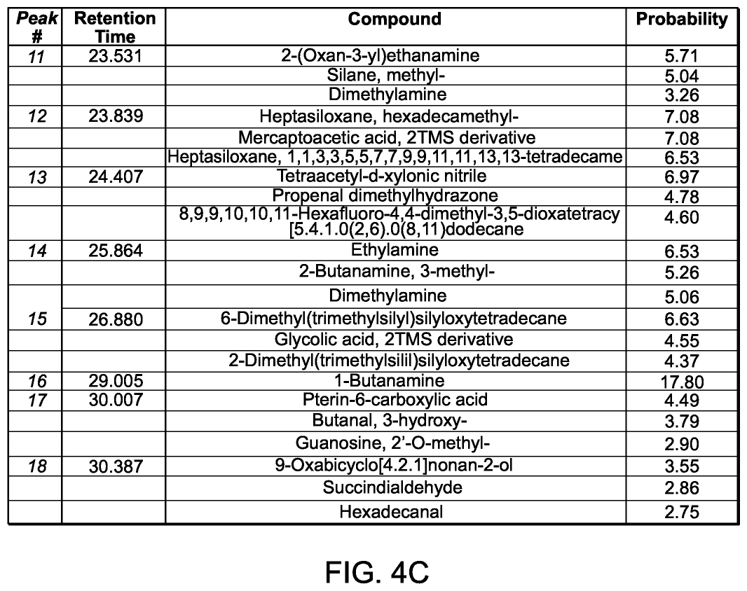

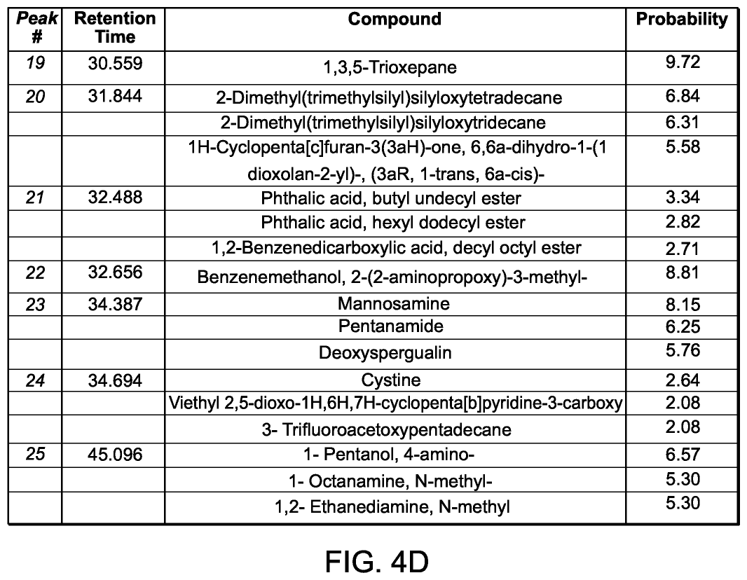

FIGS. 4A-4D provide a GC-MS Plot of Reishi extract and the corresponding data from the present study.

### Example 7: In Vitro Release (IVR) Test and Fluorescence of Chromophore Mesh

In the present study, the fluorescence and the leaching of the mesh (the photoactivatable fabric comprising strands of sheath/core configuration disclosed herein) with and without the gel superimposed were quantified. This study was also used to identify suitable mesh compositions for use with the present biophotonic compositions (e.g., the Pluronic EB gel or Gel X or other gel compositions described herein) described herein, for minimal leaching while providing maximal fluorescence.

The mesh compositions described herein were compared to a previous known mesh composition (sometimes referred to herein as “homo mesh”), described in publication WO 2016/065488 A1. The mesh composition tested in the present example comprises of a sheath of nylon surrounding a nylon core containing 1% chromophore. The sheath to core ratio examined were 25/75, 50/50, 75/25 and 10/90, wherein the first number represents the sheath by weight of nylon, and the second represents the core by weight of nylon. Other suitable ratios of sheath/core, as disclosed herein, can also be tested and confirmed for desirable characteristics (e.g. minimal leaching with maximal fluorescence). For the 10/90 sample, two manufacturers were used: Advanced Color Technologies (Source A) and Unicolor (Source B). Measurements were made on the mesh itself and with some gel superimposed on it. The gel used is the Pluronic EB gel with 3% urea peroxide and EDTA. As described herein, the Pluronic EB gel (also referred to as Gel X), refers to the combination of UP Liquid Carrier+Chromophore Pluronic Gel as detailed in Example 2 above. The Pluronic EB gel combined with the sheath/core fiber mesh composition described herein (generally referred to as the gel-mesh device) form the basis of a device, sometimes referred to herein as the gel-mesh BioPhotonic System.

### Materials and Methods

### In Vitro Release Test (IVR Test)

The release system consists of a 3-cm diameter compartment with a polycarbonate membrane with pore sizes of 3 μm at the bottom. Inside this compartment, the mesh was placed at the bottom. The Pluronic EB gel with 3% urea peroxide and EDTA was superimposed, at a thickness of 2 mm (1.4 mL approximately), when necessary. This compartment is placed inside of a well with 11 mL of phosphate buffer saline (PBS) such that the membrane just touches the surface of the solution. The sample was illuminated under the Cat-II lamp (blue-green lamp) for 10 minutes and the amount of eosin Y leached into the PBS solution was determined using the Cary Eclipse. Using eosin Y in PBS, the fluorescence intensity of each standard solution was plotted against its concentration, making a standard curve. The fluorescence intensity of the leached eosin Y was measured against the standard curve to determine its concentration.

### Lamp and Fluorescence Measurements

To measure the fluorescence, a 2-mm thick sample is placed 5 cm away from the Cat-II lamp (blue-green lamp) for 5 minutes of illumination. Below the sample, a SP-100 spectroradiometer along with a filter measured the power density spectra. The raw data corresponds to the irradiance of the sample. This plots the data against the wavelength and presents the data in a table per colour. Also, the energy observed per colour is calculated.

### Results

### In Vitro Release Test

TABLE 4
In Vitro Release Test for the Mesh with and without Gel

| | Concentration | |
|---|---|---|
| | of Eosin Y | Standard |
| Sample | (ppb) | Deviation |
| Homo Chromophore Mesh (Homo Mesh) | 27.0 | 1.41 |
| 10/90 Source A Chromophore Mesh | 26.0 | 2.83 |
| 10/90 Source B Chromophore Mesh | 45.5 | 0.71 |
| 25/75 Chromophore Mesh | BDL* | BDL |
| 50/50 Chromophore Mesh | BDL | BDL |
| 75/25 Chromophore Mesh | BDL | BDL |
| Homo Chromophore Mesh + Gel | 96.0 | 7.07 |
| 10/90 Source A Chromophore Mesh + Gel | 95.5 | 21.92 |
| 10/90 Source B Chromophore Mesh + Gel | 125.0 | 7.07 |
| 25/75 Chromophore Mesh + Gel | 141.0 | 14.14 |
| 50/50 Chromophore Mesh + Gel | 182.0 | 28.28 |
| 75/25 Chromophore Mesh + Gel | 197.0 | 21.92 |

*BDL = Below Detection Limit (5 ppb)

The in vitro release test shows that there is more leaching when the gel was layered on top of the mesh. Without the gel, only the 10/90 chromophore mesh and the homo mesh were quantifiable. All the other ratios are below the detection limit. This suggests that a minimum ratio of 25/75 sheath to core is desirable to prevent any eosin from leaching out of the nylon core. With the gel, a higher concentration of eosin Y was detected as the sheath got larger. Most of the eosin Y detected is most likely from the gel. This suggests that a larger sheath increases the likelihood that chromophore from the gel leaches through the mesh and into solution. Pore size variability could be a factor. It may get slightly larger as the sheath size increases.

After 24 hours, leaching occurs for the 25/75 chromophore mesh and the 50/50 chromophore mesh. For the 75/25 chromophore mesh, no detectable amount of eosin Y was observed. Thus, if the mesh is to be in contact with skin for more than 24 hours, the 75/25 mesh is desirable.

### Fluorescent Measurements

In general, adding the gel on top of the mesh decreased the amount of fluorescence observed. Without wishing to be bound by any theory, this may be due to the process itself. Adding a gel on top may create an additional barrier between the light source and the mesh. Hence, most of the light is likely absorbed by the gel and less light may be available to excite the mesh to emit fluorescence. Even though fluorescence is lower when the mesh and gel are used together, it is still much higher than the gel alone. Thus, the mesh contributes significantly to the fluorescence even though less light reaches it.

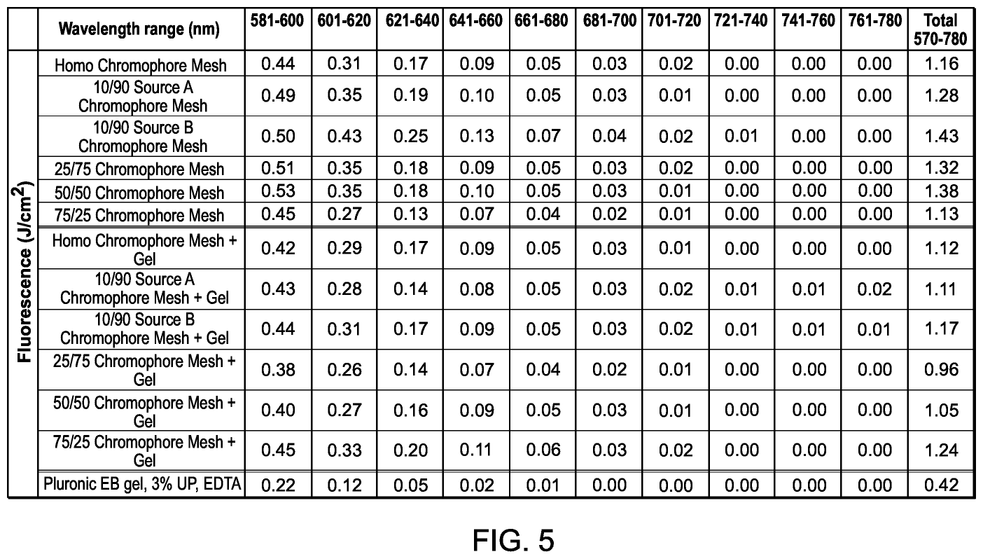

With or without the gel, no correlation exists between the size of the sheath and the fluorescence. This is unexpected since an increase in sheath size decreases the number of chromophores present. It would be expected that a larger sheath would correlate with less fluorescence. However, no correlation seems to exist between sheath size and fluorescence. FIG. 5 summarizes the fluorescence breakdown of Samples.

Overall, if the mesh is to be used as is, the 50/50 chromophore mesh should be used since it has the highest amount of fluorescence when no chromophore is quantified after leaching for 10 minutes. If leaching is not an issue, the 10/90 chromophore mesh can be used as well. If the mesh is to be used with the gel, the 10/90 chromophore mesh should be used to minimize leaching and maximize fluorescence. If the mesh is to be used for periods longer than 10 minutes, the 75/25 chromophore mesh should be used as it does not leach.

The Orphaderm BPS (generally, the gel-mesh BioPhotonic System) described herein was used in evaluating skin sensitization potential in mice using the Local Lymph Node Assay (LLNA). Specifically, skin sensitization potential of the gel-mesh BPS (Pluronic EB gel+sheath/core fiber fabric) along with a multi-LED lamp (e.g., delivering non-coherent blue light with peak wavelengths at 447 nm and non-coherent green light with peak wavelengths at 521 nm) was administered daily by dermal application on the dorsal surface of the pinna of both ears to mice for 3 days. The gel-mesh BPS was shown to have no sensitization potential following dermal application on the dorsal surface of the pinna of both ears for 10 minutes/day for 3 consecutive days in the LLNA in mice, and is thus characterized as a non-sensitizer (data not shown).

### Example 8: Epidermolysis Bullosa Clinical Trial Design and Study

Overview of Study Design

Described herein is a prospective, randomized, controlled, assessor-blinded study. Two clinical sites will be used: adult patients (18 and older—located in Europe); and pediatric patients (ages 6-18—location to be determined). Ten patients suffering from Epidermolysis bullosa (any subtype) will be recruited: patients of 18 years and older in the first site (5 patients), and patients ages 6-18 in the second site (5 patients), with a minimum of 20% of the patients with Simplex subtype, and 20% with Dystrophic subtype. Each patient will have a maximum of two wound areas selected by the investigator according to eligibility criteria. Once these wound areas are selected, they will be randomly assigned to one of these treatment groups: 1) Treatment with Orphaderm BioPhotonic System (gel-mesh BPS) described herein twice a week+SOC; 2) SOC. The study will include an expert's review of the pictures during the screening period, to confirm that the wounds meet eligibility criteria. Efficacy results (wound size and incidence of wound breakdown) will be assessed by a Blinded Experts Committee (on pictures). Statistical significance will not be needed for this study.

Study Procedures Screening: One week (7 days±2) before the first treatment visit. Run-in period: exclusion of the wound if decrease of more than 30% with SOC (if increase of more than 30%, the wound will be accepted). Pictures will be assessed by an independent expert for confirmation of inclusion during the screening period. Treatment period will continue until total closure of the targeted wound area selected to be treated with gel-mesh BPS, with a maximum of 16 weeks. Follow-up period will be 4 weeks. If the wound is not closed after the follow-up period, possibility to treat with Orphaderm the wounds in the SOC group (to be determined by investigator).

Primary Endpoint—Efficacy

To document the efficacy of the gel-mesh BioPhotonic System compared to SOC in the treatment of Epidermolysis bullosa on a target wound, the percentage change of the target wound area during the study period (Treatment period+Follow-up period) will be assessed. Assessments through a specific Imaging system will be conducted twice a week.

Secondary Endpoint—Efficacy

Various secondary endpoints will be used, including determining the percentage of target wounds that reach a total closure during the study period (Treatment period+Follow-up period); mean time to complete target wound closure; or incidence of wound breakdown two weeks after wound closure (for wounds that achieved total wound closure). Incidence of wound breakdown will be assessed by a physician.

Secondary Endpoint—Safety

To document the safety and tolerability of the gel-mesh BioPhotonic System in the treatment of Epidermolysis bullosa, the following will be monitored: Related AEs/SAEs/Incidents during the treatment period and the 4-week follow-up period; labs (Haematology, Biochemical); frequency of wound infection requiring a systemic antibiotic therapy (see Discontinuation criteria, described herein, if the patient is treated with antibiotics). Safety will be assessed up to four weeks after the last treatment visit. Also, change in wound pain linked to the target wound (using Visual Analog Scale—VAS), and/or change in wound itching on target wound (using a 0 to 10 Itching scale) will be assessed.

Exploratory Endpoints

Factors influencing exploratory endpoints will include one or more of the following: change in perilesional skin (e.g., reduction of erythema); patients' questionnaire (e.g., perception of the treatment, time to wound closure compared to the time usually observed); impact of the treatment on the patients' Quality of life (using CWIS—Cardiff Wound Impact Schedule); and/or absence of wound breakdown four weeks after wound closure. Incidence of wound breakdown will be assessed by a physician.

Inclusion Criteria at Screening Visit

All patients will have signed and dated a written informed consent form. Patients will have received a histologically confirmed diagnosis of Epidermolysis bullosa (of any sub-type). EB patients will have at least two wound areas (size of each wound area: 10 to 216 cm²): 1) Possibility to use two adjacent meshes (dimensions of the mesh: 10×14 cm; 2×10 cm×14 cm=216 cm²); mesh should cover in periphery a minimum of 1 cm of healthy skin all around the wound to be treated, such that the maximum dimensions of the wound should be: 18×12 cm=216 cm².

The wound areas selected (target wound area and control wound area) have to be: present for at least 2 weeks before the Screening visit; of comparable severity; with comparable sizes (no more than 50% difference between the two wounds); separated by a distance of at least 10 cm of healthy tissue; if possible, on a flat surface; if possible, symmetrical wounds or wounds present on different body regions (e.g., symmetric limb or on another body region on the same hemi-quadrant); if the wounds do not meet these preferred criteria, they will need the independent expert's approval on pictures before their inclusion in the study.

Generally, if several wounds are present in a selected wound area, the largest and more comparable wound will be selected. Both male and female patients should be willing to adhere to a medically-accepted birth control method during the course of the study, if appropriate and applicable. Patients should be willing to comply with study requirements (e.g., visits, treatments).

Inclusion Criteria at First Treatment Visit, Before any Treatment

An independent expert will validate the inclusion of a patient based on pictures of the wounds taken at the Screening visit. Patients showing an absence of decrease of the size area of the selected wounds of more than 30% during the run-in period will be included.

Exclusion Criteria

Patients will be excluded based on one or more of the following criteria: patient does not meet all inclusion criteria; pregnant or breastfeeding women; wound areas greater than 216 cm² (or with a maximum length of more than 18 cm) that are located on a flexural surface or on a mucous membrane; wounds present on ankles, toes and fingers; wounds present on feet; overlapping wounds; clinical signs of infection of the target wound area (the patient is however eligible for re-screening after the systemic infection has subsided); no clinical evidence suggesting a local neoplasic change or a squamous cell carcinoma in the wound areas selected; absence of any other dermatological disease that may adversely impact wound healing or interfere with assessment of efficacy according to investigators; any chronic medical condition that could interfere with the study treatment according to the investigator; patient taking drugs or products or with conditions known to induce severe photosensitivity reactions; use of topical immunomodulators; use of systemic or topical steroids within 7 days prior to enrollment; use of systemic antibiotics within 7 days prior to enrollment.

Discontinuation Criteria

Use of the following products or drugs will not be allowed during the study period: drugs or products known to induce severe photosensitivity reactions; topical immunomodulators on the selected wounds; systemic steroids; topical steroids on the selected wounds; systemic antibiotics; topical antibiotics on the selected wounds. If the use of one of these products or drugs is deemed necessary for medical reasons during the study period, the treatment with gel-mesh BPS should be immediately interrupted and the sponsor must be informed. A therapeutic window of a maximum of two weeks is authorized during the treatment period. Once the unauthorized treatment is completed within this therapeutic window, the investigator may ask a waiver to the sponsor to continue the study and reintroduce the treatment with gel-mesh BPS.

Treatment Procedure

Treatment procedure comprises the following: dressing/bandages removal; cleansing of the wound with saline water; application of the mesh; application of the gel (1.5 mm thickness) directly on the mesh; illumination with the lamp (e.g., CAT II lamp) for 10 minutes; removal of the mesh; removal of the gel by the specific removal method specified in the IFU; new dressing/bandages according to SOC. Untreated areas around the wounds will be protected by a cloth during illumination by the lamp.

### Standard of Care

Generally, the standard of care will be adapated to the age of the patients. Standard of Care will be standardized as much as possible, with respect to, e.g., bandages and dressings, and the use of non-adherent dressings. Frequency of dressing change will depend on exudate. Blistering will be prevented by skin moisturization, soft clothing, and/or moderate temperature. Careful wound care will be maintained with regular dressing changes—frequent bandaging in order to keep blisters clean and protected is a very essential and important aspect of EB care.

Systemic treatments of pain reduction will be allowed for the management of pain. Pruritus management will be achieved by, e.g., bathing (salt++) and/or systemic treatments. Nutritional support will be maintained by providing good nutrition, with supplementation in iron, calcium and vitamin D Psychological support will also be provided. Various symptomatic treatments (e.g., dental, ophthalmologic) will also be provided.

### Example 9: Treatment of a Patient Afflicted with Epidermolysis Bullosa with a Biophotonic Composition of the Disclosure

An exemplary biophotonic composition of the disclosure is prepared by mixing a fungal-derived chromophore and a carbopol carrier gel comprising urea peroxide, as described herein. The resulting composition is applied to a tissue of a patient afflicted with epidermolysis bullosa and activated with actinic light provided by a LED photocuring device. The composition is removed following treatment.

### INCORPORATION BY REFERENCE

All references cited in this specification, and their references, are incorporated by reference herein in their entirety where appropriate for teachings of additional or alternative details, features, and/or technical background.

### EQUIVALENTS

While the disclosure has been particularly shown and described with reference to particular embodiments, it will be appreciated that variations of the above-disclosed and other features and functions, or alternatives thereof, may be desirably combined into many other different systems or applications. Also, that various presently unforeseen or unanticipated alternatives, modifications, variations or improvements therein may be subsequently made by those skilled in the art which are also intended to be encompassed by the following embodiments.

What is claimed is:

1. A biophotonic composition comprising at least one fungal-derived chromophore and a carrier medium.

2. The biophotonic composition of claim 1, wherein the at least one fungal-derived chromophore is derived from Grifola frondosa.

3. The biophotonic composition of claim 1 or 2, wherein the at least one fungal-derived chromophore is derived from a Ganoderma species.

4. The biophotonic composition of any one of claims 1 to 3, wherein the at least one fungal-derived chromophore is derived from Laricifomes officinalis.

5. The biophotonic composition of any one of claims 1 to 4, wherein the at least one fungal-derived chromophore is derived from an Agaricus species.

6. The biophotonic composition of any one of claims 1 to 5, wherein the at least one fungal-derived chromophore is derived from a Tricholoma species.

7. The biophotonic composition of any one of claims 1 to 6, wherein the at least one fungal-derived chromophore is derived from a Cordyceps species.

8. The biophotonic composition of any one of claims 1 to 7, wherein the at least one fungal-derived chromophore is derived from a Lentinula species.

9. The biophotonic composition of any one of claims 1 to 8, further comprising an oxidant.

10. The biophotonic composition of claim 9, wherein the oxidant is selected from the group consisting of hydrogen peroxide, carbamide peroxide, benzoyl peroxide, peroxy acid, alkali metal peroxide, alkali metal percarbonate, peroxyacetic acid, alkali metal perborate, methyl ethyl ketone peroxide, and combinations thereof.

11. The biophotonic composition of claim 10, wherein the oxidant is carbamide peroxide.

12. The biophotonic composition of any one of claims 1 to 11, wherein the carrier medium comprises one or more of a hydrophilic polymer, a hygroscopic polymer, or a hydrated polymer.

13. The biophotonic composition of any one of claims 1 to 11, wherein the carrier medium comprises carboxylic functional groups.

14. The biophotonic composition of any one of claims 1 to 13, wherein the carrier medium comprises one or more of a synthetic polymer selected from the group consisting of a vinyl polymer, a polyoxyethylene-polyoxypropylene copolymer, poly(ethylene oxide), an acrylamide polymer and derivatives or salts thereof.

15. The biophotonic composition of claim 14, wherein the carrier medium comprises one or more of a vinyl polymer selected from the group consisting of polyacrylic acid, polymethacrylic acid, poly hydroxyethyl methacrylate, polyvinyl pyrrolidone, and polyvinyl alcohol.

16. The biophotonic composition claim 15, wherein the vinyl polymer is selected from the group consisting of Carbopol® 940, Carbopol® 980, ETD 2020 NF, Carbopol® 1382 Polymer, 71G NF, 971P NF, 974P NF, 980 NF, 981 NF, 5984 EP, ETF 2020 NF, ultrez 10 NF, ultrez 20, ultrez 21, 1342 NF, 934 NF, 934P NF, 940 NF, or 941 NF, or combinations thereof.

17. The biophotonic composition of any one of claims 1 to 16, wherein the carrier medium comprises a protein-based polymer.

18. The biophotonic composition of claim 17, wherein the protein-based polymer is gelatin, collagen, or both.

19. The biophotonic composition of any one of claims 1 to 18, wherein the carrier medium comprises a polysaccharide.

20. The biophotonic composition of claim 19, wherein the polysaccharide is one or more of starch, chitosan, chitin, agar, an alginate, xanthan, carrageenan, guar gum, gellan gum, pectin, or locust bean gum.

21. The biophotonic composition of any one of claims 1 to 20, wherein the carrier medium comprises at least one glycol.

22. The biophotonic composition of claim 21, wherein the glycol is selected from the group consisting of ethylene glycol and propylene glycol.

23. The biophotonic composition of any one of claims 1 to 22, wherein the at least one fungal-derived chromophore is a fluorescent chromophore.

24. The biophotonic composition of claim 23, wherein the at least one fungal-derived chromophore absorbs and/or emits light within the visible range.

25. The biophotonic composition of claim 23, wherein the at least one fungal-derived chromophore absorbs and/or emits light within the green, orange and yellow portions of the electromagnetic spectrum.

26. The biophotonic composition of any one of claims 1 to 25, wherein the composition further comprises at least a second chromophore.

27. The biophotonic composition of claim 26, wherein the at least one fungal-derived chromophore has an emission spectrum that overlaps at least 20% with an absorption spectrum of the at least second chromophore.

28. The biophotonic composition of claim 26, wherein the at least one fungal-derived chromophore has an absorption spectrum that overlaps at least 20% with an emission spectrum of the at least second chromophore.

29. The biophotonic composition of claim 26, wherein the at least one fungal-derived chromophore transfers energy to the at least second chromophore upon illumination with a light.

30. The biophotonic composition of claim 26, wherein the at least second chromophore transfers energy to the at least one fungal-derived chromophore upon illumination with light.

31. The biophotonic composition of any one of claims 26 to 30, wherein the at least second chromophore is a xanthene dye.

32. The biophotonic composition of claim 31, wherein the xanthene dye is Eosin Y, Eosin B, Erythrosin B, Fluorescein, Rose Bengal, Phloxin B, or combinations thereof.

33. The biophotonic composition of claim 32, wherein the xanthene dye is Eosin Y.

34. The biophotonic composition of claim 32, wherein the xanthene dye is Rose Bengal.

35. The biophotonic composition of any one of claims 1 to 34, wherein the at least one fungal-derived chromophore is present in an amount of between about 0.0001% to about 40% by weight of the total composition, or between about 0.0001% to about 2% by weight of the total composition.

36. The biophotonic composition of any one of claims 1 to 35, wherein the biophotonic composition has a translucency of at least about 40%, about 50%, about 60%, about 70%, about 80%, about 90% or about 100% in a visible range without the chromophore(s).

37. The biophotonic composition of any one of claims 1 to 36, wherein upon exposure to light, the composition emits at least 1.25×, 1.5×, 1.75× or 2× more red, yellow and/or orange light than a composition lacking the at least one fungal-derived chromophore.

38. Use of the biophotonic composition of any of claims 1 to 37, for treatment of a rare disease that afflicts skin or soft tissues.

39. The use of claim 38, wherein the rare disease is selected from Hailey-Hailey syndrome, epidermolysis bullosa, CHILD syndrome, dermatomyositis, hidradenitis suppurativa, acquired ichthyosis, hereditary ichthyosis, lichen myxedematosus, scleromyxedema, pemphigus, a porphyria disorders, Ehlers-Danlos syndrome, cutis hyperelastica, eosinophilic fasciitis, osteogenesis imperfect, scleroderma, and Winchester syndrome.

40. The use of claim 39, wherein the rare disease is Hailey-Hailey syndrome.

41. The use of claim 39, wherein the rare disease is epidermolysis bullosa.

42. The use of claim 39, wherein the rare disease is hidradenitis suppurativa.

43. The use of claim 39, wherein the rare disease is scleroderma.

44. A method for biophotonic treatment of a rare disease that afflicts skin or soft tissues comprising:
applying a biophotonic composition to a target tissue, wherein the biophotonic composition comprises at least one fungal-derived chromophore and a carrier medium, and
illuminating said biophotonic composition with light that is absorbed by the at least one fungal-derived chromophore.

45. The method of claim 44, wherein the rare disease is selected from Hailey-Hailey syndrome, epidermolysis bullosa, CHILD syndrome, dermatomyositis, hidradenitis suppurativa, acquired ichthyosis, hereditary ichthyosis, lichen myxedematosus, scleromyxedema, pemphigus, a porphyria disorders, Ehlers-Danlos syndrome, cutis hyperelastica, eosinophilic fasciitis, osteogenesis imperfect, scleroderma, and Winchester syndrome.

46. The method of claim 45, wherein the rare disease is Hailey-Hailey syndrome.

47. The method of claim 45, wherein the rare disease is epidermolysis bullosa.

48. The method of claim 45, wherein the rare disease is hidradenitis suppurativa.

49. The method of claim 45, wherein the rare disease is scleroderma.

50. The method of any one of claims 44 to 49, wherein the at least one fungal-derived chromophore is derived from Grifola frondosa.

51. The method of any one of claims 44 to 50, wherein the at least one fungal-derived chromophore is derived from a Ganoderma species.

52. The method of any one of claims 44 to 51, wherein the at least one fungal-derived chromophore is derived from Laricifomes officinalis.

53. The method of any one of claims 44 to 52, wherein the at least one fungal-derived chromophore is derived from an Agaricus species.

54. The method of any one of claims 44 to 53, wherein the at least one fungal-derived chromophore is derived from a Tricholoma species.

55. The method of any one of claims 44 to 54, wherein the at least one fungal-derived chromophore is derived from a Cordyceps species.

56. The method of any one of claims 44 to 55, wherein the at least one fungal-derived chromophore is derived from a Lentinula species.

57. The method of any one of claims 44 to 56, wherein the composition further comprises an oxidant.

58. The method of claim 57, wherein the oxidant is selected from hydrogen peroxide, carbamide peroxide, benzoyl peroxide, a peroxy acid, an alkali metal peroxide, an alkali metal percarbonate, a peroxyacetic acid, an alkali metal perborate, methyl ethyl ketone peroxide, and combinations thereof.

59. The method of claim 58, wherein the peroxide is carbamide peroxide.

60. The method of any one of claims 44 to 59, wherein the carrier medium comprises a synthetic polymer selected from the group consisting of a vinyl polymer, a polyoxyethylene-polyoxypropylene copolymer, poly(ethylene oxide), an acrylamide polymer, a methacrylate polymer and derivatives and salts thereof and combinations thereof.

61. The method of claim 60, wherein the synthetic polymer is selected from the group consisting of Carbopol® 940, Carbopol® 980, ETD 2020 NF, Carbopol® 1382 Polymer, 71G NF, 971P NF, 974P NF, 980 NF, 981 NF, 5984 EP, ETF 2020 NF, ultrez 10 NF, ultrez 20, ultrez 21, 1342 NF, 934 NF, 934P NF, 940 NF, 941 NF, and combinations thereof.

62. The method of any one of claims 44 to 61, wherein the carrier medium comprises a protein-based polymer comprising gelatin or collagen, or both.

63. The method of any one of claims 44 to 62, wherein the carrier medium comprises a polysaccharide selected from the group consisting of starch, chitosan, chitin, agar, an alginate, xanthan, carrageenan, guar gum, pectin, locust bean gum, and combinations thereof.

64. The method of any one of claims 44 to 63, wherein the carrier medium comprises at least one glycol selected from ethylene glycol and propylene glycol.

65. The method of any one of claims 44 to 64, wherein the at least one fungal-derived chromophore is a fluorescent chromophore.

66. The method of any one of claims 44 to 65, wherein the at least one fungal-derived chromophore absorbs and/or emits light within the visible range.

67. The method of claim 66, wherein the at least one fungal-derived chromophore absorbs and/or emits light within the green, orange and yellow portions of the electromagnetic spectrum.

68. The method of any one of claims 44 to 67, wherein the composition further comprises at least a second chromophore.

69. The method of any one of claims 44 to 68, wherein upon exposure to light, the composition emits at least 1.25×, 1.5×, 1.75× or 2× more red, yellow and/or orange light than a composition lacking the at least one fungal-derived chromophore.

70. The method of claim 68, wherein the at least one fungal-derived chromophore has an emission spectrum that overlaps at least 20% with an absorption spectrum of the at least second chromophore.

71. The method of claim 68, wherein the at least one fungal-derived chromophore transfers energy to the at least second chromophore upon illumination with a light.

72. The method of claim 68, wherein the at least one fungal-derived chromophore has an absorption spectrum that overlaps at least 20% with an emission spectrum of the at least second chromophore.

73. The method of claim 72, wherein the at least second chromophore transfers energy to the at least one fungal-derived chromophore upon illumination with a light.

74. The method of any one of claims 68 to 73, wherein the at least second chromophore is a xanthene dye.

75. The method of claim 74, wherein the xanthene dye is Eosin Y, Eosin B, Erythrosin B, Fluorescein, Rose Bengal, Phloxin B, or combinations thereof.

76. The method of claim 75, wherein the xanthene dye is Eosin Y.

77. The method of claim 75, wherein the xanthene dye is Rose Bengal.

78. The method of any one of claims 44 to 77, wherein the at least one fungal-derived chromophore is present in an amount of between about 0.0001% to about 40% by weight of the total composition, or between about 0.0001% to about 2% by weight of the total composition.

79. A kit comprising the biophotonic composition of any one of claims 1 to 37.

80. The biophotonic composition of any one of claims 1 to 37, further comprising a chromophore-protecting agent.

81. A biophotonic composition prepared by the following steps:
a) Pulverizing at least one fungal source to provide a semi-fine, homogenous powder;
b) Adding five-fold excess of propylene glycol to said homogenous powder;
c) Stirring the resulting solution from step b) at low speed for at least 15 days;
d) Filtering the solution of step c) to obtain an extract comprising at least one fungal-derived chromophore; and
e) Combining the extract with a carrier medium.

82. The biophotonic composition of claim 81, wherein the at least one fungal source comprises Grifola frondosa.

83. The biophotonic composition of claim 81 or 82, wherein the at least one fungal source comprises a Ganoderma species.

84. The biophotonic composition of any one of claims 81 to 83, wherein the at least one fungal source comprises Laricifomes officinalis.

85. The biophotonic composition of any one of claims 81 to 84, wherein the at least one fungal source comprises an Agaricus species.

86. The biophotonic composition of any one of claims 81 to 85, wherein the at least one fungal source comprises a Tricholoma species.

87. The biophotonic composition of any one of claims 81 to 86, wherein the at least one fungal source comprises a Cordyceps species.

88. The biophotonic composition of any one of claims 81 to 87, wherein the at least one fungal source comprises a Lentinula species.

89. The biophotonic composition of any one of claims 81 to 88, further comprising an oxidant.

90. The biophotonic composition of claim 89, wherein the oxidant is selected from the group consisting of hydrogen peroxide, carbamide peroxide, benzoyl peroxide, peroxy acid, alkali metal peroxide, alkali metal percarbonate, peroxyacetic acid, alkali metal perborate, methyl ethyl ketone peroxide, and combinations thereof.

91. The biophotonic composition of claim 90, wherein the oxidant is carbamide peroxide.

92. The biophotonic composition of any one of claims 81 to 91, wherein the carrier medium comprises one or more of a hydrophilic polymer, a hygroscopic polymer, or a hydrated polymer.

93. The biophotonic composition of any one of claims 81 to 91, wherein the carrier medium comprises carboxylic functional groups.

94. The biophotonic composition of any one of claims 81 to 93, wherein the carrier medium comprises one or more of a synthetic polymer selected from the group consisting of a vinyl polymer, a polyoxyethylene-polyoxypropylene copolymer, poly(ethylene oxide), an acrylamide polymer and derivatives or salts thereof.

95. The biophotonic composition of claim 94, wherein the carrier medium comprises one or more of a vinyl polymer selected from the group consisting of polyacrylic acid, polymethacrylic acid, poly hydroxyethyl methacrylate, polyvinyl pyrrolidone, and polyvinyl alcohol.

96. The biophotonic composition claim 95, wherein the vinyl polymer is selected from the group consisting of Carbopol® 940, Carbopol® 980, ETD 2020 NF, Carbopol® 1382 Polymer, 71G NF, 971P NF, 974P NF, 980 NF, 981 NF, 5984 EP, ETF 2020 NF, ultrez 10 NF, ultrez 20, ultrez 21, 1342 NF, 934 NF, 934P NF, 940 NF, or 941 NF, or combinations thereof.

97. The biophotonic composition of any one of claims 81 to 96, wherein the carrier medium comprises a protein-based polymer.

98. The biophotonic composition of claim 97, wherein the protein-based polymer is gelatin or collagen, or both.

99. The biophotonic composition of any one of claims 81 to 98, wherein the carrier medium comprises a polysaccharide.

100. The biophotonic composition of claim 99, wherein the polysaccharide is one or more of starch, chitosan, chitin, agar, an alginate, xanthan, carrageenan, guar gum, gellan gum, pectin, or locust bean gum.

101. The biophotonic composition of any one of claims 81 to 100, wherein the carrier medium comprises at least one glycol.

102. The biophotonic composition of claim 101, wherein the glycol is selected from the group consisting of ethylene glycol and propylene glycol.

103. The biophotonic composition of any one of claims 81 to 102, wherein the at least one fungal-derived chromophore is a fluorescent chromophore.

104. The biophotonic composition of claim 103, wherein the at least one fungal-derived chromophore absorbs and/or emits light within the visible range.

105. The biophotonic composition of claim 103, wherein the at least one fungal-derived chromophore absorbs and/or emits light within the green, orange and yellow portions of the electromagnetic spectrum.

106. The biophotonic composition of any one of claims 81 to 105, wherein the composition further comprises at least a second chromophore.

107. The biophotonic composition of claim 106, wherein the at least one fungal-derived chromophore has an emission spectrum that overlaps at least 20% with an absorption spectrum of the at least second chromophore.

108. The biophotonic composition of claim 106, wherein the at least one fungal-derived chromophore has an absorption spectrum that overlaps at least 20% with an emission spectrum of the at least second chromophore.

109. The biophotonic composition of claim 106, wherein the at least one fungal-derived chromophore transfers energy to the at least second chromophore upon illumination with a light.

110. The biophotonic composition of claim 106, wherein the at least second chromophore transfers energy to the at least one fungal-derived chromophore upon illumination with light.

111. The biophotonic composition of any one of claims 106 to 110, wherein the at least second chromophore is a xanthene dye.

112. The biophotonic composition of claim 111, wherein the xanthene dye is Eosin Y, Eosin B, Erythrosin B, Fluorescein, Rose Bengal, Phloxin B, or combinations thereof.

113. The biophotonic composition of claim 112, wherein the xanthene dye is Eosin Y.

114. The biophotonic composition of claim 112, wherein the xanthene dye is Rose Bengal.

115. The biophotonic composition of any one of claims 81 to 114, wherein the at least one fungal-derived chromophore is present in an amount of between about 0.0001% to about 40% by weight of the total composition, or between about 0.0001% to about 2% by weight of the total composition.

116. The biophotonic composition of any one of claims 81 to 115, wherein the biophotonic composition has a translucency of at least about 40%, about 50%, about 60%, about 70%, about 80%, about 90% or about 100% in a visible range without the chromophore(s).

117. The biophotonic composition of any one of claims 81 to 116, wherein upon exposure to light, the composition emits at least 1.25×, 1.5×, 1.75× or 2× more red, yellow and/or orange light than a composition lacking the at least one fungal-derived chromophore.

118. An article of manufacture comprising:
a) a photoactivatable fiber having a plurality of a photoactivatable strand that comprises a first thermoplastic polymer and a second thermoplastic polymer, and at least one photoactivatable agent;
wherein the first polymer forms a core along the length of the strand, and the second polymer forms a sheath surrounding the core along the length of the strand; and
wherein the at least one photoactivable agent absorbs and emits light between about 400 nm and about 800 nm; and
b) a biophotonic composition of any one of claims 1-37 and 137-153.

119. The article of manufacture of claim 118, wherein the first polymer and the second polymer is a material selected from any one or more of acrylic, acrylonitrile butadiene styrene (ABS), polybenzimidazole (PBI), polycarbonate, polyether sulfone (PES), polyetherether ketone, (PEEK), polyetherimide (PE1), polyethylene (PE), polyphenylene oxide (PPO), polyphenylene sulfide (PPS), polypropylene (PP), polystyrene, polyvinyl chloride (PVC), teflon, polybutylene, polyethylene terephthalate (PET), polybutylene terephthalate (PBT), nylon, polylactic acid (PLA), polymethyl methacrylate polyester, polyurethane, rayon, and poly(methyl methacrylate) (PMMA).

120. The article of manufacture of claim 118 or 119, wherein the first polymer and the second polymer are the same material.

121. The article of manufacture of claim 118 or 119, wherein the first polymer and the second polymer are different materials.

122. The article of manufacture of any one of claims 118-121, wherein the first polymer that forms the core comprises at least one photoactivatable agent.

123. The article of manufacture of claim 122, wherein the photoactivable agent is selected from one or more of Eosin Y, Eosin B, Erythrosine, Fluorescein, or Rose Bengal.

124. The article of manufacture of claim 123, wherein the photoactivatable agent is present in an amount in the range of about 0.1% to about 25% by weight.

125. The article of manufacture of claim 118, wherein the second polymer and first polymer are in an amount in any one of the following second polymer/first polymer ratios by weight: 10:90, 25:75, 50:50, or 75:25.

126. A photoactivatable fiber comprising:
a plurality of a strand that comprises a first thermoplastic polymer and a second thermoplastic polymer, and at least one photoactivatable agent associated with the first polymer;
wherein the first polymer forms a core along the length of the strand, and the second polymer forms a sheath surrounding the core along the length of the strand;
wherein a ratio by weight of the second polymer to the first polymer is in the range of about 5:95 to about 90:10;
wherein the at least one photoactivable agent absorbs and emits light between about 400 nm and about 800 nm.

127. The photoactivatable fiber of claim 126, wherein the second polymer and the first polymer are in a ratio amount of about 25:75, about 50:50, or about 72:25 second polymer/first polymer by weight.

128. The photoactivatable fiber of claim 126, wherein the first polymer and the second polymer is a material selected from any one or more of acrylic, acrylonitrile butadiene styrene (ABS), polybenzimidazole (PBI), polycarbonate, polyether sulfone (PES), polyetherether ketone, (PEEK), polyetherimide (PE1), polyethylene (PE), polyphenylene oxide (PPO), polyphenylene sulfide (PPS), polypropylene (PP), polystyrene, polyvinyl chloride (PVC), teflon, polybutylene, polyethylene terephthalate (PET), polybutylene terephthalate (PBT), nylon, polylactic acid (PLA), polymethyl methacrylate polyester, polyurethane, rayon, and poly(methyl methacrylate) (PMMA).

129. The photoactivatable fiber of any one of claims 126-128, wherein the first polymer and the second polymer are the same material.

130. The photoactivatable fiber of any one of claims 126-128, wherein the first polymer and the second polymer are different materials.

131. The photoactivatable fiber of any one of claims 126-130, wherein the at least one photoactivable agent is selected from one or more of Eosin Y, Eosin B, Erythrosine, Fluorescein, or Rose Bengal.

132. The photoactivatable fiber of any one of claims 126-131, wherein about 10 to about 360 strands form the fiber.

133. The photoactivatable fiber of any one of claims 126-132, wherein about 19 strands form the fiber.

134. The photoactivatable fiber of any one of claims 126-133, wherein the photoactivatable agent is present in the first polymer in an amount in the range of about 0.1% to about 25% by weight.

135. The photoactivatable fiber of any one of claims 126-134, wherein the photoactivatable agent is present in the first polymer at about 1% by weight.

136. A photoactivatable fabric comprising a plurality of a photoactivatable fiber of any one of claims 126-135.

137. A biophotonic composition comprising:
a) a fungal extract comprising at least one fungal-derived chromophore; and
b) a carrier medium comprising glycerin and propylene glycol.

138. The biophotonic composition of claim 137, wherein the at least one fungal-derived chromophore is derived from a Ganoderma species.

139. The biophotonic composition of claim 138, wherein the Ganoderma species is Ganoderma lucidum.

140. The biophotonic composition of any one of claims 137-139, wherein the composition comprises 4 parts fungal extract to 1 part carrier medium.

141. The biophotonic composition of any one of claims 137-140, wherein the glycerin is present in the carrier medium in a range of about 5% to about 25% by w/w.

142. The biophotonic composition of any one of claims 137-141, wherein the glycerin is present in the carrier medium at about 11% by w/w.

143. The biophotonic composition of any one of claims 137-142, wherein the propylene glylcol is present in the carrier medium in a range of about 40% to about 60% by w/w.

144. The biophotonic composition of any one of claims 137-143, wherein the propylene glylcol is present in the carrier medium at about 56% by w/w.

145. The biophotonic composition of any one of claims 137-144, further comprising at least a second chromophore.

146. The biophotonic composition of claim 145, wherein the at least second chromophore is a xanthene dye.

147. The biophotonic composition of claim 146, wherein the xanthene dye is Eosin Y, Eosin B, Rose Bengal, or a combination thereof.

148. The biophotonic composition of any one of claims 137-147, wherein the carrier medium further comprises an oxidant.

149. The biophotonic composition of claim 148, wherein the oxidant is urea peroxide.

150. The biophotonic composition of claim 149, wherein the urea peroxide is present in the carrier medium in a range of about 10% to about 20% by w/w.

151. The biophotonic composition of claim 150, wherein the urea peroxide is present in the carrier medium at about 16% by w/w.

152. The biophotonic composition of any one of claim 137-151, further comprising a thickening agent.

153. The biophotonic composition of claim 152, wherein the thickening agent is an ethylene oxide (EO)-propylene oxide (PO) block copolymers.

154. A method of treating a rare disease that afflicts skin or soft tissues comprising administering a biophotonic composition of any one of claims 137-153.

155. The method of claim 154, wherein the rare disease is selected from Hailey-Hailey syndrome, epidermolysis bullosa, CHILD syndrome, dermatomyositis, hidradenitis suppurativa, acquired ichthyosis, hereditary ichthyosis, lichen myxedematosus, scleromyxedema, pemphigus, a porphyria disorders, Ehlers-Danlos syndrome, cutis hyperelastica, eosinophilic fasciitis, osteogenesis imperfect, scleroderma, and Winchester syndrome.

156. The use of claim 155, wherein the rare disease is Hailey-Hailey syndrome.

157. The use of claim 155, wherein the rare disease is epidermolysis bullosa.

158. The use of claim 155, wherein the rare disease is hidradenitis suppurativa.

159. The use of claim 155, wherein the rare disease is scleroderma.
